(19) United States
(12) Patent Application Publication
(10) Pub. No.: US 2025/0222138 A1
(43) Pub. Date: Jul. 10, 2025

(54) SITE-SPECIFIC IN VIVO T CELL ENGINEERING, SYSTEMS, COMPOSITIONS AND METHODS THEREOF

(71) Applicant: RAMOT AT TEL-AVIV UNIVERSITY LTD., Tel Aviv (IL)

(72) Inventors: Adi BARZEL, Tel Aviv (IL); Inbal REUVENI, Tel Aviv (IL); Miriam FRIED, Tel Aviv (IL); Alessio D. NAHMAD, Tel Aviv (IL)

(21) Appl. No.: 18/852,404

(22) PCT Filed: Mar. 28, 2023
(86) PCT No.: PCT/IL2023/050323

§ 371 Date: Sep. 27, 2024

Related U.S. Application Data
(60) Provisional application No. 63/324,284, filed on Mar. 28, 2022.

(51) Int. Cl. A61K 48/00; C07K 14/725; C12N 9/22; C12N 15/11

(52) U.S. Cl. CPC A61K 48/0058; C07K 14/7051; C12N 9/22; C12N 15/11; C12N 2310/20

(57) ABSTRACT

The present disclosure relates to immunotherapy. In more specific embodiments, the present disclosure provides systems, compositions, methods and uses of viral vectors comprising nucleic acid sequence of interest that encodes at least one therapeutic product, and a nucleic acid sequence encoding at least one nuclease, for in vivo targeted insertion of the nucleic acid sequence of interest into a target locus within at least one cell of the T lineage.

### TECHNOLOGICAL FIELD

The invention relates to immunotherapy. More specifically, the invention relates to site specific in vivo engineering of cells of the T lineage using a site-specific nuclease, targeting the integration of exogenous nucleic acid sequence of interest into a desired target locus, systems compositions, methods and uses thereof in immunotherapy.

### BACKGROUND ART

References considered to be relevant as background to the presently disclosed subject matter are listed below:
[1] WO 2017/062451;
[2] US 2017/0298419 A1;
[3] Daniel T. MacLeod. Molecular Therapy Vol. 25 No 4; pages 949-961 (2017).

Acknowledgement of the above references herein is not to be inferred as meaning that these are in any way relevant to the patentability of the presently disclosed subject matter.

### BACKGROUND

Cancer immunotherapy harnesses and augments immune mechanisms to fight malignancies. In particular, adoptive T cell transfer entails the activation and expansion of T cells that target tumor associated antigens (TAA). Naturally occurring T cell receptors (TCRs) often have low affinity against TAAs. However, T cells can be engineered to express highly potent TCRs, which can recognize fragments of both intracellular and cell surface proteins when presented in a major histocompatibility complex (MHC) context. Alternatively, T cells can be engineered to express chimeric antigen receptors (CARs), which have a high TAA affinity. CARs recognize intact cell surface proteins in an MHC independent manner and are thus insensitive to cancer escape mechanisms associated with MHC loss. Second and third generation CARs have an increased potency due to inclusion of multiple co-stimulatory domains, allowing highly promising clinical results for several hematological malignancies. Treatment of solid tumors may in turn be facilitated by identifying and targeting cancer specific antigens, by using short-lived CAR mRNA to reduce toxicity and by counteracting inhibitory immune checkpoints. However, large scale application of adoptive T cell therapy may be extremely challenging. State of the art T cell engineering for cancer immunotherapy relies on ex vivo manipulations. It is conducted in a limited number of specialized medical centers, because it requires dedicated facilities and expertise in clinical grade collection, purification and activation of T cell products, as well as in viral vector manipulations. Autologous T cell engineering and expansion is time-consuming, delaying treatment of patients in need. Banking of engineered allogenic cells may reduce timelines and costs. Indeed, targeting exogenous nucleic acid sequences encoding CARs or TCRs into the TCR locus using homing nucleases were shown to reduce the variegated expression, random integration and premature T cell exhaustion associated with the commonly used integration techniques for CAR genes [1, 2, 3]. However, pre-conditioning chemotherapy may still be needed, and complex manipulations may be required to prevent graft-versus-host disease and to delay graft rejection.

There is therefore a crucial unmet need to design personalized and scalable immunotherapies to treat the very large population of patients with cancer and immune-related diseases.

The present invention addresses these needs by providing site-specific in vivo engineering of T cells within the patient's body.

### SUMMARY OF THE INVENTION

A first aspect of the present disclosure relates to a system or kit for in vivo targeted insertion of at least one exogenous nucleic acid sequence of interest into a target genomic locus of a cell of the T lineage in a mammalian subject. More specifically, the system or kit disclosed herein, may comprise the following component:

As component (a), at least one nucleic acid molecule comprising the at least one exogenous nucleic acid sequence of interest, and/or at least one cassette/s and/or construct/s thereof. The sequence is flanked on at least one of the 5′ and 3′ thereof by at least one homology arm, for integration to the target locus by homologous recombination. It should be noted that the at least one nucleic acid molecule and/or at least one cassette/s and/or construct/s is comprised within at least one AAV vector and/or AAV-like vector. The disclosed system further comprises as component (b), at least one site specific nuclease, or at least one nucleic acid sequence encoding the nuclease or any cassette/s and/or construct/s, and/or vector/s and/or delivery vehicle/s comprising the at least one nuclease or the nucleic acid sequence encoding said nuclease.

A further aspect of the present disclosure relates to a viral vector for in vivo targeted insertion of at least one exogenous nucleic acid sequence of interest into a target genomic locus of a cell of the T lineage in a mammalian subject. More specifically, the viral vector may comprise at least one of: (a), at least one nucleic acid molecule comprising at least one exogenous nucleic acid sequence of interest. The sequence is flanked on at least one of the 5′ and 3′ thereof by at least one homology arm, for integration to the target locus by homologous recombination; and/or (b), at least one site specific nuclease, or at least one nucleic acid sequence encoding the nuclease.

A further aspect of the present disclosure relates to a method for in vivo targeted insertion of at least one exogenous nucleic acid sequence of interest into a target genomic locus of a cell of the T lineage, in a mammalian subject. The disclosed method comprises the step of administering to the subject an effective amount of:

(a) at least one nucleic acid molecule comprising the at least one exogenous nucleic acid sequence of interest, and/or at least one cassette/s and/or construct/s thereof. The sequence is flanked on at least one of the 5′ and 3′ thereof by at least one homology arm, for integration to the target locus by homologous recombination. It should be noted that the at least one nucleic acid molecule and/or at least one cassette/s and/or construct/s is comprised within at least one AAV vector and/or AAV-like vector.

The subject is further administered with (b), at least one site specific nuclease, or at least one nucleic acid sequence encoding said nuclease or any cassette/s and/or construct/s, and/or vector/s and/or delivery vehicle/s comprising the at least one nuclease or said nucleic acid sequence encoding the nuclease.

Alternatively (c), the subject is administered with any system, delivery vehicle, matrix, nano- or micro-particle and/or composition comprising (a) and/or (b).

A further aspect of the present disclosure relates to a method for treating, preventing, ameliorating, inhibiting or delaying the onset of an immune-related disorder in a mammalian subject. Specifically, the method comprises the step of administering to said subject a therapeutically effective amount of:

(a), at least one nucleic acid molecule comprising at least one exogenous nucleic acid sequence of interest, and/or at least one cassette/s and/or construct/s thereof. The sequence is flanked on at least one of the 5′ and 3′ thereof by at least one homology arm, for integration by homologous recombination to at least one target locus of a cell of the T lineage in said subject. It should be noted that the at least one nucleic acid molecule and/or at least one cassette/s and/or construct/s is comprised within at least one AAV vector and/or AAV-like vector; and (b), at least one site specific nuclease, or at least one nucleic acid sequence encoding said nuclease or any cassette/s and/or construct/s, and/or vector/s and/or delivery vehicle/s comprising said at least one nuclease or said nucleic acid sequence encoding said nuclease; or (c), any system, vehicle, matrix, nano- or micro-particle and/or composition comprising (a) and/or (b).

A further aspect of the present disclosure provides nucleic acid cassette or vector for in vivo targeted insertion of at least one exogenous nucleic acid sequence of interest into at least one of the TRAC, and the TRBC loci of a cell of the T lineage in a mammalian subject. The cassette or vector disclosed herein comprise: (a) at least one nucleic acid molecule comprising the at least one exogenous nucleic acid sequence of interest, the sequence is flanked on at least one of the 5′ and 3′ thereof by at least one homology arm, for integration to said target site by homologous recombination; and (b) at least one nucleic acid sequence encoding at least one site specific nuclease.

A further aspect provided by the present disclosure relates to a therapeutically effective amount of: (a) at least one nucleic acid molecule comprising at least one exogenous nucleic acid sequence of interest, and/or at least one cassette/s and/or construct/s thereof. The sequence is flanked on at least one of the 5′ and 3′ thereof by at least one homology arm, for integration by homologous recombination to at least one target locus of a cell of the T lineage in said subject. It should be noted that the at least one nucleic acid molecule and/or at least one cassette/s and/or construct/s is comprised within at least one AAV vector and/or AAV-like vector; and (b), at least one site specific nuclease, or at least one nucleic acid sequence encoding the nuclease or any cassette/s and/or construct/s, and/or vector/s and/or delivery vehicle/s comprising said at least one nuclease or the nucleic acid sequence encoding said nuclease; or (c), any system, delivery vehicle, matrix, nano- or micro-particle and/or composition comprising (a) and/or (b); for use in a method for treating, preventing, ameliorating, inhibiting or delaying the onset of an immune-related disorder in a mammalian subject.

In a further aspect thereof, the present disclosure provides an effective amount of:

(a) at least one nucleic acid molecule comprising at least one exogenous nucleic acid sequence of interest, and/or at least one cassette/s and/or construct/s thereof. The sequence is flanked on at least one of the 5′ and 3′ thereof by at least one homology arm, for integration to said target locus by homologous recombination. It should be noted that the at least one nucleic acid molecule and/or at least one cassette/s and/or construct/s is comprised within at least one AAV vector and/or AAV-like vector; and (b) at least one site specific nuclease, or at least one nucleic acid sequence encoding said nuclease or any cassette/s and/or construct/s, and/or vector/s and/or delivery vehicle/s comprising said at least one nuclease or said nucleic acid sequence encoding said nuclease; or (c) any system, vehicle, matrix, nano- or micro-particle and/or composition comprising (a) and/or (b); for use in a method for in vivo targeted insertion of the at least one exogenous nucleic acid sequence of interest into a target genomic locus of a cell of the T lineage, in a mammalian subject.

A further aspect of the present disclosure relates to a genetically engineered cell of the T cell lineage, any population of cells comprising at least one the genetically modified cell, or any composition comprising said cell or population of cells. The cells may comprise a modified TRAC and/or TRBC loci comprising at least one exogenous nucleic acid sequence of interest. It should be noted that the cell was genetically modified by at least one nucleic acid cassette/s and/or construct/s, and/or vector/s and/or delivery vehicle/s comprising: (a) at least one nucleic acid molecule comprising said at least one exogenous nucleic acid sequence of interest, and/or at least one cassette/s and/or construct/s thereof. The sequence is flanked on at least one of the 5′ and 3′ thereof by at least one homology arm, for integration to the TRAC and/or TRBC loci by homologous recombination. It should be noted that the at least one nucleic acid molecule and/or at least one cassette/s and/or construct/s is comprised within at least one AAV vector and/or AAV-like vector; and (b) at least one nucleic acid sequence encoding at least one site specific nuclease.

A further aspect of the present disclosure relates to a composition comprising an effective amount of at least one system or nucleic acid cassette or vector, or any matrix, nano- or micro-particle thereof, for in vivo targeted insertion of at least one exogenous nucleic acid sequence of interest into a target genomic locus of a cell of the T lineage in a mammalian subject. The system and/or nucleic acid vector comprise: (a) at least one nucleic acid molecule comprising said at least one exogenous nucleic acid sequence of interest, and/or at least one cassette/s and/or construct/s thereof. The sequence is flanked on at least one of the 5′ and 3′ thereof by at least one homology arm, for integration to said target locus by homologous recombination. It should be noted that the at least one nucleic acid molecule and/or at least one cassette/s and/or construct/s is comprised within at least one AAV vector and/or AAV-like vector; and (b) at least one site specific nuclease or at least one nucleic acid sequence encoding the site-specific nuclease. The composition optionally further comprises at least one pharmaceutically acceptable carrier/s, excipient/s, auxiliaries, and/or diluent/s.

These and other aspects of the present invention will become apparent by the hands of the following disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to better understand the subject matter that is disclosed herein and to exemplify how it may be carried out in practice, embodiments will now be described, by way of non-limiting example only, with reference to the accompanying drawings, in which:

FIG. 1A-1C. Insertion of a human CD19 CAR into the TRAC locus using an AAV coding for the ARCUS nuclease

FIG. 1A(i). A scheme of the AAV construct. The ARCUS nuclease is episomally expressed, leading to a DSB at the TRAC locus. A CAR cassette flanked by homology arms (HAs), is integrated at the site and expressed under the TRAC endogenous promoter. For example: HA-SA-Furin-2A-CAR-PolyA-HA-JET ARCUS-PolyA.

FIG. 1A(ii). A scheme of an alternative strategy is an episomal and constitutive CAR expression, proceeded by 2A peptide and SD, (when all flanked by homology arms) that leads to splicing and episomal expression of ARCUS nuclease and genomic CAR integration.

For example: HA-PolyA-JET CAR-Furin-2A-SD-HA-SA-ARCUS-polyA.

FIG. 1A(iii). A scheme of another optional strategy is an episomal expression of ARCUS with a SD that leads to splicing and both episomal and genomic expression of CAR under TRAC promoter.

For example: JET ARCUS-Furin-SD-HA-SA-2A-CAR-PolyA-HA.

FIG. 1B. The anti-CD8 DARPIN was genetically fused into the GH2-GH3 surface loop of the VP1 capsid gene of AAV-DJ. In order to disrupt heparin sulfate binding, Arginines R587 and R590 were further mutated to Alanine residues in the plasmid encoding the VP1-DARPIN fusion. The splice acceptor (SA) is inactivated to prevent incorporation of DARPIN into the VP2 and VP3 capsid proteins. Expression of unmodified VP2 and VP3 is provided by a second plasmid, in which the start codon (Met) of VP1 was inactivated to prevent incorporation of unmodified VP1 in the capsid.

FIG. 1C. Western Blot of AAV_CD8_DARPIN preps demonstrates the expected size of the edited VP1-DARPIN compared to the w.t. VP1 of unmodified AAV.

FIG. 2A-2B. Specificity of human cell transduction by a GFP coding AAV DJ vector, with or without CD8_DARPIN incorporation FIG. 2A. 293T cells were transduced with a GFP coding AAV-DJ vector with disrupted heparin sulfate binding and with an incorporated CD8_DARPIN at an MOI of 56K AAV genomes per cell. Fluorescence was measured 48 h later by flow cytometry.

FIG. 2B(i)-(ii). Human primary T cells were transduced with a GFP coding AAV-DJ with an untargeted AAV (FIG. 2B(i)) or incorporated CD8_DARPIN (FIG. 2B(ii)), at an MOI of 56K AAV genomes per cell. The panel shows CD4+ and CD8+ cells gated out of (Singlets+GFP+). Florescence was measured 48 h later by flow cytometry.

FIG. 3A-3G. Expression and activity of a CAR anti-human CD19, delivered and targeted using an ARCUS-CAR coding AAV

FIG. 3A. Genomic integration of the CAR into the TRAC locus.

FIG. 3B. Sanger sequencing of PCR product of ARCUS-CAR targeted transduction. The locus sequence is shown by the double strand sequence as shown by SEQ ID NO: 43 and the complementary SEQ ID NO: 44. Sanger sequencing of the nucleic acid sequence of SEQ ID NO: 43, is denoted by SEQ ID NO: 45.

FIG. 3C-3E. CAR expression following transduction of an ARCUS-CAR coding AAV (targeted to CD8+ or untargeted, gated out of live, singlets, CD8+ or CD4+). Cells were expanded in co-culture with CD19 expressing NALM6 cells (CD19+) (FIG. 3E) or expansion with U937 (CD19−) cells (FIG. 3D) or left with no expansion (FIG. 3C).

FIG. 3F. shows CAR expression and TCR knockout, quantification of FIGS. 3C, 3D and 3E.

FIG. 3G. IFN-gamma secretion following human T cell transduction with an ARCUS-CAR coding AAV (targeted to CD8+ or untargeted). Transduced cells were co-incubated with U937 (CD19−) or with NALM6 (CD19+), at an MOI of 500K vector genomes per cell.

FIG. 4A-4E. Site specific in vivo T cell engineering to express CAR

FIG. 4A. Site specific in vivo T cell engineering to express CAR. General scheme of strategy number 1.

FIG. 4B. General scheme of the in vivo experiment. T cell engineering.

FIG. 4C. Follow up of NALM6-LUC cells by IVIS to monitor tumor volume in mice.

FIG. 4D(i)-(iv). Specific expansion of CD8 T cells with reduced CD3 expression due to TRAC disruption. AAV and control mice were sacrificed, and cells were extracted from the BM. Cells were stained for CAR and CD3.

FIG. 4E(i)-(iii). Genomic integration of the CAR into the TRAC locus in AAV treated mouse (FIG. 4E(i), (ii) upper panels. Sanger sequencing of the PCR product of ARCUS-CAR targeted transduction of the liver in AAV mouse (FIG. 4E(i),(ii) lower panels). In FIG. 4E(i), the locus sequence is shown by the double strand sequence as shown by SEQ ID NO: 46 and the complementary SEQ ID NO: 47. Sanger sequencing of the nucleic acid sequence of SEQ ID NO: 46, is denoted by SEQ ID NO: 48. In FIGS. 4E(ii), and 4E(iii), the locus sequence is shown by the double strand sequence as shown by SEQ ID NO: 49 and the complementary SEQ ID NO: 50. Sanger sequencing of the nucleic acid sequence of SEQ ID NO: 49, is denoted by SEQ ID NO: 52. The predicted amino acid sequence is denoted by SEQ ID NO: 51.

### DETAILED DESCRIPTION OF EMBODIMENTS

Current T cell engineering for immunotherapy has shown great clinical success, but it cannot be applied to most patients in need, because it relies on cumbersome and expensive ex vivo manipulations, which are performed in specialized centers only. The use of engineered allogenic cells may reduce timelines and costs. However, pre-conditioning chemotherapy may still be needed, and complex manipulations may be required to prevent graft-versus-host disease and to delay graft rejection.

More specifically, the enormous treatment costs have become the main challenge in gene therapy currently preventing that the whole potential of this revolutionary treatment become available for European and global health. Becoming accessible and affordable, not only for a few people, but for most patients in need, is the next important step gene therapy must take. Main driver of the high cost is the patient individualized character of the ex vivo gene therapy approach in which autologous patient cells must be genetically modified ex vivo in cell culture, as it is e.g. the case for CAR T cell therapy, which revolutionized treatment options for end stage lymphoma patients. This will radically change when therapeutic genes can be delivered precisely to the therapy-relevant cells upon direct administration into the patient's body. Then, the currently highly individualized medicinal product will convert into a universally applicable drug. Similar targeting schemes ex vivo were previously shown to reduce the variegated expression, random integration and premature T cell exhaustion associated with the commonly used integration techniques for CAR genes (retro vectors, lentivectors or transposons).

The present disclosure provides methods for the site-specific in vivo engineering of T cells within the patient's body using systemic vector injections. This approach revolutionizes immunotherapy by providing safe potent and scalable methods for site specific in vivo T cell engineering. Disclosed are methods for the site-specific in vivo engineering of T cells, obviating pre-conditioning and reducing timelines and expenses without risking GVHD or graft rejection. The present design uniquely allows the synergy between the benefits of in vivo T cell engineering and the benefits of site-specific T cell engineering.

In particular, vectors coding for a desired exogenous product, e.g., any therapeutic product, for example, any receptor molecule such as a CAR molecule or an exogenous and/or engineered TCR molecule. In addition, a site-specific nuclease, targeting the integration of the desired nucleic acid sequence/s of interest encoding the product of interest (e.g., CAR and/or TCR) into a desired locus is provided to a subject (e.g., by injection). The site-specific endonuclease is either coded on the same vector and/or cassette as the desired at least one exogenous product or is provided in a separate co-administered (co-injected) vector. In some embodiments, a single vector, e.g., the adeno associated viral vector (AAV) is used to code for both at least one desired product, e.g., a therapeutic product, for example, a receptor molecule such as CAR molecule, and a nuclease targeting the desired product (e.g., CAR) into a desired locus. In some embodiment, the nuclease is a homing endonuclease, or an engineered derivative thereof. In yet some further embodiments, the desired locus is the TCR alpha constant (TRAC) locus. In some embodiments, the vectors are further engineered to bear targeting moieties, for example, moieties promoting the preferred transduction of T cells in vivo.

Thus, in a first aspect, the present disclosure relates to a system and/or kit for in vivo targeted insertion of at least one exogenous nucleic acid sequence of interest into a target genomic locus of a cell of the T lineage in a mammalian subject. More specifically, the system or kit disclosed herein, may comprise the following components: As component (a), at least one nucleic acid molecule comprising the at least one exogenous nucleic acid sequence of interest, and/or at least one cassette/s and/or construct/s thereof. The sequence is flanked on at least one of the 5′ and/or 3′ thereof by at least one homology arm, for integration into the target locus, for example, by homologous recombination. It should be noted that the at least one nucleic acid molecule and/or at least one cassette/s and/or construct/s is comprised within at least one AAV vector and/or AAV-like vector.

The disclosed systems and/or kits further comprise as component (b), at least one site specific nuclease, or at least one nucleic acid sequence encoding the nuclease or at least one cassette, and/or construct, and/or vector and/or delivery vehicle comprising the at least one nuclease or the nucleic acid sequence encoding the nuclease.

As indicated herein, the present disclosure provides systems and/or kits adapted for the insertion of the nucleic acid sequence of interest into a specific locus. More particularly, as opposed to a random insertion or incorporation of nucleic acid molecule into a genomic sequence at any random site, the targeted insertion disclosed by the present disclosure, involves the particular identification and insertion of the exogeneous nucleic acid sequence into a predetermined and specific targeted site. The recognition and specificity determination of the targeted insertion is enabled at least in part, in some embodiments by the use of at least one homology arm that flanks the nucleic acid sequence of interest to be inserted. Homology arm/s as used herein, refer to the sequence in the nucleic acid molecule provided by the disclosed system, that is similar or identical to the sequences found in a target region of interest. Homology arms are typically used in gene targeting or gene editing techniques, to introduce specific genetic changes into the target region. The homology arms serve as templates for recombination, allowing for precise insertion or deletion of genetic material. Designing homology arms that are complementary to the sequences flanking the target region, facilitates and enables accurate and efficient integration of the desired nucleic acid sequence into the target site.

As indicated above, in some embodiments, the present disclosure concerns systems and methods for targeted insertion of at least one nucleic acid molecule that comprise at least one exogenous nucleic acid sequence of interest to a specific target locus, the nucleic acid sequence of interest may be inserted and/or integrated in the target locus and/or target site by homologous recombination (HDR). HDR occurs between two DNA molecules that display regions of significant similarity. During homologous recombination, a DNA double strand break is created in one of the DNA molecules, and the resulting free ends of the broken strand invade the complementary region of the other DNA molecule. This forms a “crossing-over” point where the two molecules are joined together.

In some embodiments, specifically when the nucleic acid sequence of interest provided by the nucleic acid molecule of the disclosed systems and/or kits, is incorporated into the target nucleic acid sequence via HDR, the donor nucleic acid molecule may also comprise, or in some embodiments, may be specifically flanked by, at least one homology arm, that display complementarity to a nucleic acid sequence flanking the target site for incorporation. In some embodiments, the homology arms flank any exogenous nucleic acid sequence of interest that is to be incorporated and integrated in the specific target site.

Still further, the targeted insertion of the nucleic acid sequence of interest provided by the nucleic acid molecule of the disclosed system/s and/or kit/s into a target locus in a target cell is performed “in vivo”. The term “in vivo”, within the living, refers to biological processes, herein, the incorporation of a nucleic acid sequence into a target locus, that takes place within a living organism or a natural environment. In some embodiments, the in vivo targeted insertion of the sequence of interest may be performed in a living body of any mammalian subject.

It should be noted that “target genomic locus” is the site of modification of an endogenous chromosomal locus by the insertion into, integration into, deletion of, or replacement of the endogenous target sequence, indicated herein as a “target locus”, via the in vivo targeted insertion using the system/kit, cassette, compositions and methods of the present disclosure. As used herein, a “target locus” is a region of DNA into which a nucleic acid sequence of interest is integrated, inserted and recombined within e.g. a region of DNA in a target cell. In some specific embodiments the target locus may be within the chromosomal DNA of the target cell. Thus, in some embodiments, the target locus is a genomic locus. A genomic locus, as used herein, refers to a specific physical location or position on a chromosome that is associated with a particular gene or DNA sequence. It is typically identified by its position on a linear map of the chromosome, which is determined by measuring the distance between two recognizable landmarks (typically, two other genes or genetic markers with known positions).

As used herein, a “target gene” or “endogenous gene” or “gene at a target locus” is a gene that naturally exists at a locus of integration, i.e. the gene that is endogenous to the target locus.

The target site in some embodiments may be located at any region of the target gene in any target locus. In some embodiments, the target site for targeted integration may be located within any coding and/or non-coding sequence of the target gene. In yet some further embodiments, the target site may be located in any coding sequence of the target gene of the target locus. Still further, in some embodiments, the target site for integration of the nucleic acid molecule of interest provided by the present disclosure, may be located at any non-coding sequence of the target gene in the target locus. Still further, in some embodiments, the target site for integration of the nucleic acid molecule of interest provided by the present disclosure, may be located at any coding sequence of the target gene in the target locus. In some embodiments, the target site for integration of the nucleic acid molecule of interest provided by the present disclosure, may be located at any exon of the target gene in the target locus. Still further, in some embodiments, the target site for integration of the nucleic acid molecule of interest provided by the present disclosure, may be located at any intron of the target gene in the target locus.

In some embodiments, the target locus targeted by the disclosed system may be at least one of: T cell receptor (TCR) α chain locus, TCRβ chain locus, TCRγ chain locus and the TCRS chain locus.

T cell receptors are composed of an alpha chain and a beta chain. More specifically, TCRβ chain locus, TCRα chain locus, TCRγ chain locus and the TCRδ chain locus refers to the chromosomal position of the TCRβ chain coding gene (on chromosome 7), of the TCRα chain coding gene (on chromosome 14), of the TCRγ chain coding gene (on chromosome 7) and of the TCRδ chain coding gene (on chromosome 14), respectively. The κ segments are on human chromosome 2 and the λ segments are on human chromosome 22.

In more specific embodiments, the target locus targeted by the disclosed system is at least one of the TCR alpha constant (TRAC), and the TCR beta constant (TRBC) loci.

The TCR (T-cell receptor) constant locus refers to the region of DNA that encodes the constant regions of the alpha (Cu) and beta (CO) chains of the TCR protein. The constant regions of the TCR chains are important for the structure and function of the TCR and play a role in signaling and activation of T cells. The constant regions of the TCR chains are encoded by separate genes, and each gene is located on a different chromosome. The Cu gene (TRAC) is located on chromosome 14, and the CO gene (TRBC) is located on chromosome 7.

Still further, in some embodiments, the TRAC gene may be the human TRAC gene, located at chromosome 14 position 22547506-22552156, and may comprise the nucleic acid sequence as denoted by Genebank accession number ENST00000611116.2.

In yet some further embodiments, the TRBC gene may be the human TRBC1 gene located at chromosome 7 position 142,791,694-142,793,368 and may comprise the nucleic acid sequence as denoted by may comprise the nucleic acid sequence as denoted by denoted Genebank accession number ENST00000633705.1.

In yet some further embodiments, the TRBC gene may be the human TRBC2 gene located at chromosome 7 position 142,498,725-142,500,432 and may comprise the nucleic acid sequence as denoted by may comprise the nucleic acid sequence as denoted by denoted Genebank accession number ENST00000466254.1.

In yet some further embodiments, the target site for the targeted insertion of the nucleic acid sequence of interest may be any site at any part of the TRAC gene. In some embodiments, the target site for the insertion of the nucleic acid sequence of interest may be within any one of the exons of the TRAC. Still further, in some embodiments, the TRAC gene consists of a total of four exons, which are numbered 1 to 4. Exons 1 and 2 encode the leader peptide and the variable region of the protein, respectively, while exons 3 and 4 encode the constant region of the protein. Thus, the target site may be within any sequence of exon 1, exon 2, exon 3 or exon 4 of the TRAC gene. In some specific embodiments, the target site may be located within exon 1 of the TRAC. In yet some further embodiments, the target site may be located at TRAC exon 1, that may comprise the nucleic acid sequence as denoted by SEQ ID NO: 39, or any homologs and variants thereof. In some alternative embodiments, the target site for the insertion of the nucleic acid sequence of interest may be within any one of the introns of the TRAC. Still further, in some embodiments, the target site for the insertion of the nucleic acid sequence of interest may be within any one of the regulatory sequences of the TRAC gene.

In yet some further embodiments, the target locus may be within the TRBC locus. Thus, in some embodiments, the target site for the targeted insertion of the nucleic acid sequence of interest may be any site at any part of the TRBC1 gene, or the TRBC2 gene. In some embodiments, the target site for the insertion of the nucleic acid sequence of interest may be within any one of the exons of the TRBC1 gene, or the TRBC2 gene. The TRBC1 gene, or the TRBC2 gene consists of a total of four exons, which are numbered 1-4. Exons 1 and 2 encode the leader peptide and the variable region of the protein, respectively, while exons 3 and 4 encode the constant region of the protein. Thus, the target site may be within any sequence of exon 1, exon 2, exon 3 or exon 4 of the TRBC1 gene, or the TRBC2 gene. In some alternative embodiments, the target site for the insertion of the nucleic acid sequence of interest may be within any one of the introns of the TRBC1 gene, or the TRBC2 gene. Still further, in some embodiments, the target site for the insertion of the nucleic acid sequence of interest may be within any one of the regulatory sequences of the TRBC1 gene, or the TRBC2 gene.

In some embodiments, the target locus targeted by the system of the present disclosure is the TRAC locus. In such case, the at least one homology arm flanking the nucleic acid sequence of interest enables the integration of the at least one exogenous nucleic acid sequence of interest into the TRAC locus.

In some specific and non-limiting embodiments, homolog arms applicable in the system of the present disclosure may comprise at least one left homology arm, and/or right homology arm. As indicated above, Homology arms, are DNA sequences that flank a nucleic acid sequence of interest and share significant sequence similarity or identity with the target genomic region to be modified. Still further, in some embodiments, homology arms may be designed to be complementary to the sequences in the target site, specifically, sequences that flank the genomic target site in the target locus, allowing for precise integration of the desired DNA fragment into the genome. The length and specificity of the homology arms are critical factors in the efficiency and accuracy of genome editing. Generally, longer homology arms improve the efficiency of homologous recombination and gene targeting, as they increase the likelihood of homologous pairing and reduce the frequency of random integration events.

In some embodiments, the nucleic acid molecule of interest (also referred to herein as a donor nucleic acid molecule), may be flanked by at least one homology arm. The term “flanked” as used herein refers to a nucleic acid sequence positioned between two defined regions. For example, as indicated above, the nucleic acid molecule of interest is flanked by a first and/or a second, for example, “left” and “right” homology arms, located at the 5′ end and the 3′ end, or upstream and downstream, respectively, of the nucleic acid molecule of interest. Homology arms comprise sequences that display homology to the target site. In some embodiments, the homology arm may display complementarity, at least partial complementarity, to sequences that flank the target site within a target nucleic acid sequence. It should be understood that in some embodiments, the homology arms may flank any exogenous sequence that should be incorporated in the target genomic site. Still further, the exogeneous sequence of interest may further comprise additional control elements that may facilitate transcription and/or translation and/or processing thereof (e.g., cleavage, tags). Thus, in some embodiments, the homology arms may flank the sequence that includes the nucleic acid sequence of interest and the additional control sequences.

In yet some further specific embodiments, the left homology arm may comprise the nucleic acid sequence as denoted by SEQ ID NO: 9, or any homologs or variants thereof.

In yet some further embodiments, the right homology arm used in the systems of the present disclosure may comprise the nucleic acid sequence as denoted by SEQ ID NO: 10, or any homologs or variants thereof.

The disclosed systems and/or kits, may use at least one nuclease for the in vivo targeted insertion of at least one nucleic acid sequence into a target site in a target genomic locus of cells of the T lineage in a mammalian subject. The nuclease may be provided either in encoding nucleic acid molecule that comprises nucleic acid sequence/s encoding at least one nuclease, or as a protein. In yet some further embodiments, the nuclease may be provided as a nucleic acid sequence either separately, or together with the nucleic acid sequence of interest that encodes in some embodiments, at least one therapeutic product, for example, a receptor molecule such as a CAR T molecule, or exogenous and/or engineered TCR molecule.

Nuclease as referred to herein, relates to an enzyme that in some embodiments display a nucleolytic activity, specifically, capable of cleaving the phosphodiester bonds between monomers of nucleic acids (e.g., DNA and/or RNA). Nucleases variously effect single and double stranded brakes in their target molecules. There are two primary classifications based on the locus of activity. Exonucleases digest nucleic acids from the ends. Endonucleases act on regions in the middle of target molecules. They are further subcategorized as deoxyribonucleases and ribonucleases. The former acts on DNA, the latter on RNA. The nucleases belong just like phosphodiesterase, lipase and phosphatase to the esterases, a subgroup of the hydrolases. This subgroup includes the Exonucleases which are enzymes that work by cleaving nucleotides one at a time from the end (exo) of a polynucleotide chain. A hydrolyzing reaction that breaks phosphodiester bonds at either the 3′ or the 5′ end occurs. Eukaryotes and prokaryotes have three types of exonucleases involved in the normal turnover of mRNA: 5′ to 3′ exonuclease (Xrn1), which is a dependent decapping protein; 3′ to 5′ exonuclease, an independent protein; and poly (A)-specific 3′ to 5′ exonuclease. Members of this family include Exodeoxyribonucleases producing 5′-phosphomonoesters, Exoribonucleases producing 5′-phosphomonoesters, Exoribonucleases producing 3′-phosphomonoesters and Exonucleases active with either ribo- or deoxy-. Members of this family include exonuclease, II, III, IV, V, VI, VII, and VIII. As noted above, Endonucleases are enzymes that cleave the phosphodiester bond within a polynucleotide chain. Some endonucleases, such as deoxyribonuclease I, cut DNA relatively nonspecifically (without regard to sequence), while many, typically called restriction endonucleases or restriction enzymes, cleave only at very specific nucleotide sequences.

In yet some further embodiments, the site-specific nuclease comprised within the system of the present disclosure is at least one of: at least one homing endonuclease, at least one zinc-finger nucleases (ZFNs), at least one transcription activator-like effector nuclease (TALEN), at least one clustered regulatory interspaced short palindromic repeat (CRISPR)/CRISPR associated (Cas) protein, and at least one Meganuclease-transcription activator-like (Mega-TAL).

In some particular embodiments, the nuclease used herein is at least one programmable engineered nuclease (PEN). In some embodiments, the PEN may comprise CRISPR/Cas, ZFN, TALEN, etc.

Still further in some embodiments, the nucleases applicable in the present disclosure may comprise at least one site specific nuclease/s. Site-specific nucleases cleave nucleic acid sequences (DNA, RNA), at specific locations within the genome or transcriptome. These nucleases are able to recognize and bind to specific DNA or RNA sequences, and then introduce a double-stranded break in the DNA or RNA at or near the site of recognition. Site-specific nucleases include a variety of enzymes, such as zinc finger nucleases (ZFNs), transcription activator-like effector nucleases (TALENs), and the CRISPR-Cas system, discussed herein after. In some embodiments, meganucleases may be used as nucleases in the systems and methods of the present disclosure. Homing endonucleases represent one of four types of genome-editing technologies; the other three are ZFNs (zinc-finger nucleases), TALENs (transcription activator-like effector nucleases), and CRISPR/Cas9 (clustered regularly interspaced short palindromic repeats with the Cas9 endonuclease).

“Meganucleases” or “homing endonucleases” are a group of naturally occurring nucleases which recognize 15-40 base-pair cleavage sites commonly found in the genomes of plants and fungi, that is frequently associated with parasitic DNA elements, such as group I self-splicing introns and inteins. They naturally promote homologous recombination or gene insertion at specific locations in the host genome by producing a double-stranded break in the chromosome, which recruits the cellular DNA-repair machinery. Meganucleases are commonly grouped into four families: the LAGLIDADG (SEQ ID NO: 1) family, the GIY-YIG family, the His-Cys box family and the HNH family. These families are characterized by structural motifs, which affect catalytic activity and recognition sequence. For instance, members of the LAGLIDADG (SEQ ID NO: 1) family are characterized by having either one or two copies of the conserved LAGLIDADG (SEQ ID NO: 1) motif. The LAGLIDADG meganucleases with a single copy of the LAGLIDADG (SEQ ID NO: 24) motif form homodimers, whereas members with two copies of the LAGLIDADG (SEQ ID NO: 1) motif are found as monomers.

Still further, genetic modification of genomic DNA can be performed using site-specific, rare-cutting endonucleases that are engineered to recognize DNA sequences in the locus of interest. Methods for producing engineered, site-specific endonucleases are known in the art. For example, zinc-finger nucleases (ZFNs) can be engineered to recognize and cut predetermined sites in a genome. ZFNs are chimeric proteins comprising a zinc finger DNA-binding domain fused to the nuclease domain of the Fokl restriction enzyme. The zinc finger domain can be redesigned through rational or experimental means to produce a protein that binds to a pre-determined DNA sequence—18 base pairs in length. By fusing this engineered protein domain to the Fokl nuclease, it is possible to target DNA breaks with genome-level specificity. ZFNs have been used extensively to target gene addition, removal, and substitution in a wide range of eukaryotic organisms. In some embodiments, ZFNs may be used as nucleases in the systems and methods of the present disclosure. Likewise, TAL-effector nucleases (TALENs) can be generated to cleave specific sites in genomic DNA. Like a ZFN, a TALEN comprises an engineered, site-specific DNA-binding domain fused to the Fokl nuclease domain. In this case, however, the DNA binding domain comprises a tandem array of TAL-effector domains, each of which specifically recognizes a single DNA base pair. A limitation that ZFNs and TALENs have for the practice of the current invention is that they are heterodimeric, so that the production of a single functional nuclease in a cell requires co-expression of two protein monomers. In some embodiments, TALENs may be used as the nucleases in the systems and methods of the present disclosure.

Compact TALENs have an alternative endonuclease architecture that avoids the need for dimerization. A Compact TALEN comprises an engineered, site-specific TAL-effector DNA-binding domain fused to the nuclease domain from the I-TevI homing endonuclease. Unlike Fokl, I-TevI does not need to dimerize to produce a double-strand DNA break so a Compact TALEN is functional as a monomer.

Engineered endonucleases based on the CRISPR/Cas9 system are also known in the art, and are also applicable for the systems/kits, compositions and methods of the present disclosure. A CRISPR endonuclease comprises two components: (1) a cas effector nuclease, typically microbial Cas9; and (2) a short “guide RNA” comprising a −20-nucleotide targeting sequence that directs the nuclease to a location of interest in the genome. By expressing multiple guide RNAs in the same cell, each having a different targeting sequence, it is possible to target DNA breaks simultaneously to multiple sites in the genome. Thus, CRISPR/Cas9 nucleases are suitable for the present disclosure.

As specified above, in some embodiments, the nuclease used in the disclosed systems and/or kits, compositions, methods and uses for the in vivo targeted insertion of the nucleic acid sequence comprising the desired nucleic acid sequence of interest may comprise at least one component of the CRISPR-Cas system. In should be understood that in some embodiments, the nucleic acid sequence of interest may encode at least one therapeutic and/or modulatory molecule. In some embodiments, the nucleic acid sequence of interest, encodes at least one therapeutic protein. In more specific embodiments, the therapeutic protein may be a receptor molecule. Still further in some embodiments, such receptor molecule may be a CAR molecule and/or a TCR molecule as disclosed herein. Still further, the Clustered Regularly Interspaced Short Palindromic Repeats (CRISPR) system, as used herein, is a bacterial immune system that has been modified for genome engineering. CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI.

It should be understood that the invention contemplates the use of any of the known CRISPR systems, particularly and of the CRISPR systems disclosed herein. The CRISPR-Cas system has evolved in prokaryotes to protect against phage attack and undesired plasmid replication by targeting foreign DNA or RNA. In bacterial immunity, the CRISPR-Cas system, targets DNA molecules based on short homologous DNA sequences, called spacers that have previously been extracted by the bacterium from the foreign pathogen sequence and inserted between repeats as a memory system. These spacers are transcribed and processed and this RNA, named crRNA or guide-RNA (gRNA), guides CRISPR-associated (Cas) proteins to matching (and/or complementary) sequences within the foreign DNA, called proto-spacers, which are subsequently cleaved. The spacers, or other suitable constructs or RNAs can be rationally designed and produced to target any DNA sequence. Moreover, this recognition element may be designed separately to recognize and target any desired target including outside of a bacterium.

In some specific embodiment, the CRISPR-Cas proteins useful in the present disclosure may be of a CRISPR Class 2 system. In yet some further particular embodiments, such class 2 system may be any one of CRISPR type II, and type V systems. In certain embodiments, the Cas applicable in the present invention may be any Cas protein of the CRISPR type II system. The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein. It should be appreciated that any type II CRISPR-Cas systems may be applicable in the disclosed systems and/or kits, compositions, methods and/or uses of the present disclosure, specifically, any one of type II-A or B. Thus, in yet some further and alternative embodiments, at least one cas gene useful in the methods and systems of the invention may be at least one cas gene of type II CRISPR system (either typeII-A or typeII-B). In more particular embodiments, at least one cas gene of type II CRISPR system useful for the methods and systems of the present disclosure may be the cas9 gene.

According to such embodiments, the CRISPR-Cas proteins that may be applicable in the systems and/or kits, compositions, methods and uses of the present disclosure, may be a CRISPR-associated endonuclease 9 (Cas9). Double-stranded DNA (dsDNA) cleavage by Cas9 is a hallmark of “type II CRISPR-Cas” immune systems. The CRISPR-associated protein Cas9 is an RNA-guided DNA endonuclease that uses RNA:DNA complementarity to a target site (proto-spacer). After recognition between Cas9 and the target sequence double stranded DNA (dsDNA) cleavage occurs, creating the double strand breaks (DSBs).

CRISPR type II system as used herein requires the inclusion of two essential components: a “guide” RNA (gRNA) and a CRISPR-associated endonuclease (Cas9). The gRNA is an RNA molecule composed of a “scaffold” sequence necessary for Cas9-binding (also named tracrRNA) and about 20 nucleotide long “spacer” or “targeting” sequence, which defines the genomic target to be modified. Guide RNA (gRNA), as used herein refers to a synthetic fusion or alternatively, annealing of the endogenous tracrRNA with a targeting sequence (also named crRNA), providing both scaffolding/binding ability for Cas9 nuclease and targeting specificity. Also referred to as “single guide RNA” or “sgRNA”. In yet some further particular embodiments, the class 2 system in accordance with the invention, may be a CRISPR type V system. In a more specific embodiment, the RNA guided DNA binding protein nuclease may be CRISPR-associated endonuclease X (CasX) system or CRISPR-associated endonuclease 14 (Cas14) system or CRISPR-associated endonuclease F (CasF, also known as Cas12j) system. The type V CRISPR-Cas systems are distinguished by a single RNA-guided RuvC domain-containing nuclease. As with type II CRISPR-Cas systems, CRISPR type V system as used herein requires the inclusion of two essential components: a gRNA and a CRISPR-associated endonuclease (CasX/Cas14/CasF). The gRNA is a short synthetic RNA composed of a “scaffold” sequence necessary for CasX/Cas14/CasF-binding and about 20 nucleotide long “spacer” or “targeting” sequence, which defines the genomic target to be modified. It should be noted that the gRNA used herein may comprise between about 3 nucleotides to about 100 nucleotides, specifically, 3, 4, 5, 6, 7, 8, 9, 10, 15, 20, 25, 30, 35, 40, 45, 50, 55, 60, 65, 70, 75, 80, 85, 90, 95 100 or more. More specifically between about 10 nucleotides to 70 nucleotides or more.

It should be noted that any CRISPR/Cas proteins may be useful in the disclosed systems and/or kits, compositions, methods and uses of the present disclosure, in some embodiments of the present disclosure, the endonuclease may be a Cas9, CasX, Cas12, Cas13, Cas14, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM10152, Rhodococcus erythropolis PR4, Rhodococcus jostii RFIA1, Rhodococcus opacus B4 (uid36573), Acidothermus cellulolyticus 11 B, Arthrobacter chlorophenolicus A6, Kribbella flavida (DSM 17836), Thermomonospora curvata (DSM43183), Bifidobacterium dentium Bd1, Bifidobacterium longum DJO10A, Slackia heliotrinireducens (DSM 20476), Persephonella marina EX H 1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Pei191, uncultured Termite group 1 bacterium phylotype Rs D17, Fibrobacter succinogenes 585, Bacillus cereus (ATCC 10987), Listeria innocua, Lactobacillus casei, Lactobacillus rhamnosus GG, Lactobacillus salivarius UCC118, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS10565, Streptococcus gallolyticus UCN34 (uid46061), Streptococcus gordonii Challis subst CH1, Streptococcus mutans NN2025 (uid46353), Streptococcus mutans, Streptococcus pyogenes M1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS6180, Streptococcus pyogenes MGAS315, Streptococcus pyogenes SSI-1, Streptococcus pyogenes MGAS10750, Streptococcus pyogenes NZ131, Streptococcus thermophiles CNRZ1066, Streptococcus thermophiles LMD-9, Streptococcus thermophiles LMG 18311, Clostridium botulinum A3 Loch Maree, Clostridium botulinum B Eklund 17B, Clostridium botulinum Ba4 657, Clostridium botulinum F Langeland, Clostridium cellulolyticum H10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus, moniliformis (DSM 12112), Bradyrhizobium BTAil, Nitrobacter hamburgensis X14, Rhodopseudomonas palustris BisB18, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 JGI, Azospirillum B510 (uid46085), Rhodospirillum rubrum (ATCC 11170), Diaphorobacter TPSY (uid29975), Verminephrobacter eiseniae EFO1-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 81116, Campylobacter jejuni, Campylobacter lari RM2100, Helicobacter hepaticus, Wolinella succinogenes, Tolumonas auensis DSM 9187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U 112, Francisella tularensis holarctica, Francisella tularensis FSC 198, Francisella tularensis, Francisella tularensis WY96-3418, or Treponema denticola (ATCC 35405).

In some embodiments, the nucleic acid sequence of interest that encodes in some embodiments, at least one therapeutic and/or modulatory molecule, for example, at least one therapeutic protein, that may be in some embodiments a receptor molecule such as a CAR molecule and/or a TCR molecule (e.g. in a nucleic acid cassette and/or construct) may be inserted into the appropriate genomic locus using a site-specific recombinase/integrase. Thus, in some embodiments, the systems of the present disclosure may comprise and/or provide, in addition to, or instead of at least one nuclease, also at least one recombinase and/or integrase. In some embodiments, the at least one recombinase and/or integrase may be one of any one of PhiC31, HK022, Cre, Flp, and more. The recombinase and/or integrase may be encoded on a nucleic acid cassette, construct and/or vector such as a plasmid, a mini-circle or a viral vector. Alternatively, the mRNA coding for the recombinase/integrase may be delivered, or the recombinase/integrase may be delivered as a protein.

Still further, in some embodiments, the nuclease of the system of the present disclosure may be at least one homing nuclease. In more specific embodiments, such homing nuclease may be at least one member of the LAGLIDADG family of homing endonucleases.

In more specific embodiments, the at least one member of the LAGLIDADG family of homing endonucleases used for the system of the present disclosure, is endonuclease I-CreI, or an engineered derivative thereof. I-CreI ([Sussman Et al. (2004), J. Mol. Biol. 342: 31-41; Chames et. al. (2005), Nucleic Acids Res. 33: e178; Seligman et al. (2002), Nucleic Acids Res. 30: 3870-9, Arnould et al. (2006), J. Mol. Biol. 355: 443-58)], having the amino acid sequence as denoted by UniProtKB—P05725 (DNE1_CHLRE)), is a member of the LAGLIDADG (SEQ ID NO: 1) family which recognizes and cleaves a 22 base pair recognition sequence in the chloroplast chromosome, and which presents an attractive target for meganuclease redesign. Genetic selection techniques have been used to modify the wild-type I-CreI recognition site preference. More recently, a method of rationally designing mono-LAGLIDADG (SEQ ID NO: 1) meganucleases was described which is capable of comprehensively redesigning I-CreI and other such meganucleases to target widely divergent DNA sites, including sites in mammalian, yeast, plant, bacterial, and viral genomes. The DNA sequences recognized by I-CreI are 22 base pairs in length, but the enzyme will bind to a variety of related sequences with varying affinity. The enzyme binds DNA as a homodimer in which each monomer makes direct contacts with a nine-base pair “half-site” and the two half-sites are separated by four base pairs that are not directly contacted by the enzyme. Like all LAGLIDADG (SEQ ID NO: 1) family meganucleases, I-CreI produces a staggered double-strand break at the center of its recognition sequences which results in the production of a four base pair 3-overhang. Thus, as used herein, the term “I-CreI-derived Meganuclease” refers to a rationally designed (i.e., genetically engineered) Meganuclease that is derived from I-CreI. The term genetically engineered Meganuclease, as used herein, refers to a recombinant variant of an I-CreI homing endonuclease that has been modified by one or more amino acid insertions, deletions or substitutions that affect one or more of DNA-binding specificity, DNA cleavage activity, DNA binding affinity, and/or dimerization properties.

A meganuclease may bind to double-stranded DNA as a homodimer, as is the case for wild-type I-CreI, or it may bind to DNA as a heterodimer. A Meganuclease may also be a “single-chain heterodimer” in which a pair of DNA-binding domains derived from I-CreI are joined into a single polypeptide using a peptide linker. The term “homing endonuclease” is synonymous with the term “Meganuclease.” As used herein, the term “rationally designed” means non-naturally occurring and/or genetically engineered.

As used herein, the term “rationally designed” means non-naturally occurring and/or genetically engineered, that differ from wild-type or naturally occurring meganucleases in their amino acid sequence or primary structure, and may also differ in their secondary, tertiary or quaternary structure. In addition, the rationally designed meganucleases of the invention also differ from wild-type or naturally occurring Meganucleases.

The at least one nuclease provided by the systems and/or kits of the present disclosure may be encoded on a nucleic acid cassette, construct and/or vector such as a plasmid, a mini-circle or a viral vector. Alternatively, the mRNA coding for at least one nuclease may be delivered, or the nuclease may be delivered as a protein. In some embodiments, the nuclease may be provided in at least one encoding nucleic acid sequence. In some embodiments, the nucleic acid sequence encoding at least nuclease may be provided by the disclosed systems and/or kits in the same vector and/or the same nucleic acid molecule, and/or cassette, together with the at least one nucleic acid sequence of interest. In yet some further embodiments, the nucleic acid sequence encoding at least nuclease may be provided by the disclosed systems and/or kits in separate vectors and/or separate nucleic acid molecule, and/or cassette/s.

In some particular and non-limiting embodiments, the engineered derivative of endonuclease I-CreI used for the system of the present disclosure, may be the ARCUS endonuclease that targets the TRAC locus.

In some embodiments, the ARCUS endonuclease is used by the systems and/or kits, cassettes, vectors, delivery vehicles, compositions, methods and uses of the present disclosure. ARCUS is a next-generation genome editing platform exhibiting a broad range of in vivo and ex vivo editing capabilities, unparalleled target specificity, and optimal delivery dynamics. The backbone of the ARCUS technology is the ARC Nuclease, a fully synthetic enzyme derived from the natural homing endonuclease I-CreI. Homing endonucleases are unique in their ability to specifically recognize very long DNA sequences (12-40 base pairs), statistically reducing the number of homologous target sites on the human genome to one. Because they were evolved to edit natural genomes, homing endonucleases have specialist mechanisms for recognizing and cutting DNA that eliminate off-target effects that could be toxic to the cell. Hence, the ARCUS platform is exquisitely specific. Moreover, binding of the endonuclease to its target generates a double-strand DNA break that triggers the cell's DNA repair pathways and stimulates efficient homologous recombination machinery. The ARC endonuclease is small in size and is encoded by a single gene and is therefore easy to deliver to cells. Moreover, ARCUS is much more programmable than previous homing-endonuclease based gene editing technologies, as it can be designed to recognize extremely diverse DNA sequences. Existing approaches rely largely on the ex vivo genetic engineering of a patient's T cells to express a tumor-specific chimeric antigen receptor (CAR) or a recombinant T cell receptor (rTCR) and the reintroduction of the engineered cells back into the patient. Although the technology has been highly successful on an individual-patient level, wider implementation has been hampered by the challenge of developing CAR/rTCR T cells for use in patients who lack sufficient numbers of high-quality T cells. In some embodiments, the ARCUS useful as a nuclease in the systems, kits, compositions and methods of the present disclosure may be any ARCUS designed to specifically target the TCR loci. Specifically, an ARCUS designed to specifically target the T cell receptor (TCR) α chain locus, or an ARCUS designed to specifically target the TCRβ chain locus, an ARCUS designed to specifically target the TCRγ chain locus, or an ARCUS designed to specifically target the TCRδ chain locus.

In some embodiments, ARCUS nuclease applicable in the systems, methods and compositions of the present disclosure may be an ARCUS designed to specifically target the one of the TCR alpha constant (TRAC), or an ARCUS designed to specifically target the TCR beta constant (TRBC) loci. In some embodiments, the ARCUS applicable in the disclosed systems, methods and compositions may be an ARCUS designed to specifically target the TRAC locus. In some specific and non-limiting embodiments, an ARCUS useful in the present disclosure may comprise the amino acid sequence as denoted by SEQ ID NO: 7, or any homologous, variants and derivatives thereof. Still further, in some embodiments, the ARCUS used in the present disclosure may be encoded by a nucleic acid sequence comprising the nucleic acid sequence of SEQ ID NO: 6, or any variants, homologs and derivatives thereof. Thus, in some embodiments, the systems of the present disclosure may comprise nucleic acid sequence that encodes ARCUS as disclosed herein and therefore may comprise the nucleic acid sequence as denoted by SEQ ID NO: 6, or any variants or homologs thereof. In some embodiments, the nucleic acid sequence encoding the disclosed ARCUS may be comprised within the same nucleic acid cassette, and/or construct that also comprise the nucleic acid sequence of interest that encodes the protein of interest, that may be in some embodiments and therapeutic protein, for example, a receptor molecule such as a CAR or engineered TCR.

Still further, in some embodiments, the target sequence recognized by the ARCUS used by the preset disclosure may be any target sequence within the TRAC and/or TRBC loci. In more specific embodiments, the target locus resides within the TRAC locus. In more specific embodiments, the target locus comprises a sequence recognized by the ARCUS used herein.

In some embodiments, the recognition sequence for the nuclease provided by the systems and/or kits of the present disclosure may be in some particular and non-limiting embodiments, within any exon or intron of the TRAC gene. In yet some further embodiments, the target site may be within exon 1 of TRAC gene.

In some embodiments, the recognition sequence comprises the nucleic acid sequence as denoted by SEQ ID NO: 8, or any variant thereof.

In some embodiments, the nucleic acid sequence of interest provided in the nucleic acid molecule of the systems and/or kits of the present disclosure, may comprise coding sequences and thus may comprise exons or fragments thereof that encode any product, for example, a protein or an enzyme (or fragments thereof). In other embodiments, the target nucleic acid sequence of interest may comprise non-coding sequences, as for example start codons, 5′ un-translated regions (5′ UTR), 3′ un-translated regions (3′ UTR), or other regulatory sequences, in particular regulatory sequences that are capable of increasing or decreasing the expression of specific genes within an organism. By way of example, regulatory sequences may be selected from, but are not limited to, transcription factors, activators, repressors and promoters. In further embodiments, the nucleic acid sequence of interest may comprise a combination of coding and non-coding regions.

In some embodiments, the nucleic acid sequence of interest may encode any product. In some embodiments the product may be a protein product. In yet some alternative embodiments, the product may be a nucleic acid product, for example, at least one inhibitory and/or modulatory non-coding nucleic acid molecule. In some embodiments, such inhibitory and/or modulatory non-coding nucleic acid molecule may be a ribonucleic acid (RNA) molecule. In yet some further specific embodiments, the RNA molecule may be at least one of a double-stranded RNA (dsRNA), an antisense RNA, a single-stranded RNA (ssRNA), and a Ribozyme. In some embodiments, the oligonucleotide aptamers and ODNs may be also applicable. In more specific embodiments, the at least one inhibitory and/or modulatory non-coding nucleic acid molecule encoded by the nucleic acid molecule of interest disclosed by the system of the invention, may be at least one of a microRNA (miRNA), MicroRNA-like RNAs (milRNA), artificial miRNAs (amiRNA), small interfering RNA (siRNA) and short hairpin RNA (shRNA).

Still further, in some embodiments the nucleic acid molecule of interest may encode at least one therapeutic protein. Examples of therapeutic proteins include receptors, ligands, immune checkpoint molecules, antibodies, growth factors, clotting factors, and/or cytokines, or any combinations thereof.

It should be appreciated that the disclosed nucleic acid sequence of interest may further encode, in addition to the therapeutic product, any other affinity molecule that may be served as a molecular tag. Non-limiting examples for such tags may include His-tag, Flag, HA, myc, Halo tags, GFP, and the like.

Thus, in some specific embodiments, the at least one exogenous nucleic acid sequence of interest may encode at least one therapeutic molecule, e.g., at least one receptor molecule. In some embodiments, such for example, at least one of: at least one chimeric antigen receptor (CAR) and/or at least one exogenous and/or engineered TCR, or parts thereof. In some optional embodiments, in addition to the disclosed CAR and/or TCR, the exogenous nucleic acid sequence may further comprise sequences encoding at least one additional effect or, for example, at least one cytokine, chemokine and the like.

The nucleic acid sequence of interest comprised within the cassette of the present disclosure, used and provided by the systems, methods and compositions disclosed herein may encode at least one therapeutic protein or element (e.g., any ribonucleic acid molecule displaying a director indirect therapeutic effect, for example miRNA, siRNA and the like). In some particular embodiments, the nucleic acid sequence of interest comprised within the cassette of the present disclosure, used and provided by the systems, methods and compositions disclosed herein may encode at least one receptor. A receptor molecule as used herein refer to a molecular structure or site, specifically a protein on the surface or interior of a cell that binds ligand substances such as hormones, antigens, drugs, or neurotransmitters. In more specific embodiments such receptor may be at least one of a T cell receptor (TCR) and a chimeric antigen receptor (CAR), or any combinations thereof.

It should be noted that “receptor” as used herein also encompasses any variant, chimeric or fusion protein of any of the receptors described herein, specifically, TCR or BCR chimeric or fusion proteins.

In some embodiments, the nucleic acid sequence of interest of the systems of the present disclosure encodes at least one CAR molecule. Thus, in some embodiments, the engineered receptor molecules encoded by the nucleic acid sequence of interest, e.g., the CAR and/or exogeneous or engineered TCR molecules, may be specifically directed against at least one antigen associated with at least one pathological disorder. In some embodiments, the pathological disorder may be any immune-related disorder as disclosed herein after. Still further, in some embodiments, the CAR and/or TCR molecules may be specifically directed against at least one pathogenic entity associated with an immune-related disorder, for example, a proliferative disorder (e.g., cancer), an infectious disease caused by at least one pathogen (e.g., viral, bacterial, fungal pathogen) and the like. In some embodiments thereof, the disclosed CAR and/or exogeneous and/or engineered TCR molecules may specifically recognize an antigen associated with, or specific for a proliferative disorder. In particular, the disclosed in vivo engineering systems and methods disclosed herein may provide activation and expansion of T cells that target tumor associated antigens (TAA). Relevant TAAs applicable in the present disclosure are disclosed herein after. Naturally occurring T cell receptors (TCRs) often have low affinity against TAAs. However, T cells can be in vivo engineered by the disclosed systems and methods to express highly potent TCRs, which can recognize fragments of both intracellular and cell surface proteins when presented in a major histocompatibility complex (MHC) context. Alternatively, as indicated herein, T cells can be in vivo engineered to express chimeric antigen receptors (CARs), which have a high TAA affinity. As used herein, the term “fusion or chimeric protein” refers to a recombinant protein in which two or more proteins or domains responsible for a specific function within a protein are linked so that each protein or domain is responsible for its intrinsic function. A linker having a flexible structure may conventionally be inserted between the two or more proteins or domains.

In yet some further embodiments, the protein encoded by the nucleic acid sequence comprised within the cassette provided by the present disclosure and by the systems and/or kits, compositions, methods and uses of the present disclosure, may be a T-cell receptor (TCR). The TCR is a disulfide-linked membrane-anchored heterodimeric protein normally consisting of the highly variable alpha (α) and beta (β) chains expressed as part of a complex with the invariant CD3 chain molecules. T cells expressing this receptor are referred to as α:β (or αβ) T cells, though a minority of T cells express an alternate receptor, formed by variable gamma (γ) and delta (δ) chains, referred as γδ T cells. Each chain is composed of two extracellular domains: Variable (V) region and a Constant (C) region. The variable domain of both the TCR α-chain and β-chain each have three hypervariable or complementarity determining regions (CDRs), as well as framework regions (FRs) and a constant region. The sequence diversity of a beta T cells is largely determined by the amino acid sequence of the third complementarity-determining region (CDR3) loops of the α and β chain variable domains, which diversity is a result of V(D)J recombination.

It should be understood that in case the nucleic acid sequence of interest provided by the systems and/or kits, as well as the compositions and methods of the present disclosure, encodes at least one TCR, the TRC may be in some embodiments a recombinant TCR directed against at least one antigen.

As indicated above, in some further embodiments, the protein encoded by the nucleic acid sequence comprised within the system/s and/or kit/s, cassette/s and/or compositions provided by the present disclosure may be a Chimeric antigen receptor (CAR). CAR, as used herein, relates to artificial T cell receptors (also known as chimeric T cell receptors, chimeric immuno-receptors). These are engineered receptors, which graft an arbitrary specificity onto an immune effector cell. Typically, these receptors are used to graft the specificity of a monoclonal antibody onto a T cell.

The initial design (also referred to a first generation) joined an antibody-derived scFv to the CD3ζ intracellular signaling domain of the T-cell receptor through hinge and transmembrane domains.

Second generation CARs add intracellular signaling domains from various costimulatory protein receptors (e.g., CD28, 41BB, ICOS) to the cytoplasmic tail of the CAR to provide additional signals to the T cell. More recently, third generation CARs combine multiple signaling domains, such as CD27, CD28, 4-1BB, ICOS, or OX40, to augment potency. Still further, in some embodiments, a fourth generation CAR T molecule may be used in the present disclosure as encoded by the exogenous nucleic acid sequence of interest, that is in vivo targeted to the target genomic locus within the T cells as discussed by the present disclosure. More specifically, such TRUCKs (“T cells redirected for antigen-unrestricted cytokine-initiated killing”), also called “4th generation” CAR T cells, combine the direct antitumor attack of the CAR T cell with the immune modulating capacities of the delivered cytokine. Through CAR-induced release, the cytokine is ideally deposited in the targeted tissue alleviating systemic side effects. The TRUCK concept is currently being explored using a panel of cytokines, including IL-2, IL-7, IL-12, IL-15, IL-18, IL-23, IL27, and combinations thereof. Thus, in some embodiments, the exogenous nucleic acid sequence of interest may further encode for additional effectors, specifically, cytokines, and chemokines.

The CAR molecule encoded by the nucleic acids sequence of interest targeted to the TRAC and/or TRBC loci, provided by the systems and/or kits, compositions, methods and uses of the present disclosure, may comprise at least one antigen binding domain. Exemplary categories of antigen-binding domains that can be used in the context of the present invention include antibodies, antigen-binding portions of antibodies, peptides that specifically interact with a particular antigen (e.g., peptibodies), receptor molecules that specifically interact with a particular antigen, proteins comprising a ligand-binding portion of a receptor that specifically binds a particular antigen or antigen-binding scaffold, or even peptide aptamers. The antigen binding domains in accordance with the present disclosure may recognize and bind a specific antigen or epitope. It should be therefore noted that the term “binding specificity”, “specifically binds to an antigen”, “specifically immuno-reactive with”, “specifically directed against” or “specifically recognizes”, when referring to an antigen or particular epitope, refers to a binding reaction which is determinative of the presence of the epitope in a heterogeneous population of proteins and other biologics. The term “epitope” is meant to refer to that portion of any molecule capable of being bound by the affinity molecule, specifically, an antibody which can also be recognized by that antibody. Epitopes or “antigenic determinants” usually consist of chemically active surface groupings of molecules such as amino acids or sugar side chains and have specific three-dimensional structural characteristics as well as specific charge characteristics. Still further, as indicated above, an “antigen-binding domain” can in some embodiments, comprise or consist of an antibody or antigen-binding fragment of an antibody. The term “antibody” as used herein, means any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

The antibody suitable for the present disclosure may also be a bi-specific antibody (such as Bi-specific T-cell engagers-BiTEs) or a tri-specific antibody.

The antibody suitable for the invention may also be a variable new antigen receptor antibody (V-NAR). VNARs are a class of small, immunoglobulin-like molecules from the shark immune system. Humanized versions of VNARs could be used to bind protein epitopes that are difficult to access using traditional antibodies.

Still further, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

Still further, in some embodiments, the target-recognition domain of the CAR molecule and/or the TCR molecule, used herein as the desired product encoded by the nucleic acid sequence of interest of the present disclosure is part of the extracellular domain of the molecule. In yet some further embodiments, the extracellular domain of the CAR molecule of the present disclosure may comprise at least one additional component or domains. More specifically, further components that may be added to the extracellular domain may comprise, for example, at least one spacer domain comprising at least one tag or labeling moiety. Such labeling moiety may comprise for example, Strep-II, HA-tag, Flag, myc-tag, and the like. In yet some further embodiments, the extracellular domain of the disclosed CAR molecule may comprise at least one hinge region. The hinge region, as used herein, is a flexible amino acid stretch/structure, which may in some embodiments link between the target-binding domain and the transmembrane domain of the disclosed CAR molecule. In some embodiments, the hinge-region may be rich in cysteine and proline amino acids. In some embodiments, hinge regions useful in the present invention are generally derived from IgG subclasses (such as IgG1 and IgG4), IgD and CD8 domains, of which IgG1 has been most extensively used. In yet some further embodiments, hinge regions may be derived from the CD8α molecule or the CD28 molecule.

In yet some further embodiments, the second component of the disclosed CAR molecules may be at least one transmembrane domain. A transmembrane domain (TMD) is a membrane-spanning domain that may traverse the membrane bilayer once or several times. TMDs may consist predominantly of nonpolar amino acid residues and generally adopt an alpha helix conformation.

Still further, the third component of the CAR molecule of the present disclosure may be at least one signal transduction domain. According to some embodiments, this domain is an intracellular domain connected to the transmembrane domain.

Still further, in some embodiments, the at least one intracellular T cell signal transduction domain of the of the CAR-molecule in accordance with the present disclosure, may comprise sequences derived from at least one tumor necrosis factor (TNF) receptor family member, specifically, the 4-1BB. In yet some further optional embodiments, the at least one intracellular T cell signal transduction domain of the of the CAR-molecule of the present disclosure, further comprises at least one TCR molecule or any fragments thereof. In more specific embodiments, such domain is derived from the cluster of differentiation 3 (CD3) zeta chain or crystallizable fragment receptor gamma.

It should be noted that in some specific embodiments, the TCR and/or CAR that may be in some embodiments, encoded by the nucleic acid sequence of interest of the system/s and/or kit/s of the present disclosure may be directed at or specific for a tumor associated antigen (TAA).

Tumor or cancer associated antigen, as used herein may be an antigen that is specifically expressed, over expressed or differentially expressed in tumor cells. In yet some further embodiments, TAA can stimulate tumor-specific T-cell immune responses. Exemplary tumor antigens that may be applicable in the present invention, include, but are not limited to, RAGE-1, tyrosinase, MAGE-1, MAGE-2, NY-ESO-1, Melan-A/MART-1, glycoprotein (gp) 75, gp100, MUC1, beta-catenin, PRAME, MUM-1, WT-1, CEA, PR-1 CD45, glypican-3, IGF2B3, Kallikrein4, KIF20A, Lengsin, Meloe, MUC5AC, survivin, CLPP, Cyclin-A1, SSX2, XAGE1b/GAGED2a, MAGE-A3, MAGE-A6, LAGE-1, CAMEL, hTRT and Eph. and TRP-1. Still further, TAA may be recognized by CD8+ T cells as well as CD4+ T cells. Non limiting examples of TAA recognized by CD8+ T cells may be CSNKIAI, GAS7, HAUS3, PLEKHM2, PPPIR3B, MATN2, CDK2, SRPX (P55L), WDR46 (T227I), AHNAK (54460F), COL18A1 (S126F), ERBB2 (H197Y), TEAD1 (L209F), NSDHL (A290V), GANAB (S184F), TRIP12 (F1544S), TKT (R438W), CDKN2A (E153K), TMEM48 (F169L), AKAP13 (Q285K), SEC24A (P469L), OR8B3 (T190I), EXOC8 (Q656P), MRPS5 (P59L), PABPCI (R520Q), MLL2, ASTN1, CDK4, GNL3L, SMARCD3, MAGE-A6, MED13, PASSA WDR46, HELZ2, AFMID, CENPL, PRDX3, FLNA, KIF16B, SON, MTFR2 (D626Y), CHTF18 (L769V), MYADM (R30W), NUP98 (A359D), KRAS (G12D), CASP8 (F67V), TUBGCP2 (P293L), RNF213 (N1702S), SKIV2L (R653H), H3F3B (A48T), AP15 (R243Q), RNF10 (E572K), PHLPPI (G566E) and ZFYVE27 (R6H). Non limiting examples of TAA recognized by CD4+ T cells may be ERBB2IP (E805G), CIRHIA (P333L), GART (V551A), ASAP1 (P941L), RND3 (P49S), LEMD2 (P495L), TNIK (S502F), RPS12 (V104I), ZC3H18 (G269R), GPD2 (E426K), PLEC (E1179K), XPO7 (P274S), AKAP2 (Q418K) and ITGB4 (510021). Non-limiting examples of MHC class II-restricted antigens may be Tyrosinase, gp100, MART-1, MAGE-A1, MAGE-A2, MAGE-A3, MAGE-A6, LAGE-1, CAMEL, NY-ESO-1, hTRT and Eph.

Cancer antigens and tumor antigens are used interchangeably herein. The antigens applicable as targets for the CAR and/or TCR molecules encoded as a therapeutic product by the nucleic acid molecules provided by the disclosed system/s and/or kit/s, may be related to cancers that include, but are not limited to, Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer).

In some specific and non-limiting embodiments, the nucleic acid sequence of interest may encode a CAR molecule specific for the CD19 antigen, also denoted herein as CD19-CAR-T. Still further, CD19 is a B-cell surface protein expressed throughout B-cell development. Therefore, it is expressed in nearly all B-cell malignancies, including chronic lymphocytic leukemia (CLL), ALL, and many non-Hodgkin lymphomas. This near-universal expression and specificity for a single cell lineage has made CD19 an attractive target for CAR-modified T-cell therapies. Additional B-cell-specific cell-surface molecules, such as CD22, may hold similar promises and are under active investigation, and may therefore be also applicable in the present disclosure.

Thus, in some particular and non-limiting embodiments, the in vivo targeting systems and/or kits, compositions, methods and uses of the present disclosure, may be employed to target an anti CD19 CAR coding nucleic acid sequence which are highly effective in treating B cell malignancies. In yet some further specific embodiments, in vivo targeting according to the invention may be employed to target the nucleic acid sequence that encodes the anti CD19 CAR, specifically, such anti-CD19 CAR may comprise in some embodiments, the amino acid sequence as denoted by SEQ ID NO: 4, or any homologs, variants or derivatives thereof. Thus, in some embodiments, the nucleic acid molecule, cassettes provided by the disclosed systems and used by the methods and compositions discussed herein may comprise as at least one nucleic acid sequence of interest, a nucleic acid sequence encoding the polypeptide as denoted by SEQ ID NO. 5, or any derivatives or variants thereof, encoding the anti-human CD19 CAR molecule.

In some specific embodiments, the CAR molecule encoded by the nucleic acid sequence of interest of the system of the present disclosure may be an anti CD19 CAR molecule. In some specific and non-limiting embodiments, the anti-CD19 CAR used herein may comprise the amino acid sequence as denoted by SEQ ID NO: 4, or any derivatives or variants thereof. Thus, in some embodiments, the system of the present disclosure may comprise as component (a), a nucleic acid molecule comprising at least one nucleic acid sequence encoding the anti-CD19 CAR of SEQ ID NO: 4, or any derivatives or variants thereof. In more specific embodiments, the nucleic acid molecule of the disclosed systems may comprise at least one nucleic acid sequence that may comprise SEQ ID NO: 5, or any derivatives, homologs and variants thereof.

In some embodiments, the nucleic acid sequence of interest encodes at least one exogenous molecule of interest, for example, any therapeutic molecule, more specifically, at least one receptor molecule such as CAR molecule/s and/or an exogeneous and/or engineered TCR molecule/s. In yet some further embodiments, the targeted insertion of the exogenous molecule of interest, specifically at least one therapeutic molecule such as TCR and/or CAR into the TRAC and/or TRBC loci, disrupts or at least reduces the expression of the endogenous TCR by the T cells. Thus, in some embodiments, the in vivo targeted insertion of the disclosed nucleic acid sequences of interest provided by the disclosed systems decrease, inhibits, reduces, attenuates the expression of the endogenous TCR molecule of the cell. Such reduction, or “decrease” as referred to herein, relate to the reduction of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more, as compared with the expression of the endogenous TCR molecule in T cells that did not undergo targeted insertion of at least one nucleic acid molecule of interest into a target locus.

In yet some alternative embodiments, the CAR or TCR encoding nucleic acid sequences of the nucleic acid molecules of the disclosed systems, may be preceded by a splice acceptor followed by a 2A peptide or an IRES sequence, thereby allowing the CAR or TCR to be transcribed together with the endogenous variable domains of the TCR chains. The CAR or TCR is separated from the variable domains of the endogenous TCR chains upon translation due to the polycistronic coding facilitated by the 2A peptide or IRES sequences.

It should be understood that in some embodiments, each of the nucleic acid molecules provided by the disclosed systems and/or kits, and/or the nucleic acid sequence encoding the at least one nuclease, may be provided in at least one cassette, vector and/or vehicle.

Still further, in some embodiments, the at least one nucleic acid molecule comprising the at least one exogenous nucleic acid sequence of interest (defined herein as component (a) of the system of the present disclosure) and the nucleic acid sequence encoding at least one site specific nuclease (defined herein as component (b) of the system of the present disclosure) may be comprised within the same cassette/s, and/or constructs, and/or vector/s. It should be noted that the vector is at least one AAV vector and/or AAV-like vector.

In yet some alternative embodiments, the at least one nucleic acid molecule comprising the at least one exogenous nucleic acid sequence of interest (component (a)), and the nucleic acid sequence encoding at least one site specific nuclease (component (b)), are provided in separate cassette/s and/or construct/s, and/or vector/s and/or delivery vehicle/s. It should be noted that the at least one nucleic acid molecule and/or at least one cassette/s and/or construct/s thereof, is comprised within at least one AAV vector and/or AAV-like vector.

Still further, in some embodiments, the nuclease may be provided in a separate vector, it can be in some embodiments provided as mRNA or a modified mRNA, coding for the nuclease. Alternatively, the nuclease may be provided in the disclosed system as a protein. Such protein may be provided in a separate cassette/s and/or construct/s, and/or vector/s and/or delivery vehicle/s.

In some embodiments, the nucleic acid molecule/s of the disclosed systems or any cassette/s, vector/s or vehicle/s thereof, provided by the system of the present disclosure, may further comprise at least one genetic element, said genetic element is at least one of: an internal ribosome entry site (IRES), a 2A peptide coding sequence, a promoter or any functional fragments thereof, a splice donor (SD), a splice acceptor (SA), a degron, a 3 frame stop, a protein stabilizing sequence, a signal peptide, a stop codon, a polyadenylation site, a transcription enhancer, a switch region, an mRNA stabilizing sequence and a protein stabilizing sequence.

In some embodiments, the desired molecule of interest, e.g., the CAR and/or the TCR coding nucleic acid sequences provided in the disclosed systems may be integrated with a promoter, which may be T cell specific, and may further be active only upon genomic integration of the CAR or TCR cassette, as then it is placed in proximity to the necessary enhancers of the TRAC and/or TRBC loci.

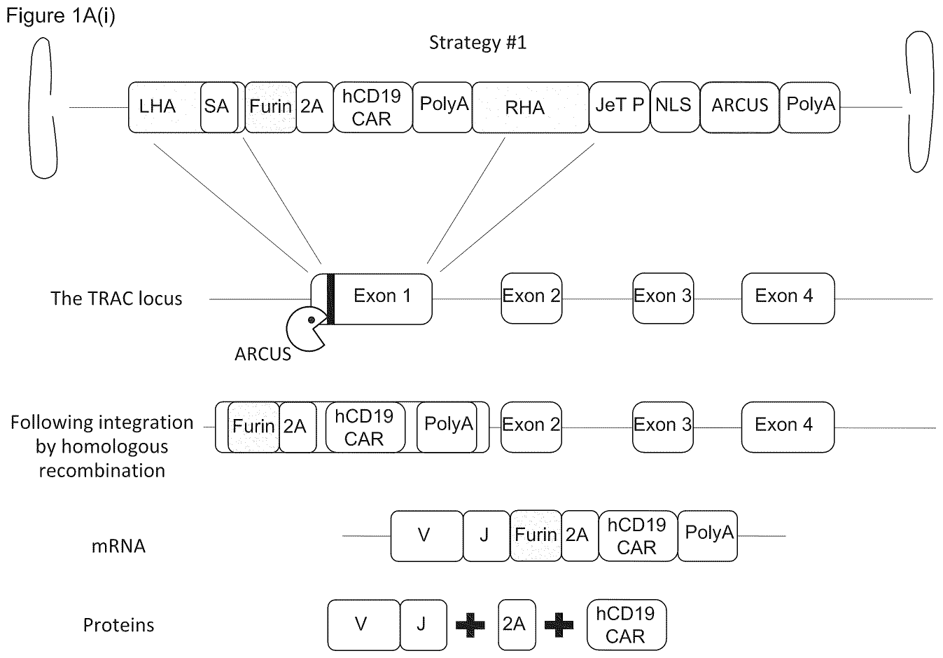

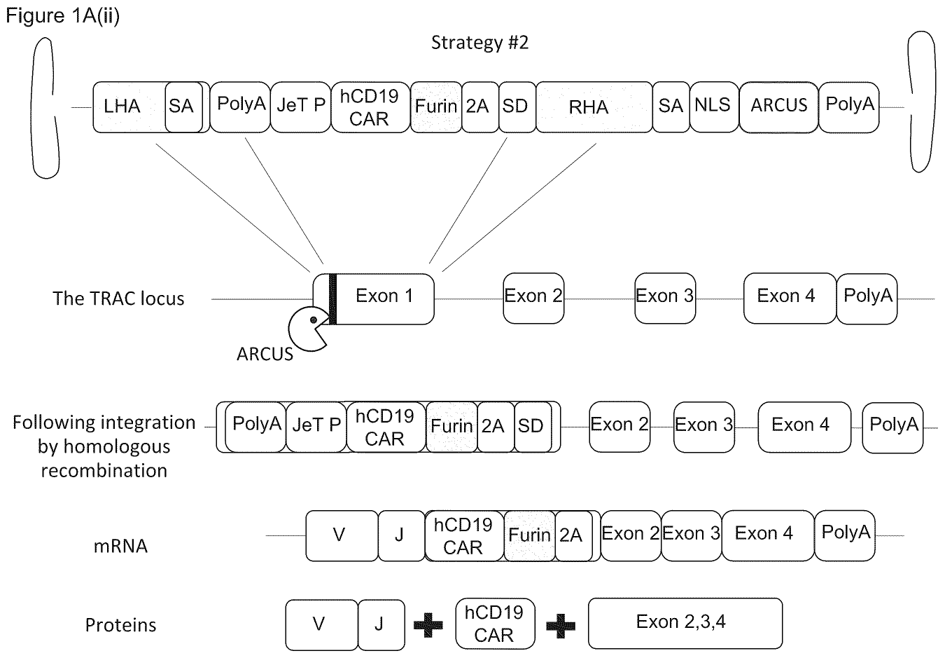

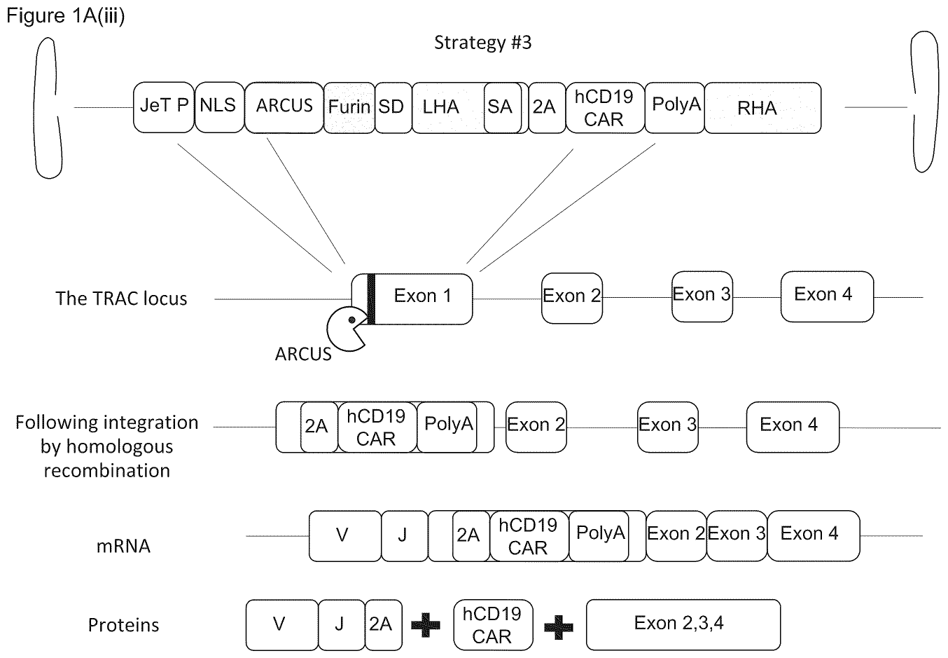

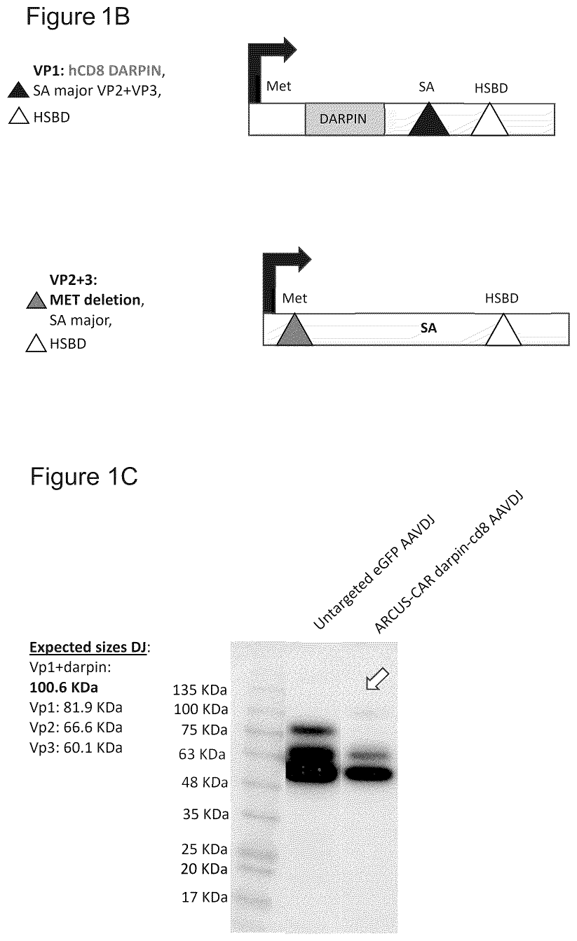

In some specific and non-limiting embodiments, such systems may comprise the nucleic acid molecule, e.g., the cassette as discussed in strategy I of the present disclosure, as also illustrated in FIG. 1A(i).

In yet some alternative embodiments, the nucleic acid sequence of interest, specifically, the CAR and/or the TCR coding nucleic acid sequences may be integrated with any appropriate promoter as disclosed herein that may be a non-T cell specific promoter.

In some specific and non-limiting embodiments, such systems may comprise the nucleic acid molecule, e.g., the cassette as discussed in strategy II and/or strategy III of the present disclosure, as also illustrated in FIG. 1A(ii) and (iii).

Thus, in some embodiments, the promoter/s used herein may be a T cell promoter, for example, endogenous T cell promoter, or non-endogenous T cell promoter.

As indicated above, in some further embodiments, the nucleic acid molecule of the disclosed systems and/or kits or any cassettes, constructs thereof, provided by the present disclosure and used in the systems, compositions and methods disclosed herein, may be comprised within a nucleic acid vector, and/or delivery vehicle/s. In more specific embodiments, such vector or delivery vehicle may be any one of a viral vector, a non-viral vector and a naked DNA vector.

Vectors, as used herein, are nucleic acid molecules of particular sequence can be incorporated into a vector that is then introduced into a host cell e.g., in vivo, as disclosed herein, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g. plasmids, cosmids, minicircles, phage, viruses, etc., useful for transferring nucleic acids into target cells may be applicable in the present disclosure. The vectors comprising the nucleic acid(s) may be maintained episomally, e.g. as plasmids, minicircle DNAs, viruses such cytomegalovirus, adenovirus, etc., or they may be integrated into the target cell genome, through homologous recombination or random integration, e.g. retrovirus-derived vectors such as AAV, MMLV, HIV-1, ALV, etc.

Vectors may be provided directly to the subject that comprise the target cells. In other words, the cells are contacted in vivo with vectors and/or delivery vehicles comprising the nucleic acid molecule of the disclosed systems and/or kits, or with any cassettes that comprise the nucleic acid sequence of interest. The vectors are taken up by the cells in vivo, in the body of the administered subject, or in any tissue or organs thereof. Nucleic acid molecules can be introduced in vivo to the target cells as naked nucleic acid, as nucleic acid complexed with an agent such as a liposome or poloxamer, or can be delivered by viruses (e.g., adenovirus, AAV), also indicated herein as a delivery vehicle or a viral delivery vehicle.

As indicated above, in some embodiments, viral vectors or delivery vehicles may be applicable in the present invention. The term “viral vector” refers to a replication competent or replication-deficient viral particle which are capable of transferring nucleic acid molecules into a host.

The term “virus” refers to any of the obligatory intracellular parasites having no protein-synthesizing or energy-generating mechanism. The viral genome may be RNA or DNA contained with a coated structure of protein of a lipid membrane. Examples of viruses useful in the practice of the present disclosure include baculoviridiae, parvoviridiae, picornoviridiae, herepesviridiae, poxviridiae, adenoviridiae, picotmaviridiae. The term recombinant virus includes chimeric (or even multimeric) viruses, i.e. vectors constructed using complementary coding sequences from more than one viral subtype. Still further, in some embodiments, a viral vector useful in the present disclosure may be any native viral delivery vehicle that was engineered to pack the nucleic acid molecules and/or cassettes thereof provided by the systems of the present disclosure that includes the nucleic acid sequence of interest, and/or nucleic acid sequence that encodes the at least one nuclease. However, it should be appreciated that the present disclosure further encompasses the use of any recombinant, genetically modified, and/or synthetic viral particles, and/or virus-like particles, and/or any transducing particles as delivery vehicles for the nucleic acid molecules of the present disclosure. Still further, in some embodiments, the viral delivery vehicles may be either native viral vectors, or modified and/or genetically engineered viral vectors, that may be further engineered to express on the capsid thereof, and/or targeting moieties that direct the delivery vector towards the target cells, and/or target tissue and/or target organ. Specific engineered viral vectors encompassed by the present disclosure are described herein after.

In more specific embodiments, the vector and/or delivery vehicle of the system of the present disclosure, may be at least one viral vector. More specifically, such at least one viral vector may be any one of adeno associated virus (AAV) vector, AAV-like vectors, recombinant adeno associated virus vectors (rAAV), single stranded AAV (ssAAV), self-complementary rAAV (scAAV), Simian vacuolating virus 40 (SV40) vector, Adeno virus vector, helper-dependent Adeno viral vector, retroviral vector and lentiviral vector.

In some embodiments, the nucleic acid molecule that comprise the nucleic acid sequence of interest, and/or nucleic acid sequence that encodes the at least one nuclease, and/or any cassette thereof of the systems disclosed herein, may be comprised within an Adeno-associated virus (AAV). The term “adenovirus” is synonymous with the term “adenoviral vector”. AAV is a single-stranded DNA virus with a small (˜20 nm) protein capsule that belongs to the family of parvoviridae, and specifically refers to viruses of the genus adenoviridiae. The term adenoviridiae refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof the individual serotypes and A-F subgenera including but not limited to human adenovirus types 1, 2, 3, 4, 4a, 5, 6, 7, 8, 9, 10, 11 (AdllA and Ad IIP), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91.

Due to its inability to replicate in the absence of helpervirus coinfections (typically Adenovirus or Herpesvirus infections) AAV is often referred to as dependovirus. AAV infections produce only mild immune responses and are considered to be nonpathogenic, a fact that is also reflected by lowered biosafety level requirements for the work with recombinant AAVs (rAAV) compared to other popular viral vector systems. Due to its low immunogenicity and the absence of cytotoxic response AAV-based expression systems offer the possibility to express genes of interest for months in quiescent cells. Production systems for rAAV vectors typically consist of a DNA-based vector containing a transgene expression cassette, which is flanked by inverted terminal repeats (ITR). Construct sizes are limited to approximately 4.7-5.0 kb, which corresponds to the length of the wild-type AAV genome. rAAVs are produced in cell lines. The expression vector is co-transfected with a helper plasmid that mediates expression of the AAV rep genes which are important for virus replication and cap genes that encode the proteins forming the capsid. Recombinant adeno-associated viral vectors can transduce dividing and non-dividing cells, and different rAAV serotypes may transduce diverse cell types. These single-stranded DNA viral vectors have high transduction rates and have a unique property of stimulating endogenous Homologous Recombination without causing double strand DNA breaks in the host genome.

It should be appreciated that many intermediate steps of the wild-type infection cycle of AAV depend on specific interactions of the capsid proteins with the infected cell. These interactions are crucial determinants of efficient transduction and expression of genes of interest when rAAV is used as gene delivery tool. Indeed, significant differences in transduction efficacy of various serotypes for particular tissues and cell types have been described. Thus, in some embodiments, AAV serotype 6 may be suitable for the systems and/or kits, compositions, methods and uses of the present disclosure. In yet some further embodiments, AAV serotype 8 may be suitable for the methods of the invention. In some embodiments, the AAV serotype 6 may be encoded by the nucleic acid sequence as denoted by GenBank accession number AF028704.1.

In some embodiments, the AAV vector provided and used by the present disclosure may be the AAV-DJ system. In some embodiments, the AAV-DJ system provides a hybrid capsid created by DNA shuffling technology combining 8 different native serotypes: AAV-2, AAV-4, AAV-5, AAV-8, AAV-9, avian AAV, bovine AAV, and caprine AAV. The resulting AAV vector is a highly infectious vector that can transduce a wide variety of cells and tissues at significantly higher rates than AAV-2. In yet some further embodiments, the AAV DJ vector used in the present disclosure is able transducing primary T cells as also described by (J. Virol. 2008 June; 82(12):5887-5911). It should be understood that in some embodiments of the present disclosure the AAV-DJ serotype is used for each of the disclosed aspects.

It is believed that a rate-limiting step for the AAV-mediated expression of transgenes is the formation of double-stranded DNA. Recent reports demonstrated the usage of rAAV constructs with a self-complementing structure (scAAV) in which the two halves of the single-stranded AAV genome can form an intra-molecular double-strand. This approach reduces the effective genome size usable for gene delivery to about 2.3 kB but leads to significantly shortened onsets of expression in comparison with conventional single-stranded AAV expression constructs (ssAAV). Thus, in some embodiments, ssAAV may be applicable as a viral vector by the systems and/or kits, compositions, methods and uses of the present disclosure.

In yet some further embodiments, HDAd vectors may be suitable for the systems and/or kits, compositions, methods and uses of the present disclosure. The Helper-Dependent Adenoviral (HDAd) vectors HDAds have innovative features including the complete absence of viral coding sequences and the ability to mediate high level transgene expression with negligible chronic toxicity. HDAds are constructed by removing all viral sequences from the adenoviral vector genome except the packaging sequence and inverted terminal repeats, thereby eliminating the issue of residual viral gene expression associated with early generation adenoviral vectors. HDAds can mediate high efficiency transduction, do not integrate in the host genome, and have a large cloning capacity of up to 37 kb, which allows for the delivery of multiple transgenes or entire genomic loci, or large cis-acting elements to enhance or regulate tissue-specific transgene expression. One of the most attractive features of HDAd vectors is the long term expression of the transgene.

Still further, in some embodiments, SV40 may be used as a suitable vector by the systems and/or kits, compositions, methods and uses of the present disclosure. SV40 vectors (SV40) are vectors originating from modifications brought to Simian virus-40 an icosahedral papovavirus. Recombinant SV40 vectors are good candidates for gene transfer, as they display some unique features: SV40 is a well-known virus, non-replicative vectors are easy-to-make, and can be produced in titers of 10(12) IU/ml. They also efficiently transduce both resting and dividing cells, deliver persistent transgene expression to a wide range of cell types, and are non-immunogenic. Present disadvantages of rSV40 vectors for gene therapy are a small cloning capacity and the possible risks related to random integration of the viral genome into the host genome.

In certain embodiments, an appropriate vector that may be used by the systems and/or kits, compositions, methods and uses of the present disclosure may be a retroviral vector. A retroviral vector consists of proviral sequences that can accommodate the gene of interest, to allow incorporation of both into the target cells. The vector may also contain viral and cellular gene promoters, to enhance expression of the gene of interest in the target cells. Retroviral vectors stably integrate into the dividing target cell genome so that the introduced gene is passed on and expressed in all daughter cells. They contain a reverse transcriptase that allows integration into the host genome.

In yet some alternative embodiments, lentiviral vectors may be used in the systems and/or kits, compositions, methods and uses of the present disclosure. Lentiviral vectors are derived from lentiviruses which are a subclass of Retroviruses. Commonly used retroviral vectors are “defective”, i.e. unable to produce viral proteins required for productive infection. Rather, replication of the vector requires growth in a packaging cell line. To generate viral particles comprising the cassette with the nucleic acids sequence of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the cassette of the invention that contains the nucleic acids sequence of interest into packaging cell lines and of collecting the viral particles that are generated by the packaging lines are well known in the art.

In some alternative embodiments, the vector used in the systems and/or kits, compositions, methods and uses of the present disclosure, may be a non-viral vector. More specifically, such vectors may be in some embodiments any one of plasmid, minicircle and linear DNA.

Nonviral vectors, in accordance with the invention, refer to all the physical and chemical systems except viral systems and generally include either chemical methods, such as cationic liposomes and polymers, or physical methods, such as gene gun, electroporation, particle bombardment, ultrasound utilization, and magnetofection. Efficiency of this system is less than viral systems in gene transduction, but their cost-effectiveness, availability, and more importantly reduced induction of immune system and no limitation in size of transgenic DNA compared with viral system have made them attractive also for gene delivery.

For example, physical methods applied for in vitro and in vivo gene delivery are based on making transient penetration in cell membrane by mechanical, electrical, ultrasonic, hydrodynamic, or laser-based energy so that DNA entrance into the targeted cells is facilitated.

In more specific embodiments, the vector may be a naked DNA vector. More specifically, such vectors may be for example, a plasmid, minicircle or linear DNA.

Naked DNA alone may facilitate transfer of a gene (2-19 kb) into skin, thymus, cardiac muscle, and especially skeletal muscle and liver cells when directly injected. It enables also long-term expression. Although naked DNA injection is a safe and simple method, its efficiency for gene delivery is quite low.

Minicircles are modified plasmid in which a bacterial origin of replication (ori) was removed, and therefore they cannot replicate in bacteria.

Linear DNA or Doggybone™ are double-stranded, linear DNA construct that solely encodes an antigen expression cassette, comprising antigen, promoter, polyA tail and telomeric ends.

It should be appreciated that all DNA vectors disclosed herein may be also applicable in the cassettes, systems and/or kits, compositions, methods and uses of the present disclosure.

Still further, in some embodiments, the vector used by the system of the present disclosure may further comprise at least one T cell targeting moiety.

In some embodiments, the vector and/or delivery vehicle used by the disclosed systems for in vivo delivering the nucleic acid molecule of interest and/or the at least one nuclease and any nucleic acid sequence encoding the same, may be an AAV vector. In yet some further embodiments, the AAV vector used by the disclosed systems may be a modified and/or AAV vector. Still further, in some embodiments, the AAV vector used by the disclosed systems may be an AAV vector that comprises at least one targeting moiety. In some embodiments, such targeting moiety may be a T cell targeting moiety. It should be further understood that the viral vector disclosed herein, specifically, the AAV vector may comprise in some embodiments at least one targeting moiety, for example, T cell specific targeting moiety. The targeting moiety may be connected and/or associated directly or indirectly, e.g., via a linker and/or spacer, to the delivery vehicle used. In case of a viral vector, for example, AAV, at least one targeting moiety may be in some embodiments associated with and/or connected to, and/or incorporated into the capsid of the virus, in case of AAV, or to any viral envelop of any other viral vector used. In some specific embodiments, the at least one targeting moiety may be connected and/or associated to at least one of the viral capsid proteins, for example, at least one of VP1 and/or VP2, and/or VP3. In yet some other specific embodiments, the at least one targeting moiety may be incorporated into the viral capsid, for example, as part of at least one of the viral capsid protein/s. In yet some further embodiments, the at least one targeting moiety may be incorporated into the viral capsid by replacing at least one of the viral proteins. In some alternative embodiments, the at least one targeting moiety may be expressed as a fusion and/or chimera of at least one of the viral proteins, specifically, at least one of VP1, VP2 and/or VP3. Still further, in some specific embodiments, the targeting moiety may be incorporated into the VP1 capsid protein.

In yet some further embodiments, the AAV vector used in the disclosed systems may be an AAV vector expressing in its capsid, at least one T cell targeting moiety, incorporated therein.

In some embodiments, the targeting moiety used in the viral vectors of the present disclosure may be at least one DARPIN molecule. DARPIN, and specifically, “Designed Ankyrin Repeat Proteins”, are a class of artificial proteins that are engineered to bind with high specificity to target molecules, such as proteins or small molecules. DARPINs are composed of a repeating structural motif called the ankyrin repeat, which is found in many proteins that bind to other molecules. The ankyrin repeat is a stable, modular protein domain that can be easily manipulated to create new binding specificities. DARPINs are designed using protein engineering techniques, including phage display and rational design, and can be tailored to have a range of desirable properties, such as high affinity, stability, and solubility. In yet some further embodiments, the DARPIN applicable in the present disclosure are T cell specific DATPINs. In some embodiments, T cell specific DARPins useful in the present disclosure include, but are not limited to DARPin CD8, DARPin CD4, DARPin CD3, DARPin MEDI3039 (designed to bind to the programmed death ligand 1 (PD-L1) protein), DARPin L-selectin (found on the surface of T cells), or any combinations thereof.

As shown by the following Examples, anti human CD4 and CD8 DARPINs transduction was compared and CD8 DARPIN showed higher transduction and specificity rates. CD8 is expressed on cytotoxic T cells, therefore, in some embodiments, CD8 DARPIN may be used. Furthermore, targeted vectors allowed for higher transduction rate compared with w.t AAV-DJ serotype. Moreover, the integration of DARPIN in the VP1 protein of the DJ serotype was not shown before, and a specific location was chosen, combined also with abolishment of heparin sulfate binding site to further increase specificity. Thus, in some embodiments, the DARPin used in the present disclosure as the targeting moiety may be the DARPin CD8. Still further, in some embodiments, the DARPin CD8 targets the viral vector to CD8+ T cells. In some embodiments the DARPin CD8 may comprise the amino acid sequence as denoted by SEQ ID NO: 38, or any derivatives and variants thereof. In yet some further embodiments, the DARPin CD8 used in the AAV vectors of the present disclosure may be the DARPin CD8 encoded by the nucleic acid sequence as denoted by SEQ ID NO: 31, or any variants and homologs thereof.

In some embodiments, the nucleic acid molecule of interest that encodes the product of interest, specifically, the therapeutic product, e.g., the CAR and/or TCR and/or the nucleic acid sequence encoding the at least one nuclease, or any coding cassettes and/or constructs thereof, may be provided in the same vector, and/or delivery vehicle, that may comprise at least one targeting moiety. In yet some further embodiments, the nucleic acid molecule of interest, e.g., the CAR and/or TCR and/or nuclease coding cassette and/or construct may be provided in separate vectors and/or delivery vehicles. According to such embodiments, the at least one of the nuclease vector/s and/or delivery vehicle/s and/or the nucleic acid molecule of interest, e.g., the CAR/TCR vector and/or delivery vehicle, can further comprise at least one targeting moiety promoting preferential T cell targeting in vivo. These moieties may be any affinity molecule specific for the target cell, for example, antibody derivatives, DARPin derivatives, aptamer/s, ligands of T cell receptors and/or receptors of T cell ligands.

As indicated above, in some embodiments, the vector and/or delivery vehicle used by the disclosed systems for delivering the nucleic acid molecule of interest and/or the at least one nuclease and/or any nucleic acid sequence encoding the same, may be an AAV vector. In some embodiments, the AAV vector may be an AAV vector expressing in its capsid, at least one T cell targeting moiety, specifically, DARPIN derivatives. In yet some further embodiments, the AAV vector used in the systems disclosed herein may comprise DARPin incorporated into the capsid and/or associated with the viral capsid. In yet some further embodiments, the AAV vector used herein may be a modified AAV vector having a capsid composed of DARPin that may replace the VP1 capsid protein or any fragments thereof.

In some embodiments, the DARPin used herein may replace the VP2 protein or any parts or fragments thereof. In some embodiments, the DARPin used herein may replace the VP3 protein or any parts or fragments thereof. In some embodiments, the AAV capsid proteins VP1, VP2 and VP3 may comprise the amino acid sequence as denoted by SEQ ID NO: 36, or any mutants, variants or derivatives thereof. In yet some further embodiments, the AAV capsid protein VP1 is encoded by the nucleic acid sequence as denoted by SEQ ID NO: 30, or any variants or mutants thereof. Still further, in some embodiments, the AAV capsid proteins VP2 and VP3 are encoded by the nucleic acid sequence as denoted by SEQ ID NO: 33, or any variants or mutants thereof. In yet some further embodiments, AAV VP2, VP3 mutant useful in the present disclosure may be encoded by a nucleic acid sequence comprising SEQ ID NO: 34, or any variants or mutants thereof.

In some embodiments, the AAV vector used in the present disclosure may comprise the VP1-DARPin CD8 encoded by the nucleic acid sequence as denoted by SEQ ID NO: 32, or any variants and homologs thereof. In some embodiments, the VP1-DARPin CD8 used in the present disclosure may comprise the amino acid sequence as denoted by SEQ ID NO: 37.

Still further, in some embodiments, the nucleic acid construct used for constructing the modified AAV-VP1-DARPin CD8 of the present disclosure may comprise the nucleic acid sequence as denoted by SEQ ID NO: 35, or any variants and homologs thereof.

Thus, the disclosed systems as well as the compositions and methods discussed herein after may comprise any of the AAV vectors, specifically VP1-DARPin modified AAV vector disclosed in the present disclosure.

In some embodiments the AAV-DARPin vector used herein may comprise the amino acid sequence as denoted by SEQ ID NO: 37, and any variants and derivatives thereof. In some embodiments, the at least one exogenous nucleic acid sequence of interest comprised as component (a) in the system of the present disclosure, may further comprise inducible suicide gene. Thus, in some embodiments, the integrated cassette (flanked with the homology arms) may further comprise an inducible suicide gene, thereby enabling the specific elimination of engineered T cells if adverse events take place. In some embodiments, the nucleic acid sequence of interest and the cassettes, systems and/or kits, compositions, methods and uses of the present disclosure, may further include sequence encoding at least one suicide gene product.

As used herein, the term “suicide gene” refers to a class of genes that produce proteins that promote the death of cells in which they expressed. Suicide genes that can be employed in the nucleic acid cassette provided by the systems and/or kits, compositions, methods and uses of the present disclosure include the caspases, CASP3, CASP8, CASP9, BAX, DFF40 and Fas. Further non-limiting examples of suicide genes include genes that encode a peptide or polypeptide that is cytotoxic either alone or in the presence of a cofactor, e.g. a toxin such as abrin, ricin A, Pseudomonas exotoxin, cholera toxin, diphtheria toxin, Herpes Simplex Thymidine Kinase (HSV-TK) and cytidine deaminase and genes that target a cell for ADCC or CDC-dependent death, e.g. CD20. It should be noted that in some embodiments a suicide gene may be a toxic gene. In some embodiments, a suicide gene may be added to the cassette which may be inducible i.e. which expression may be controlled at wish. In particular embodiments, an upstream caspase gene fused to an inducible dimerization domain (for example iCasp9) may be added to the cassette, as the default expression of iCasp9 is as an inactive monomeric caspase.

The nucleic acid sequence of interest and/or the nucleic acid sequence that encodes at least one nuclease, that may be comprised within the cassette provide by the present disclosure and by the systems and/or kits, compositions, methods and uses of the present disclosure, may encode a protein or polypeptide as described herein above (e.g., receptors or antibodies). The term “polypeptide” as used herein refers to amino acid residues, connected by peptide bonds. A polypeptide sequence is generally reported from the N-terminal end containing free amino group to the C-terminal end containing free carboxyl group and may include any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that contains portions that occur in nature separately from one another (i.e., from two or more different organisms, for example, human and non-human portions). In some embodiments, a polypeptide has an amino acid sequence that is engineered in that it is designed and/or produced through action of the hand of man. More specifically, “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

It should be appreciated that the present disclosure encompasses the use of any variant or derivative of the polypeptides of the invention and any polypeptides that are substantially identical or homologue to the polypeptides encoded by the nucleic acid sequence of the invention. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof. Proteins orthologs or homologues having a sequence homology or identity to the proteins of interest in accordance with the invention, specifically, receptors, chimeras and antibodies described herein, may share at least 50%, at least 60% and specifically 65%, 70%, 75%, 80%, 85%, 90%, 91%, 92%, 93%, 94%, 95%, 96%, 97%, 98%, 99%, or higher, specifically as compared to the entire sequence of the proteins of interest in accordance with the invention, for example, any of the proteins that comprise the amino acid sequence as denoted by SEQ ID NO. 4, SEQ ID NO: 7, and any derivatives and variants thereof. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO: 4, SEQ ID NO: 7, and any derivatives and variants thereof, specifically, the entire sequence as denoted by SEQ ID NO: 4, SEQ ID NO: 7, and any derivatives and variants thereof.

In some embodiments, derivatives refer to polypeptides, which differ from the polypeptides specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N‘ or C’ termini thereof. With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

Thus, in some embodiments, the invention encompasses targeting of any nucleic acid sequence of interest that encodes any of the specified polypeptides (e.g., receptors, chimeric receptors and antibodies), or any derivatives thereof, specifically a derivative that comprise at least 1, 2, 3, 4, 5, 6, 7, 8, 9, 10 or more conservative substitutions to the amino acid sequences as denoted by any one of SEQ ID NO: 4, SEQ ID NO: 7, and any derivatives and variants thereof. More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

Variants of the polypeptides of the present disclosure may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the various polypeptides of the present disclosure.

A further aspect of the present disclosure relates to a viral vector for in vivo targeted insertion of at least one exogenous nucleic acid sequence of interest into a target genomic locus of a cell of the T lineage in a mammalian subject. More specifically, the delivery vehicle may comprise at least one of: (a), at least one nucleic acid molecule comprising at least one exogenous nucleic acid sequence of interest. The sequence is flanked on at least one of the 5′ and 3′ thereof by at least one homology arm, for integration to the target locus by homologous recombination; and/or (b), at least one site specific nuclease, or at least one nucleic acid sequence encoding the nuclease.

In some embodiments, the viral vector may be an AAV vector, and/or AAV-like vector, and/or any AAV transducing particle and/or recombinant AAV vector. Specifically, any of the AAV vectors disclosed by the present disclosure.

In yet some further embodiments, the viral vector, specifically, AAV vector disclosed herein may further comprise at least one targeting moiety.

Still further, in some specific embodiments, the viral vector, specifically, AAV vector of the present disclosure may comprise at least one targeting moiety incorporated in the capsid thereof. In some optional embodiments, the at least one targeting moiety may replace at least one capsid protein of the viral vector, or at least part thereof.

In some embodiments, the viral vector of the present disclosure may be an AAV vector. In yet some further embodiments, the AAV vector may comprise at least one T cell targeting moiety. Still further, in some embodiments, the targeting moiety may be incorporated in the capsid of the disclosed AAV vector. Still further, in some embodiments, the targeting moiety may replace, at least partially, at least one of the capsid proteins of the AAV vector disclosed herein. In yet some further embodiments, the targeting moiety may replace, at least partially the VP1, and/or VP2, and/or VP3 capsid proteins of the AAV vector disclosed herein. In some particular embodiments, the AAV vector of the present disclosure may comprise DARPin molecule as the targeting moiety. Still further, in some embodiments, the AAV vector of the present disclosure may comprise DARPin molecules incorporated into the AAV capsid. In more specific embodiments, DARPin molecule may replace the capsid VP1 protein, or at least part thereof, of the AAV vector.

In some embodiments, the AAV Capsid engineering of the disclosed AAV vector, was made upon AAVDJ backbone by genetically fusing the anti-CD8 DARPIN into the GH2-GH3 surface loop of the VP1 capsid gene of AAV-DJ. In order to disrupt heparin sulfate binding, Arginines R587 and R590 of VP1 were mutated to Alanine residues in the plasmid encoding the VP1-DARPIN fusion. Still further, in some embodiments, the splice acceptor (SA) of the disclosed AAV vector may be inactivated to prevent incorporation of the DARPIN into the VP2 and VP3 capsid proteins. Expression of unmodified VP2 and VP3 is provided by a second plasmid, in which the start codon (Met) of VP1 was inactivated to prevent incorporation of unmodified VP1 in the capsid. In some embodiments, the AAV vector used in the present disclosure may comprise the VP1-DARPin CD8 encoded by the nucleic acid sequence as denoted by SEQ ID NO: 32, or any variants and homologs thereof. In some embodiments, the VP1-DARPin CD8 used in the present disclosure may comprise the amino acid sequence as denoted by SEQ ID NO: 37, or any derivatives thereof.

Still further, in some embodiments, the nucleic acid construct used for constructing the modified AAV-VP1-DARPin CD8 of the present disclosure may comprise the nucleic acid sequence as denoted by SEQ ID NO: 35, or any variants and homologs thereof.

In some embodiments, the viral vector, specifically, AAV vector of the present disclosure may comprise any of the systems of the present disclosure. In some embodiments, the disclosed viral AAV vectors may comprise any of the nucleic acid cassettes disclosed by the present disclosure, as described in more detail in connection with other aspects of the present disclosure.

In some embodiments, the present disclosure provides the disclosed AAV-VP1-DARPIN vector that comprise at least one nucleic acid molecule comprising the nucleic acid sequence encoding the nucleic acid sequence of interest, and the nucleic acid sequence encoding the at least one nuclease. In some embodiments, the modified AAV-VP1-DARPin CD8 disclosed herein may comprise the amino acid sequence as denoted by SEQ ID NO: 37, or any derivatives thereof. Still further, in some embodiments, the modified AAV-VP1-DARPin CD8 of the present disclosure may be constructed using a construct that comprise the nucleic acid sequence as denoted by SEQ ID NO: 35, or any variants and homologs thereof. The present disclosure therefore provides the AAV-VP1-DARPIN vectors that comprise each of the following nucleic acid molecules.

More specifically, in some embodiments, the AAV-VP1-DARPIN vector may comprise a nucleic acid molecule, and/or cassette that comprise from the 5′ end thereof, a left homology arm followed by a splice acceptor site (SA), furin-2A, the nucleic acid sequence of interest that in some embodiments encode at least one therapeutic and/or modulatory molecule, followed by a polyA sequence and flanked by a right homology arm, the cassette further comprises an exogenous promoter, an NLS coding sequence and a nucleic acid sequence encoding at least one nuclease followed by a polyA sequence. A schematic presentation of such cassette is disclosed by FIG. 1A(i), also referred to herein as the first strategy. This strategy holds an advantage of expressing the CAR under the TRAC endogenous promoter and regulation, therefore preventing T cell exhaustion and reduced potency. The dependency of CAR expression under the TRAC locus also adds another level of specificity; therefore, in some embodiments, viral vectors comprising these disclosed nucleic acid molecules and/or cassettes, are used. In some specific embodiments, the nucleic acid sequence of interest flanked by both homology arms, encodes at least one therapeutic and/or modulatory molecule. In some embodiments, the nucleic acid sequence of interest encodes at least one therapeutic protein. In more specific embodiments, the therapeutic protein may be a receptor molecule. Still further in some embodiments, such receptor molecule may be a CAR molecule and/or a TCR molecule. In some specific embodiments, the nucleic acid sequence of interest encodes at least one CAR molecule, for example, a CAR molecule directed at the CD19 antigen. Still further, in some embodiments the hCD19 CAR molecule may comprise the amino acid sequence as denoted by SEQ ID NO: 4. In yet some further embodiments, the CAR molecule of the cassettes disclosed in the present disclosure may comprise at least one nucleic acid sequence encoding the hCD19 CAR molecule. In some embodiments, such sequence may comprise the nucleic acid sequence as denoted by SEQ ID NO: 5, or any variants and homologs thereof. Still further, in some embodiments, the nuclease encoded by the cassette of the present disclosure may be the ARCUS nuclease. In some specific embodiments, the ARCUS nuclease may specifically recognize a target locus within the TRAC gene. Still further, in some embodiments the ARCUS disclosed by the present disclosure may comprise the amino acid sequence as denoted by SEQ ID NO: 7, or any derivatives and variants thereof. Still further, in some embodiments, the ARCUS may be encoded by the nucleic acid sequence as denoted by SEQ ID NO: 6, or any homologs or variants thereof. Thus, in some particular embodiments, the cassette of the present disclosure may further comprise the nucleic acid sequence as denoted by SEQ ID NO: 6. Still further, in some embodiments, the ARCUS provided in the disclosed cassette is under the exogeneous JeT promoter. In more specific embodiments, the cassette of the present disclosure may comprise the nucleic acid sequence as denoted by SEQ ID NO: 19, or any variants and derivatives thereof.

In yet some alternative embodiments, the AAV-VP1-DARPIN vector disclosed herein may comprise a nucleic acid molecule, and/or cassette that may comprise from the 5′ end thereof, a left homology arm followed by a splice acceptor site (SA), followed by a polyA sequence, a JeT promoter and at least one nucleic acid sequence encoding the therapeutic molecule of interest (e.g., CAR molecule, specifically, the hCD19 CAR molecule, followed by a Furin-2A sequence, a splice donor site (SD), and a right homology arm, followed by a second SA, NLS sequence and a sequence encoding the nuclease (e.g., ARCUS) followed by a poly A sequence. A schematic presentation of such cassette is disclosed by FIG. 1A(ii), also referred to herein as the second strategy.

In yet some other alternative embodiments, the AAV-VP1-DARPIN vector disclosed herein may comprise a nucleic acid molecule, and/or cassette that may comprise from the 5′ end thereof, an exogenous promoter sequence (e.g. JeT promoter), followed by NLS and a sequence encoding at least one nuclease (e.g., the ARCUS), followed by a Furin-2A sequence, a left homology arm followed by a splice acceptor site (SA), followed by at least one nucleic acid sequence encoding the therapeutic molecule of interest (e.g., CAR molecule, specifically, the hCD19 CAR molecule, followed a poly A sequence and a right homology arm sequence. A schematic presentation of such cassette is disclosed by FIG. 1A(iii), also referred to herein as the third strategy.

In some specific embodiments of the AAV-VP1-DARPIN vector disclosed herein may comprise a nucleic acid molecule, and/or cassette that may comprise at least one nucleic acid sequence of interest that encodes at least one CAR molecule, for example, a CAR molecule directed at the CD19 antigen. Still further, in some embodiments the hCD19 CAR molecule may comprise the amino acid sequence as denoted by SEQ ID NO: 4. In yet some further embodiments, the CAR molecule of the cassettes disclosed in the present disclosure may comprise at least one nucleic acid sequence encoding the hCD19 CAR molecule. In some embodiments, such sequence may comprise the nucleic acid sequence as denoted by SEQ ID NO: 5, or any variants and homologs thereof. Still further, in some embodiments, the nuclease encoded by the cassette of the present disclosure may be the ARCUS nuclease. In some specific embodiments, the ARCUS nuclease may specifically recognize a target locus within the TRAC gene. Still further, in some embodiments the ARCUS disclosed by the present disclosure may comprise the amino acid sequence as denoted by SEQ ID NO: 7, or any derivatives and variants thereof. Still further, in some embodiments, the ARCUS may be encoded by the nucleic acid sequence as denoted by SEQ ID NO: 6, or any homologs or variants thereof. Thus, in some particular embodiments, the cassette of the present disclosure may further comprise the nucleic acid sequence as denoted by SEQ ID NO: 6, or any homologs or variants thereof.

It should be appreciated that the second and third strategies as used herein, provide episomal CAR expression which could mitigate tumor progression, allowing more T cells to express the CAR at earlier time points. These two strategies include sophisticated designs, in which one transcript allows the translation of both the CAR/TCR and the ARCUS enzyme from the episomal AAV, but only the CAR/TCR is further expressed upon integration (because the ARCUS enzyme is coded outside of the homology arms). In each version a carefully planned combination of several elements was used by the present inventors to save space between the ITRs (splice acceptor, splice donor, 2A peptide, Furin cleavage site, GSG linkers, etc.). Of note, in the constructs of strategy 2, the CAR gene is followed by a 2A peptide, positioned to allow a continuous reading frame with the downstream TRAC exons, in order to prevent nonsense mediated decay (NMD). In the construct of strategy 3, the CAR/TCR gene is preceded by a 2A peptide, to allow a continuous reading frame with the preceding VJ exon, to allow endogenous regulation by the V promoter upon integration. When using a 2A peptide, the first protein is sometimes expressed at a higher rate than the second protein. Therefore, the two different strategies were created and used by the present disclosure.

In some further aspects thereof, the present disclosure further comprises matrix, nano- or micro-particle and/or composition comprising the system of the invention. In some embodiments, such composition may further comprise at least one pharmaceutically acceptable carrier/s, excipient/s, auxiliaries, and/or diluent/s.

A further aspect of the present disclosure relates to a method for in vivo targeted insertion of at least one exogenous nucleic acid sequence of interest into a target genomic locus of a cell of the T lineage, in a mammalian subject. The disclosed methods may comprise the step of administering to the subject an effective amount of:

(a), at least one nucleic acid molecule comprising the at least one exogenous nucleic acid sequence of interest, and/or at least one cassette/s and/or construct/s thereof. The sequence is flanked on at least one of the 5′ and 3′ thereof by at least one homology arm, for integration to the target locus by homologous recombination. It should be noted that the at least one nucleic acid molecule and/or at least one cassette/s and/or construct/s is comprised within at least one AAV vector and/or AAV-like vector.

The subject is further administered with (b), at least one site specific nuclease, or at least one nucleic acid sequence encoding the nuclease or at least one cassette, and/or construct, and/or vector and/or delivery vehicle comprising the at least one nuclease or said nucleic acid sequence encoding the nuclease.

Alternatively (c), the subject is administered with any cassette, and/or construct, and/or vector and/or system, and/or delivery vehicle, matrix, nano- or micro-particle and/or composition comprising (a) and/or (b).

In some embodiments, the target locus targeted by the in vivo targeting method of the present disclosure is at least one of TCR α chain locus, TCRβ chain locus, TCRγ chain locus and the TCRS chain locus.

In some embodiments, the target locus targeted by the in vivo targeting method of the present disclosure is at least one of the TRAC, and the TRBC loci.

In some embodiments, the target locus targeted by the in vivo targeting method of the present disclosure, may be the TRAC locus. It should be noted that the at least one homology arm enables the integration of the at least one exogenous nucleic acid sequence of interest into the TRAC locus.

In some embodiments, the at least one site specific nuclease used by the in vivo targeting method of the present disclosure, may be at least one of: at least one homing endonuclease, at least one ZFN, at least one TALEN, at least one CRISPR/Cas protein, and at least one Mega-TAL.

In more specific embodiments, the homing nuclease used by the in vivo targeting method of the present disclosure, may be at least one member of the LAGLIDADG family of homing endonucleases.

In some embodiments, the at least one member of the LAGLIDADG family of homing endonucleases used by the in vivo targeting method of the present disclosure may be endonuclease I-CreI, or an engineered derivative thereof.

Still further, in some embodiments, the engineered derivative of endonuclease I-CreI used by the in vivo targeting method of the present disclosure, may be the ARCUS endonuclease that specifically targets the TRAC locus.

In yet some further embodiments, the exogenous nucleic acid sequence of interest inserted by the in vivo targeting method of the present disclosure, may encode any therapeutic molecule. In some embodiments, the therapeutic molecule may be at least one therapeutic protein and/or a therapeutic or modulatory nucleic acid molecule (e.g., miRNA, shRNA, etc.). In some embodiments the therapeutic protein may be at least one receptor molecule. Thus, in some embodiments, the nucleic acid molecule of interest may comprise at least one nucleic acid sequence that encodes at least one of: at least one CAR molecule and/or at least one exogenous and/or engineered TCR molecule.

Still further in some embodiments, the nucleic acid sequence of interest encodes at least one CAR molecule.

In more particular embodiments, the in vivo targeting method of the present disclosure provides the in vivo targeted insertion of nucleic acid sequence encoding anti human CD19 CAR.

In some embodiments, the nucleic acid sequence of interest encodes at least one exogenous TCR. In yet some further embodiments, the targeted insertion of the exogenous TCR and/or CAR into the TRAC/TRBC loci, disrupts or at least reduces in about 5% to about 100% the expression of the endogenous TCR by the T cells. Thus, in some embodiments, cells of the T lineage that underwent an in vivo targeted insertion of at least one nucleic acid sequence of interest into the TRAC locus, do not express an endogenous TCR, or display reduced or decreased expression of endogenous TCR. In yet some alternative embodiments, the CAR or TCR encoding nucleic acid sequences used in the disclosed methods may be preceded by at least one splice acceptor followed by a 2A peptide or an IRES sequence, thereby allowing the CAR or TCR to be transcribed together with the endogenous variable domains of the TCR chains. The CAR or TCR is separated from the variable domains of the endogenous TCR chains upon translation due to the polycistronic coding facilitated by the 2A peptide or IRES sequences.

Still further, in some embodiments, the at least one nucleic acid molecule comprising the at least one exogenous nucleic acid sequence of interest (a), and the nucleic acid sequence encoding at least one site specific nuclease (b), administered by the in vivo targeting methods of the present disclosure, are comprised within the same cassette/s and/or construct/s, and/or vector/s. It should be noted that the vector is at least one AAV vector and/or AAV-like vector.

The disclosed methods therefore involve administering to a subject in need, at least one nucleic acid molecule comprising (a), at least one exogenous nucleic acid sequence of interest, flanked on at least one of the 5′ and 3′ thereof by at least one homology arm, for integration to a target locus by homologous recombination; and (b), at least one site specific nuclease, or at least one nucleic acid sequence encoding the nuclease, or any cassette/s and/or construct/s, and/or vector/s, and/or systems and/or delivery vehicle/s comprising (a) and (b).

In some alternative embodiments, the at least one nucleic acid molecule comprising the at least one exogenous nucleic acid sequence of interest (a), and the nucleic acid sequence encoding at least one site specific nuclease (b), are provided in separate cassette/s and/or construct/s, and/or vector/s and/or delivery vehicle/s. Therefore, in some embodiments, components (a) and (b) may be administered separately by the in vivo targeting methods of the present disclosure. In some embodiments, the administration of (a), the at least one exogenous nucleic acid sequence of interest, or any cassette/s and/or construct/s, and/or vector/s and/or delivery vehicle/s thereof, or (b), the nucleic acid sequence encoding at least one site specific nuclease or any cassette/s and/or construct/s, and/or vector/s and/or delivery vehicle/s thereof, may be administered either together or separately at either order. It should be noted that the at least one nucleic acid molecule and/or at least one cassette/s and/or construct/s thereof, is comprised within at least one AAV vector and/or AAV-like vector.

In some embodiments, the nuclease may be provided in separate vector/s and/or delivery vehicles, it can be in some embodiments provided as mRNA or a modified mRNA, coding for the nuclease. Alternatively, the nuclease may be provided in the disclosed system as a protein. Such protein may be provided in a separate vector or delivery vehicle.

In some embodiments, the cassette/s and/or construct/s, and/or vector/s and/or delivery vehicle/s used by the in vivo targeting method of the present disclosure, may further comprise at least one genetic element. In some embodiments, the genetic element may be at least one of: at least one IRES, at least one 2A peptide coding sequence, at least one promoter or any functional fragments thereof, at least one SD, at least one SA, at least one degron, at least one 3 frame stop, at least one protein stabilizing sequence, at least one signal peptide, at least one stop codon, at least one polyadenylation site, at least one transcription enhancer, at least one switch region, at least one mRNA stabilizing sequence and at least one protein stabilizing sequence.

In some embodiments, the nucleic acid sequence of interest encodes in some embodiments, at least one therapeutic and/or modulatory molecule. In some embodiments, the nucleic acid sequence of interest encodes at least one therapeutic protein. In more specific embodiments, the therapeutic protein may be a receptor molecule. Still further in some embodiments, such receptor molecule may be a CAR molecule and/or a TCR molecule. Thus, in some embodiments, the CAR and/or the TCR coding nucleic acid sequences (or sequence coding for any therapeutic molecule as discussed herein), may be integrated with a promoter, which may be T cell specific, and may further be active only upon genomic integration of the cassette that comprise the nucleic acid sequence of interest (e.g., CAR and/or TCR encoding sequences), as when it is placed in proximity to the necessary enhancers of the TRAC and/or TRBC loci.

In yet some further embodiments, the cassette used herein may comprise exogenous promoter. In yet some further embodiments, the cassette used by the disclosed methods may utilize the endogenous TCR promoter.

In yet some further embodiments, the vector applicable in the in vivo targeting method of the present disclosure is any one of a viral vector, a non-viral vector and a naked DNA vector.

In more specific embodiments, the vector useful in the in vivo targeting method of the present disclosure may be a viral vector. In some embodiments, the viral vector may be any one of AAV, AAV-like, rAAV, ssAAV, scAAV, SV40 vector, Adeno virus vector, helper-dependent Adeno viral vector, retroviral vector and/or lentiviral vector.

In some embodiments, the vector and/or delivery vehicle used by the disclosed methods for delivering the nucleic acid molecule of interest and/or the at least one nuclease and/or any nucleic acid sequence encoding the same, may be an AAV vector. The AAV vector may be either a native vector or a modified, and/or genetically engineered AAV vector. In certain embodiments, the disclosed in vivo methods may use a non-viral vector, more specifically, such vector may be any one of plasmid, minicircle and linear DNA.

In some embodiments, such vector is a naked DNA vector. Specifically, the vector is any one of plasmid, minicircle and linear DNA.

In some embodiments, the at least one vector and/or delivery vehicle used by the in vivo targeting method of the present disclosure further comprises at least one T cell targeting moiety. These moieties may be in some embodiments, any affinity molecule specific for the target cell, for example, antibody derivatives, DARPin derivatives, aptamer/s, ligands of T cell receptors and/or receptors of T cell ligands.

As noted above, in some embodiments, the delivery vehicle used by the disclosed methods may be an AAV vector. In some embodiments, the AAV vector may be an AAV vector expressing in its capsid at least one T cell targeting moiety, specifically, DARPIN derivatives. In yet some further embodiments, the AAV vector used in the methods disclosed herein may comprise DARPin incorporated in the capsid. In yet some further embodiments, the AAV vector used herein may be a modified AAV vector having a capsid composed of DARPin that may replace the VP1 capsid protein or any fragments thereof.

In some embodiments, the modified AAV-VP1-DARPin CD8 used by the methods of the present disclosure may comprise the amino acid sequence as denoted by SEQ ID NO: 37, or any derivatives thereof. Still further, in some embodiments, the modified AAV-VP1-DARPin CD8 used by the methods of the present disclosure may be constructed using a construct that comprise the nucleic acid sequence as denoted by SEQ ID NO: 35, or any variants and homologs thereof.

In some embodiments, when the nucleic acid molecule of interest (e.g., that encodes the CAR and/or TCR) and the nuclease coding vector, are provided in separate vectors, at least one of the nuclease vector and the vector of the nucleic acid molecule of interest (e.g., that encodes the CAR and/or TCR), can further comprise at least one targeting moiety promoting preferential T cell targeting in vivo. As discussed above, these moieties may be antibody derivatives, DARPIN derivatives, ligands of T cell receptors or receptors of T cell ligands.

In yet some further embodiments, the at least one exogenous nucleic acid sequence of interest used by the in vivo targeting methods of the present disclosure, may further comprise an inducible suicide gene.

Still further, in some embodiments, the in vivo targeting methods of the present disclosure may be performed in a subject in need. In some embodiments, such a subject may be a mammalian subject suffering from an immune-related disorder.

As indicated herein, the methods of the present disclosure involve the administration of the systems, delivery vehicles, kits, and/or constructs provided by the present disclosure to a subject in need thereof, for enabling the in vivo targeted insertion of the nucleic acid sequence of interest into the specific target locus in the target cells in the subject. Therefore, the systems, delivery vehicles and compositions of the invention may be adapted in some embodiments of the present disclosure to any appropriate systemic and/or local administration mode, such that the nucleic acid sequence of interest can be delivered to the target cells in the administered subject.

Still further, in some embodiments for in vivo targeted insertion of the nucleic acid sequence of interest, the nucleic acid cassette or any vector and/or delivery vehicle comprising the same used by the systems, compositions, and/or method of the preset disclosure, may be administered by at least one of systemic injection, intrathymic injection, bone marrow injection splenic injection and injection to lymph nodes.

More specifically, in some embodiments where engineered T cells are desired, the method of the present disclosure intrathymic injection. Intrathymic injection is a procedure used in several T cell-associated immunological contexts to deliver cells or other substances directly into the thymus. In the context of the present invention, the nucleic acid cassette of the invention or any vector or composition thereof may be injected into the thymus, thereby specifically targeting differentiating T cells. In some embodiments, the system/kit, compositions and nucleic acid cassette or the vectors may be injected via intrathymic injection, in such case the target cells may be thymocytes, specifically, thymocytes of the DN1, DN2, DN3, DN4, DP and SP subsets).

In case of bone marrow injection, the target cells may be HSPCs and in case of systemic injection, the target cells may be mobilized HSPCs (where the patient is subjected to a preceding treatment of immobilization). It should be noted that both the bone marrow injection as well as systemic injection (e.g., intravenous IV), may be specifically suitable for targeting T cells by the nucleic acid cassette of the invention. It is to be understood that other localized injections may be also suitable, for example intra-lymph node injection or intra-spleen injection and may be used to deliver a vector to the lymph node and the spleen, respectively. More specifically, in some embodiments, the subcutaneous route (SC) may be generally considered to be most appropriate for targeting to the lymph nodes. Conventional SC formulations are such as Aqueous solutions, Oily solutions, Suspensions and Simple emulsions. Specific technologies associated with SC delivery are via Modified release SC formulations such as Biodegradable in situ implants, Biodegradable microspheres, osmotically controlled implants, Liposomes, Lipid nanoparticles. Relevant commercially available products may include but are not limited to Alzamer® Depot™, DUROS®, Stealthl (ALZA Corporation), Atrigel® (Atrix Laboratories), SABER® (Durect Corp), ProLease (Alkermes Inc), DepoFoam® (SkyePharma Inc), SupraVail™ (Phares Drug Delivery AG).

A further aspect of the present disclosure relates to a method for treating, preventing, ameliorating, inhibiting or delaying the onset of an immune-related disorder in a mammalian subject. Specifically, the method comprising the step of administering to the subject a therapeutically effective amount of:

(a), at least one nucleic acid molecule comprising at least one exogenous nucleic acid sequence of interest and/or at least one cassette/s and/or construct/s thereof. The sequence is flanked on at least one of the 5′ and 3′ thereof by at least one homology arm, for integration by homologous recombination to at least one target locus of a cell of the T lineage in the subject. The subject may be administered in some embodiments with at least one cassette/s and/or construct/s, and/or vector/s comprising the at least one nucleic acid molecule. It should be noted that the at least one nucleic acid molecule and/or at least one cassette/s and/or construct/s is comprised within at least one AAV vector and/or AAV-like vector; and (b), at least one site specific nuclease, or at least one nucleic acid sequence encoding the nuclease or any cassette, vector or vehicle comprising the at least one nuclease or the nucleic acid sequence encoding the nuclease. Alternatively, the subject may be administered with (c), any system, delivery vehicle, matrix, nano- or micro-particle and/or composition comprising (a) and/or (b).

In some embodiments, the target locus targeted by the therapeutic methods of the present disclosure may be at least one of TCR α chain locus, TCRβ chain locus, TCRγ chain locus and/or the TCRδ chain locus.

In yet some further embodiments, the target locus targeted by the therapeutic methods of the present disclosure may be at least one of the TRAC, and/or the TRBC loci.

In some embodiments, the target locus may be the TRAC locus. Thus, in some embodiments, the at least one homology arm enables the integration of the at least one exogenous nucleic acid sequence of interest into the TRAC locus.

In some embodiments, the at least one site specific nuclease used by the therapeutic methods of the present disclosure, may be at least one of: at least one homing endonuclease, at least one ZFN, at least one TALEN, at least one CRISPR/Cas protein, and/or at least one Mega-TAL.

In more specific embodiments, the homing nuclease used by the therapeutic methods of the present disclosure may be at least one member of the LAGLIDADG family of homing endonucleases. In some embodiments, at least one member of the LAGLIDADG family of homing endonucleases may be endonuclease I-CreI, or an engineered derivative thereof.

In still further specific embodiments, the engineered derivative of endonuclease I-CreI used by the therapeutic methods of the present disclosure, may be the ARCUS endonuclease that targets the TRAC locus.

In some further embodiments, the exogenous nucleic acid sequence of interest administered to the subject may encode at least one therapeutic and/or modulatory molecule. In some embodiments, the nucleic acid sequence of interest encodes at least one therapeutic protein. In more specific embodiments, the therapeutic protein may be a receptor molecule. Still further in some embodiments, such receptor molecule may be a CAR molecule and/or a TCR molecule.

In some embodiments, the nucleic acid sequence of interest administered to the subject may encode at least one CAR molecule. In some particular embodiments, the CAR molecule encoded by the nucleic acid sequence used by the therapeutic methods of the present disclosure may be the anti CD19 CAR, as disclosed herein.

It should be noted that the CAR molecule or any exogenous and/or engineered TCR encoded by the nucleic acid molecule of interest used by the disclosed methods may be directed against any antigen. In some embodiments, the antigen may be any antigen associated with any immune-related disorder. For example, any antigen associated with cancer. Thus, in some embodiments such TCR molecules and/or CAR molecules may be directed against any TAA, specifically, any of the TAAs disclosed in the present disclosure. Alternatively, the TCR molecules and/or CAR molecules may be directed against any antigen associated with a pathogenic entity (e.g., viral, bacterial and the like). In some embodiments, the nucleic acid sequence of interest administered by the disclosed methods may encode at least one exogenous and/or engineered TCR. In yet some further embodiments, the targeted insertion of the exogenous TCR molecules and/or CAR molecules into the TRAC and/or TRBC loci, disrupts or at least reduces (e.g., by at least about 5% to about 100%) the expression of the endogenous TCR by the in vivo engineered T cells. In yet some alternative embodiments, the nucleic acid sequences of interest that encode in some embodiments a desired therapeutic molecule (e.g., CAR or TCR), may be preceded by a splice acceptor followed by a 2A peptide or an IRES sequence, thereby allowing the CAR or TCR to be transcribed together with the endogenous variable domains of the TCR chains. The CAR or TCR is separated from the variable domains of the endogenous TCR chains upon translation due to the polycistronic coding facilitated by the 2A peptide or IRES sequences.

In some embodiments, the disclosed methods comprise the step of administering at least one nucleic acid cassette, or any delivery vehicle or vector thereof. In some embodiments, a cassette used by the disclosed method may comprise from the 5′ end thereof, a left homology arm followed by a splice acceptor site (SA), furin-2A, the nucleic acid sequence of interest that in some embodiments encode at least one therapeutic and/or modulatory molecule (e.g., TCR molecules and/or CAR molecules), followed by a polyA sequence and flanked by a right homology arm, the cassette further comprises an exogenous promoter, an NLS coding sequence and a nucleic acid sequence encoding at least one nuclease followed by a polyA sequence. A schematic presentation of such cassette is disclosed by FIG. 1A(i), also denoted herein as the first strategy.

In some specific embodiments, the nucleic acid sequence of interest flanked by both homology arms, encodes at least one therapeutic and/or modulatory molecule. In some embodiments, the nucleic acid sequence of interest encodes at least one therapeutic protein. In more specific embodiments, the therapeutic protein may be a receptor molecule. Still further in some embodiments, such receptor molecule may be a CAR molecule and/or a TCR molecule. In some specific embodiments, the nucleic acid sequence of interest encodes at least one CAR molecule, for example, a CAR molecule directed at the CD19 antigen. Still further, in some embodiments the hCD19 CAR molecule may comprise the amino acid sequence as denoted by SEQ ID NO: 4. In yet some further embodiments, the CAR molecule of the cassettes disclosed in the present disclosure may comprise at least one nucleic acid sequence encoding the hCD19 CAR molecule. In some embodiments, such sequence may comprise the nucleic acid sequence as denoted by SEQ ID NO: 5, or any variants and homologs thereof. Still further, in some embodiments, the nuclease encoded by the cassette of the present disclosure may be the ARCUS nuclease. In some specific embodiments, the ARCUS nuclease may specifically recognize a target locus within the TRAC gene. Still further, in some embodiments the ARCUS disclosed by the present disclosure may comprise the amino acid sequence as denoted by SEQ ID NO: 7, or any derivatives and variants thereof. Still further, in some embodiments, the ARCUS may be encoded by the nucleic acid sequence as denoted by SEQ ID NO: 6, or any homologs or variants thereof. Thus, in some particular embodiments, the cassette of the present disclosure may further comprise the nucleic acid sequence as denoted by SEQ ID NO: 6. Still further, in some embodiments, the ARCUS provided in the disclosed cassette is under the exogeneous JeT promoter. In more specific embodiments, the cassette of the present disclosure may comprise the nucleic acid sequence as denoted by SEQ ID NO: 19, or any variants and derivatives thereof.

In yet some alternative embodiments, the nucleic acid cassette used by the methods of the present disclosure may comprise from the 5′ end thereof, a left homology arm followed by a splice acceptor site (SA), followed by a polyA sequence, a JeT promoter and at least one nucleic acid sequence encoding the therapeutic molecule of interest (e.g., CAR molecule, specifically, the hCD19 CAR molecule, followed by a Furin-2A sequence, a splice donor site (SD), and a right homology arm, followed by a second SA, NLS sequence and a sequence encoding the nuclease (e.g., ARCUS) followed by a poly A sequence. A schematic presentation of such cassette is disclosed by FIG. 1A(ii), also denoted herein as the second strategy.

In yet some other alternative embodiments, the nucleic acid cassette used by the methods of the present disclosure may comprise from the 5′ end thereof, an exogenous promoter sequence (e.g. JeT promoter), followed by NLS and a sequence encoding at least one nuclease (e.g., the ARCUS), followed by a Furin-2A sequence, a left homology arm followed by a splice acceptor site (SA), followed by at least one nucleic acid sequence encoding the therapeutic molecule of interest (e.g., CAR molecule, specifically, the hCD19 CAR molecule, followed a poly A sequence and a right homology arm sequence. A schematic presentation of such cassette is disclosed by FIG. 1A(iii), also denoted herein as the third strategy.

In some specific embodiments of the cassettes used by the methods disclosed herein, the nucleic acid sequence of interest encodes at least one CAR molecule, for example, a CAR molecule directed at the CD19 antigen. Still further, in some embodiments the hCD19 CAR molecule may comprise the amino acid sequence as denoted by SEQ ID NO: 4. In yet some further embodiments, the CAR molecule of the cassettes disclosed in the present disclosure may comprise at least one nucleic acid sequence encoding the hCD19 CAR molecule. In some embodiments, such sequence may comprise the nucleic acid sequence as denoted by SEQ ID NO: 5, or any variants and homologs thereof. Still further, in some embodiments, the nuclease encoded by the cassette of the present disclosure may be the ARCUS nuclease. In some specific embodiments, the ARCUS nuclease may specifically recognize a target locus within the TRAC gene. Still further, in some embodiments the ARCUS disclosed by the present disclosure may comprise the amino acid sequence as denoted by SEQ ID NO: 7, or any derivatives and variants thereof. Still further, in some embodiments, the ARCUS may be encoded by the nucleic acid sequence as denoted by SEQ ID NO: 6, or any homologs or variants thereof. Thus, in some particular embodiments, the cassette of the present disclosure may further comprise the nucleic acid sequence as denoted by SEQ ID NO: 6, or any homologs or variants thereof.

In some embodiments, the at least one nucleic acid molecule comprising the at least one exogenous nucleic acid sequence of interest (denoted herein as (a)), and the nucleic acid sequence encoding at least one site specific nuclease (denoted herein as (b)) used by the therapeutic methods of the present disclosure, may be comprised within, and thus provided to the subject with the same cassette, construct, vector and/or delivery vehicle. According to such embodiments, the subject may be administered with a single delivery vehicle or vector and/or construct and/or composition that comprise both elements (a) and (b). It should be noted that the vector is at least one AAV vector and/or AAV-like vector.

In yet some alternative embodiments, the at least one nucleic acid molecule comprising the at least one exogenous nucleic acid sequence of interest (denoted herein as (a)), and the nucleic acid sequence encoding at least one site specific nuclease (denoted herein as (b)), used by the therapeutic methods of the present disclosure may be provided in separate cassette/s and/or construct/s, and/or vector/s and/or delivery vehicle/s. Thus, the subject may be administered with at least two delivery vehicles or vector/s and/or construct/s and/or composition/s, each comprise both one of (a) or (b). It should be noted that the at least one nucleic acid molecule and/or at least one cassette/s and/or construct/s thereof, is comprised within at least one AAV vector and/or AAV-like vector.

In some embodiments, the nuclease used by the therapeutic methods of the present disclosure may be provided in a separate vector. In some embodiments, the nuclease may be provided as mRNA or a modified mRNA, coding for the nuclease. Alternatively, the nuclease may be provided in the disclosed system as a protein. Such protein may be provided in a separate vector or delivery vehicle.

In some embodiments, the cassette/s and/or construct/s, and/or vector/s and/or delivery vehicle/s used by the therapeutic methods disclosed herein, may further comprise at least one genetic element. In some embodiments, the genetic element applicable herein may be at least one of: IRES, a 2A peptide coding sequence, a promoter or any functional fragments thereof, a SD, a SA, a degron, a 3 frame stop, a protein stabilizing sequence, a signal peptide, a stop codon, a polyadenylation site, a transcription enhancer, a switch region, an mRNA stabilizing sequence and a protein stabilizing sequence.

In some embodiments, the nucleic acid sequence of interest that encodes in some embodiments, at least one therapeutic and/or modulatory molecule, for example, at least one therapeutic protein, specifically, a receptor molecule such as CAR molecule and/or a TCR molecule, may be integrated with a promoter. In some embodiments, the promoter may be a T cell specific promoter and may further be active only upon genomic integration of the nucleic acid sequence of interest cassette (e.g., the CAR or TCR cassette). Specifically, in some embodiments, the activation of such T cell specific promoter may be facilitated or be possible when the nucleic acid cassette is placed in proximity to the necessary enhancers of the TRAC and/or TRBC loci.

In yet some further embodiments, the vectors and/or delivery vehicle comprising the nucleic acid molecules disclosed herein used by the therapeutic methods of the present disclosure, may be any one of a viral vector, a non-viral vector and a naked DNA vector.

In certain embodiments, the vector used herein may be a viral vector. In some embodiments, the viral vector may be any one of AAV vector and/or AAV-like vector, rAAV, ssAAV, scAAV, SV40 vector, Adeno virus vector, helper-dependent Adeno viral vector, retroviral vector and lentiviral vector. In some embodiments, the vector and/or delivery vehicle used by the disclosed methods for delivering the nucleic acid molecule of interest and/or the at least one nuclease and/or any nucleic acid sequence encoding the same, may be an AAV vector. In yet some further embodiments, the AAV vector may be either a native AAV vector or a genetically modified AAV vector.

In some alternative embodiments, the vector used by the disclosed therapeutic methods, is a non-viral vector, the vector may be any one of plasmid, minicircle and linear DNA.

In yet some further embodiments, the vector used by the disclosed therapeutic methods may be a naked DNA vector, in some embodiments the vector may be any one of plasmid, minicircle and linear DNA.

In yet some further embodiments, the vector used in the disclosed therapeutic methods may further comprise at least one targeting moiety. In some embodiments, at least one T cell targeting moiety.

In some embodiments, the nucleic acid molecule of interest (e.g., that encodes a therapeutic molecule, for example CAR/TCR, also indicated herein as component (a)) and nuclease coding vector (also indicated herein as component (b)), may be provided either in a single delivery vehicle or vector (e.g., a viral vector), or in at least two separate delivery vehicles (e.g. viral vectors). In some embodiments, when both components are provided in a single delivery vehicle or vector, such vector may further comprise at least one targeting moiety. In yet some further embodiments, when the two components are provided in separate vectors and/or deliver vehicles, at least one of the nuclease vectors, and/or the nucleic acid molecule of interest (e.g., CAR/TCR) vector, can further comprise at least one targeting moiety. In some embodiments, such targeting moiety may promote preferential T cell targeting in vivo. These moieties may be any affinity molecule, for example, antibody derivatives, DARPIN derivatives, ligands of T cell receptors, aptamers, or receptors of T cell ligands.

As indicated above, in some embodiments, at least one of the vectors and/or delivery vehicles used by the disclosed methods for delivering the nucleic acid molecule of interest and/or the at least one nuclease and/or any nucleic acid sequence encoding the same, may be an AAV vector. In some embodiments, the AAV vector may be an AAV vector expressing in its capsid, at least one T cell targeting moiety, specifically, DARPIN derivatives. In yet some further embodiments, the AAV vector used in the methods disclosed herein may comprise DARPin incorporated in the capsid. In yet some further embodiments, the AAV vector used herein may be a modified AAV vector having a capsid composed of DARPin that may replace the VP1 capsid protein or any fragments thereof.

In some embodiments, the modified AAV-VP1-DARPin CD8 used by the methods of the present disclosure may comprise the amino acid sequence as denoted by SEQ ID NO: 37, or any derivatives thereof. Still further, in some embodiments, the modified AAV-VP1-DARPin CD8 used by the methods of the present disclosure may be constructed using a construct that comprise the nucleic acid sequence as denoted by SEQ ID NO: 35, or any variants and homologs thereof.

In yet some further embodiments, the at least one exogenous nucleic acid sequence of interest used by the disclosed therapeutic methods, may further comprise an inducible suicide gene.

In some embodiments, the immune-related disorder treatable by the therapeutic methods of the present disclosure comprise at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition.

It should be appreciated that any of the disorders disclosed by the present disclosure in connection with other aspects, are applicable for any of the therapeutic methods of the present disclosure, specifically, any proliferative disorder disclosed herein in connection with TAAs, is also applicable in any of the methods of the present disclosure. In some embodiments, the proliferative disorder treatable by the disclosed therapeutic methods may be at least one neoplastic disorder. In yet some further embodiments, the neoplastic disorder may be any malignant or benign neoplastic disorder. In some embodiments the neoplastic disorder may be cancer. Thus, in some embodiments, the disclosed therapeutic methods may be used for treating cancer.

The methods of the present disclosure may be in some embodiments thereof, specifically suitable for disorders associated with the immune system.

Thus, in some specific embodiments, the subject treated by the systems and/or kits, compositions, methods and uses of the present disclosure, may be a subject suffering of an immune-related disorder. An “Immune-related disorder” or “Immune-mediated disorder”, as used herein encompasses any condition that is associated with the immune system of a subject, more specifically through inhibition and/or enhancement of the immune system, or that can be treated, prevented or ameliorated by reducing degradation of a certain component of the immune response in a subject, such as the adaptive or innate immune response. An immune-related disorder may include infectious conditions (e.g., viral, bacterial or fungal infections), inflammatory disease, autoimmune disorders, metabolic disorders and proliferative disorders, specifically, cancer. In some specific embodiments wherein the immune-related disorder or condition may be a primary or a secondary immunodeficiency.

In some specific embodiments, the methods of the present disclosure may be used for treating proliferative disorders. As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods of the present invention may be applicable for treatment of a patient suffering from any one of non-solid and solid tumors.

Malignancy, as contemplated in the present disclosure may be any one of carcinomas, melanomas, lymphomas, leukemias, myeloma and sarcomas.

Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes. Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further malignancies that may find utility in the present disclosure can comprise but are not limited to hematological malignancies (including lymphoma, leukemia and myeloproliferative disorders, as described above), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The present disclosure may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma. It should be appreciated that for treating cancer, the system/kit and cassette of the invention or any compositions or methods thereof may facilitate the in vivo targeted insertion of the nucleic acid sequence of interest, that encodes a therapeutic and/or regulatory molecule, for example, any receptor molecule such as the CAR and/or the TCR molecules as described herein before, that are specifically directed at TAAs. It should be understood that the invention thus encompasses the treatment of any of the malignancies described in this context, specifically any malignancies described in connection with associated TAAs as described herein before in connection with other aspects of the present disclosure.

In yet some further embodiments, and of particular relevance are patients' populations suffering from one of autoimmune disorders, that are also referred to as disorders of immune tolerance, when the immune system fails to properly distinguish between self and non-self-antigens.

Thus, according to some embodiments, the methods of the present disclosure may be used for the treatment of a patient suffering from any autoimmune disorder. In some specific embodiments, the methods of the present disclosure may be used for treating an autoimmune disease such as for example, but not limited to, inflammatory bowel disease (IBD), ulcerative colitis, Crohn's disease, fatty liver disease, Lymphocytic colitis, Ischaemic colitis, Diversion colitis, Behçet's syndrome, Indeterminate colitis, rheumatoid arthritis, systemic lupus erythematosus (SLE), Graft versus Host Disease (GvHD), Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, multiple sclerosis (MS), myasthenia gravis, plexus disorders e.g. acute brachial neuritis, polyglandular deficiency syndrome, primary biliary cirrhosis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, ankylosing spondylitis, pemphigus, bullous pemphigoid, dermatitis herpetiformis, psoriatic arthritis, reactive arthritis, and ankylosing spondylitis, inflammatory arthritis, including juvenile idiopathic arthritis, gout and pseudo gout, as well as arthritis associated with colitis or psoriasis, Pernicious anemia, some types of myopathy and Lyme disease (Late).

In yet some other embodiments, the methods of the invention may be also applicable for treating a subject suffering from an infectious disease. More specifically, such infectious disease may be any pathological disorder caused by a pathogen.

As used herein, the term “pathogen” refers to an infectious agent that causes a disease in a subject host. Pathogenic agents include prokaryotic microorganisms, lower eukaryotic microorganisms, complex eukaryotic organisms, viruses, fungi, mycoplasma, prions, parasites, for example, a parasitic protozoan, yeasts or a nematode.

In yet some further embodiments, the methods of the invention may be applicable in boosting the immune response against a pathogen that may be in further specific embodiment, a viral pathogen or a virus. The term “virus” as used herein, refers to obligate intracellular parasites of living but non-cellular nature, consisting of DNA or RNA and a protein coat. Viruses range in diameter from about 20 to about 300 nm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis. It should be noted that the term “viruses” is used in its broadest sense to include viruses of the families adenoviruses, papovaviruses, herpesviruses: simplex, varicella-zoster, Epstein-Barr (EBV), Cytomegalo virus (CMV), pox viruses: smallpox, vaccinia, hepatitis B (HBV), rhinoviruses, hepatitis A (HBA), poliovirus, rubella virus, hepatitis C (HBC), arboviruses, rabies virus, influenza viruses A and B, measles virus, mumps virus, human deficiency virus (HIV), HTLV I and II, Corona virus (CoV), Dengue, West Nile virus (WNV), Yellow fearer virus (YFV), Ebola and Zika virus.

In some further embodiments, the methods of the present disclosure may be applicable for immune-related disorder/s or condition that may be a pathologic condition caused by at least one pathogen. It should be appreciated that an infectious disease as used herein also encompasses any infectious disease caused by a pathogenic agent, specifically, a pathogen. Pathogenic agents include prokaryotic microorganisms, lower eukaryotic microorganisms, complex eukaryotic organisms, fungi, prions, parasites, yeasts, toxins and venoms. In yet some other specific embodiments, the systems and/or kits, compositions, methods and uses of the present disclosure may be applicable for treating an infectious disease caused by bacterial pathogens. More specifically, a prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

Particular species include Treponema pallidum, Borrelia burgdorferi, Neisseria gonorrhea, Neisseria meningitidis, Legionella pneumophila, Bordetella pertussis, Escherichia coli, Salmonella typhi, Salmonella typhimurium, Shigella dysenteriae, Klebsiella pneumoniae, Yersinia pestis, Vibrio cholerae, Hemophilus influenzae, Rickettsia rickettsii, Chlamydia trachomatis, Mycoplasma pneumoniae, Staphylococcus aureus, Streptococcus pneumoniae, Streptococcus pyogenes, Bacillus anthracis, Clostridium botulinum, Clostridium tetani, Clostridium perfringens, Corynebacterium diphtheriae, Proprionibacterium acnes, Mycobacterium tuberculosis, Mycobacterium leprae and Listeria monocytogenes.

A lower eukaryotic organism includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum, are also encompassed by the invention.

A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania.

More specifically, in certain embodiments the systems and/or kits, compositions, methods and uses of the present disclosure may be suitable for treating disorders caused by fungal pathogens. The term “fungi” (or a “fungus”), as used herein, refers to a division of eukaryotic organisms that grow in irregular masses, without roots, stems, or leaves, and are devoid of chlorophyll or other pigments capable of photosynthesis. Each organism (thallus) is unicellular to filamentous and possesses branched somatic structures (hyphae) surrounded by cell walls containing glucan or chitin or both and containing true nuclei. It should be noted that “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis.

As noted above, the present invention also provides for the systems and/or kits, compositions, methods and uses of the present disclosure for the treatment of a pathological disorder caused by “parasitic protozoan”, which refers to organisms formerly classified in the Kingdom “protozoa”. They include organisms classified in Amoebozoa, Excavata and Chromalveolata. Examples include Entamoeba histolytica, Plasmodium (some of which cause malaria), and Giardia lamblia. The term parasite includes, but not limited to, infections caused by somatic tapeworms, blood flukes, tissue roundworms, ameba, and Plasmodium, Trypanosoma, Leishmania, and Toxoplasma species.

As used herein, the term “nematode” refers to roundworms. Roundworms have tubular digestive systems with openings at both ends. Some examples of nematodes include, but are not limited to, basal order Monhysterida, the classes Dorylaimea, Enoplea and Secernentea and the “Chromadorea” assemblage.

In yet some further specific embodiments, the present disclosure provides compositions and methods for use in the treatment, prevention, amelioration or delay of the onset of a pathological disorder, wherein said pathological disorder is a result of a prion. As used herein, the term “prion” refers to an infectious agent composed of protein in a misfolded form. Prions are responsible for the transmissible spongiform encephalopathies in a variety of mammals, including bovine spongiform encephalopathy (BSE, also known as “mad cow disease”) in cattle and Creutzfeldt-Jakob disease (CJD) in humans. All known prion diseases affect the structure of the brain or other neural tissue, and all are currently untreatable and universally fatal.

It should be appreciated that an infectious disease as used herein also encompasses any pathologic condition caused by toxins and venom.

Still further, the methods of the present disclosure may offer a promising therapeutic modality for a variety of innate and acquired immunodeficiencies caused by immunosuppressive treatments (chemo- and radiotherapy), pathogenic infections, cancer and HSCT. More specifically, Immunodeficiency (or immune deficiency) is a state in which the immune system's ability to fight infectious disease and cancer is compromised or entirely absent. Most cases of immunodeficiency are acquired (“secondary”) due to extrinsic factors that affect the patient's immune system. Examples of these extrinsic factors include viral infection, specifically HIV, extremes of age, and environmental factors, such as nutrition. In the clinical setting, the immunosuppression by some drugs, such as steroids, can be either an adverse effect or the intended purpose of the treatment. Examples of such use are in organ transplant surgery as an anti-rejection measure and in patients suffering from an overactive immune system, as in autoimmune diseases. Immunodeficiency also decreases cancer immunosurveillance, in which the immune system scans the cells and kills neoplastic ones. Still further, Primary immunodeficiencies (PID), also termed innate immunodeficiencies, are disorders in which part of the organism immune system is missing or does not function normally. To be considered a primary immunodeficiency, the cause of the immune deficiency must not be caused by other disease, drug treatment, or environmental exposure to toxins). Most primary immune deficiencies are genetic disorders; the majority are diagnosed in children under the age of one, although milder forms may not be recognized until adulthood. While there are over 100 recognized PIDs, most are very rare. There are several types of immunodeficiency that include, Humoral immune deficiency (including B cell deficiency or dysfunction), which generally includes symptoms of hypogammaglobulinemia (decrease of one or more types of antibodies) with presentations including repeated mild respiratory infections, and/or agammaglobulinemia (lack of all or most antibody production) and results in frequent severe infections (mostly fatal); T cell deficiency, often causes secondary disorders such as acquired immune deficiency syndrome (AIDS); Granulocyte deficiency, including decreased numbers of granulocytes (called as granulocytopenia or, if absent, agranulocytosis) such as of neutrophil granulocytes (termed neutropenia); granulocyte deficiencies also include decreased function of individual granulocytes, such as in chronic granulomatous disease; Asplenia, where there is no function of the spleen; and Complement deficiency in which the function of the complement system is deficient. Secondary immunodeficiencies occur when the immune system is compromised due to environmental factors. Such factors include but are not limited to radiotherapy as well as chemotherapy. While often used as fundamental anti-cancer treatments, these modalities are known to suppress immune function, leaving patients with an increased risk of infection; indeed, infections were found to be a leading cause of patient death during cancer treatment. Neutropenia was specifically associated with vulnerability to life-threatening infections following chemotherapy and radiotherapy. In more specific embodiments, secondary immunodeficiency may be caused by at least one of chemotherapy, radiotherapy, biological therapy, bone marrow transplantation, gene therapy, adoptive cell transfer or any combinations thereof.

As described herein above, the invention provides in some aspects thereof therapeutic and prophylactic methods.

A further aspect of the present disclosure provides nucleic acid cassette/s and/or construct/s, for in vivo targeted insertion of at least one exogenous nucleic acid sequence of interest into at least one of the TRAC, and the TRBC loci of a cell of the T lineage in a mammalian subject. The cassette and/or delivery vector disclosed and used herein may comprise: (a) at least one nucleic acid molecule comprising the at least one exogenous nucleic acid sequence of interest, the sequence is flanked on at least one of the 5′ and 3′ thereof by at least one homology arm, for integration to the target site by homologous recombination; and (b) at least one nucleic acid sequence encoding at least one site specific nuclease.

The present disclosure involves the provision of a nucleic acid cassette and/or vector, that are used in the systems/kits, compositions, delivery vehicles, methods and uses as described herein. The nucleic acid cassettes of the present disclosure comprise in some embodiments the nucleic acid sequence of interest that encodes at least one therapeutic protein, for example, any receptor such as CAR, and/or exogeneous TCR. The nucleic acid cassette may comprise in addition or separately, nuclei acid sequences that encode at least one nuclease for the in vivo targeted insertion of the disclosed cassette. The term “nucleic acid cassette” refers to a polynucleotide sequence comprising at least one regulatory sequence operably linked to a sequence encoding a nucleic acid sequence of interest that may be a protein-encoding or non-coding sequence. In some embodiments, the nucleic acid cassettes contain at least one nucleic acid sequence/s of interest, e.g., a polynucleotide(s) of interest. In other embodiments, the nucleic acid cassette may contain one or more genetic elements e.g. expression control sequences and at least one nucleic acid sequence/s of interest. In some embodiments, the nucleic acid cassettes of the invention may be comprised within a vector. Still further, vectors may comprise one, two, three, four, five or more nucleic acid cassettes. The nucleic acid cassette may be positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. The cassette may have its 3′ and 5′ ends adapted for ready insertion into a vector, e.g., it may possess restriction endonuclease sites at each end. Still further, it should be appreciated that the nucleic acid cassette provided by the present disclosure (e.g., in the disclosed systems) that comprise at least one nucleic acid sequence of interest designed to be incorporated in a target locus may be also referred to herein as a donor nucleic acid. “Donor nucleic acid” or “donor nucleic acid molecule” is defined herein as any nucleic acid supplied to an organism or receptacle to be inserted, incorporated or recombined wholly or partially into the target sequence either by DNA repair mechanisms, homologous recombination (HR), or by non-homologous end-joining (NHEJ). A Donor nucleic acid molecule may be a nucleic acid sequence (either RNA or DNA or a modified nucleic acid or a combination thereof). Donor nucleic acid consisting of DNA or modified DNA may also be referred to as “donor DNA”. It should be appreciated that in some embodiments, the donor nucleic acid molecules of the systems of the present disclosure may be provided in one or more nucleic acid cassette. Since the disclosed donor nucleic acid molecule is incorporated into the target nucleic acid sequence via homologous recombination (HR), the donor nucleic acid sequence may also comprise, or specifically flanked by at least one homology arm, that displays complementarity to a nucleic acid sequence flanking the target site for incorporation.

In some embodiments, the nucleic acid cassette/s and/or construct/s, and/or vector/s and/or delivery vehicle/s used by the present disclosure may further comprise at least one genetic element. Such genetic element is at least one of: at least one IRES, at least one 2A peptide coding sequence, at least one promoter or any functional fragments thereof, at least one SD, at least one SA, at least one degron, at least one 3 frame stop, at least one protein stabilizing sequence, at least one signal peptide, at least one stop codon, at least one polyadenylation site, at least one transcription enhancer, at least one switch region, at least one mRNA stabilizing sequence and/or at least one protein stabilizing sequence. As noted above, in some embodiments, the cassettes used by the systems/kits and methods of the present disclosure may further comprise at least one IRES sequence.

By internal ribosome entry sequences (IRES) sequence is meant, a nucleotide sequence that allows for translation initiation in an end-independent manner, as part of the protein synthesis. IRES are able to recruit the eukaryotic ribosome to the mRNA and to provide two separate places where a ribosome may initiate translation on a single mRNA. IRES elements enable to create multigene, or polycistronic, messages since they are able to bypass the ribosome scanning model of 5′ methylated Cap dependent translation and begin translation at internal sites. Multiple open reading frames can be transcribed together, each separated by an IRES, creating polycistronic messages. By virtue of the IRES element, each open reading frame is accessible to ribosomes for efficient translation.

It should be noted that in some embodiments, the IRES sequence may be located 5′ to the nucleic acid sequence of interest. In some specific and non-limiting embodiments, an appropriate IRES that may be used by the invention may be the encephalomyocarditis virus (ECMV) IRES. In some particular and non-limiting embodiments, an ECMV IRES applicable in the present invention may comprise the nucleic acid sequence as denoted by SEQ ID NO. 2, or any homologs or variants thereof.

In yet some further embodiments, the cassette provided by the systems and/or kits, compositions, methods and uses of the present disclosure may further comprise at least one 2A peptide sequence. More specifically, a 2A peptide sequence or a CHYSEL site causes a eukaryotic ribosome to release the growing polypeptide chain, but continues translating, thereby giving rise to two separate polypeptides from a single translating ribosome. An expression cassette using a 2A peptide may be therefore used for two or more nucleic acid sequences of interest. In some embodiments, this sequence may be used to separate the coding region of two or more polypeptides encoded by two or more nucleic acid sequences of interest. As a non-limiting example, the sequence encoding the 2A peptide may be between a first coding region and a second coding region. In other embodiments, the 2A peptide may be used in the polynucleotide sequences or the cassettes of the present disclosure to produce two, three, four, five, six, seven, eight, nine, ten or more proteins, or any other product of the nucleic acid sequence of interest provided by the invention. In certain embodiments, a non-limiting example for 2A-peptide that may be used by the present disclosure may be the Picornaviruse 2A peptide (P2A). In some particular and non-limiting embodiments, a P2A peptide applicable in the present disclosure may be the Furin-GSG-P2A. In some embodiments, the P2A sequence used in the present disclosure provides a cleavage site for furin, that is a protease that belongs to the proprotein convertase (PC) family of proteases and is involved in the processing and activation of a wide range of proteins in human cells. Furin cleaves proteins at a specific amino acid sequence called the furin cleavage site, which is characterized by the motif R-X-K/R-R. In yet some specific embodiments, the GSG in the Furin-GSG-P2A used by the present disclosure refers to a short linker sequence of amino acids (Glycine-Serine-Glycine) that connects the Furin and P2A components. In yet some further embodiments, the Furin-GSG-P2A used in the present disclosure may comprise the nucleic acid sequence as denoted by SEQ ID NO: 3, or any variants and homologs thereof. Thus, in more specific embodiments the cassette of the present disclosure may comprise the nucleic acid sequence as denoted by SEQ ID NO: 3, or any variants or homologous thereof. In some further embodiments, the disclosed nuclei acid molecules and/or any cassettes thereof, may comprise at least one Furin-GSG sequence. In some embodiments, the Furin-GSG used in the present disclosure may comprise the nucleic acid sequence as denoted by SEQ ID NO: 26, or any variants and homologs thereof. This sequence encodes the Furin-GSG that comprises the amino acid sequence as denoted by SEQ ID NO: 28, or any variants or derivatives thereof. Thus, in more specific embodiments the cassette of the present disclosure may comprise the nucleic acid sequence as denoted by SEQ ID NO: 26, or any variants or homologous thereof.

In some further embodiments, the disclosed nuclei acid molecules and/or any cassettes thereof, may comprise at least one P2A sequence. In some embodiments, the P2A used in the present disclosure may comprise the nucleic acid sequence as denoted by SEQ ID NO: 27, or any variants and homologs thereof. This sequence encodes the P2A that comprises the amino acid sequence as denoted by SEQ ID NO: 29, or any variants or derivatives thereof. Thus, in more specific embodiments the cassette of the present disclosure may comprise the nucleic acid sequence as denoted by SEQ ID NO: 27, or any variants or homologous thereof.

Still further, in some embodiments, the nucleic acid cassette provided by the preset disclosure and used by the systems, constructs, vectors, compositions and methods of the disclosure may further comprise at least one promoter or any functional fragments thereof. As used herein, a “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present invention. In some embodiments, promoters applicable in the present disclosure may be either inducible or constitutive. In yet some further embodiments, a functional fragment of a promoter applicable in the methods and cassettes of the invention may be a minimal promoter. The term “minimal promoter” includes partial promoter sequences that define the start site of transcription for the linked sequence to be transcribed which by itself is not capable of initiating transcription. Thus, the activity of such a minimal promoter is dependent upon the binding of a transcriptional activator to an operatively linked regulatory sequence, e.g., enhancer. In certain embodiments a minimal promoter may be included in the cassettes of the invention. In some specific embodiment, the minimal promoter may be a T cell specific promoter, as disclosed herein after.

In yet some further embodiments, the systems/kits, cassettes, vectors and methods of the present disclosure may use an artificial or a synthetic promoter. In some embodiments, such promoter may be the JeT promoter. The JeT promoter is a recombinant promoter with transcriptional activity comparable to a number of strong mammalian promoters. The promoter consists of five key elements: (1) a TATA box; (2) a transcription initiation site (Inr); (3) a CAT consensus sequence in conjunction with (4) a CArG element and finally, (5) four Sp1 transcription binding sites (GGGCGG) arranged in two tandems.

A “constitutive promoter” refers to a promoter that allows for continual transcription of the coding sequence or gene under its control.

In yet some further embodiments, a promoter suitable in the cassette of the disclosure may be an inducible promoter. An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition.

It should be appreciated that the promoters suitable for the present invention may be either endogenous or heterologous. The phrase “endogenous promoter” includes a promoter that is naturally associated, e.g., in a wild-type organism, with an endogenous gene. Thus, in some specific embodiments, the cassette of the disclosure may comprise or operably linked to an endogenous promoter, for example, the endogenous promoter of the TCR beta or alpha chains. It should be appreciated that such endogenous promoter may be either ectopically added or may be used in its original endogenous location.

In yet some further embodiments, the cassette of the invention may comprise at least one heterologous promoter. The term “heterologous” includes a promoter from a different source or gene. It should be understood that in some embodiments, a promoter comprised within the nucleic acid cassette of the invention may be located 5′ to the nucleic acid sequence of interest. In some embodiments, relevant promoters that may be used by the methods and cassettes of the invention may include but are not limited to CMV promoter, SFFV promoter, EF1alpha promoter, AAT promoter, BgH promoter and any appropriate natural or artificial or synthetic promoter.

In some embodiments, a promoter useful in the present disclosure may be the JeT promoter. In more specific embodiments, such promoter may comprise the nucleic acid sequence as denoted by SEQ ID NO: 11, or any homologs or variants thereof.

Still further, it should be understood that various promoters as also applicable for the methods, systems vectors and cassettes of the present disclosure. Non-limiting embodiments for promoters applicable in the present disclosure may include, but are not limited to the PGK promoter, for example, the one denoted by SEQ ID NO: 12, or any homologs or variants thereof, the SFFV promoter, for example, the one denoted by SEQ ID NO: 13, or any homologs or variants thereof, the CMV promoter, for example, as denoted by SEQ ID NO: 14, or any homologs or variants thereof, the EF1A promoter, as denoted by SEQ ID NO: 15, or any homologs or variants thereof, the CD3 promoter, specifically, the promoter comprising the nucleic acid sequence as denoted by SEQ ID NO: 16, or any homologs or variants thereof, or any appropriate promoter available.

In some embodiments, the targeted cassettes of the present disclosure may include 2A peptide or an IRES sequence upstream to the nucleic acid sequence of interest encodes in some embodiments, at least one therapeutic and/or modulatory molecule, e.g., the CAR and/or TCR encoding sequence, in order to avoid formation of protein fusions with preceding segments while still utilizing the strong endogenous promoter. The IRES may be in some embodiments, preceded by a 3-frame stop to prevent ongoing translation. In some embodiments, the cassette/s, and/or construct/s, and/or vector/s may encode in some embodiments several peptides (at least one therapeutic and/or modulatory molecule, e.g. CAR and/or TCR) and these are separated by 2A peptides.

In yet some further embodiments, the cassettes provided by the present disclosure and used by the systems and/or kits, compositions, methods and uses of the present disclosure, may further comprise at least one degron sequence. Degrons are readily understood by one of ordinary skill in the art to be amino acid sequences that control the stability of the protein of which they are part. In some embodiments, a suitable degron comprised within the nucleic acid cassette of the invention may be constitutive. In yet some further embodiments, the degron may exert its influence on protein in an inducible manner. In some embodiments, the degron sequence may be located 5′ to the nucleic acid sequence of interest.

In yet some further embodiments, the nucleic acid cassette provided by the present disclosure and by the methods and compositions of the invention may comprise at least one signal peptide. “Signal peptide”, as used herein, shall mean a peptide chain (of about 3-60 amino acids long) that directs the post-translational transport of a protein to the endoplasmic reticulum and may be cleaved off.

In some embodiments, the signal peptide may be located 5′ to the nucleic acid sequence of interest. In some further embodiments, the nucleic acid cassette provided by the systems and/or kits, compositions, methods and uses of the present disclosure may comprise at least one mRNA stabilizing sequence. As used herein, a mRNA stabilizing sequence refers to a nucleic acid sequence that enables to extend the lifetime of a mRNA strand. Non limiting examples of mRNA stabilizing elements may include Polyadenylation, 3′ untranslated regions (3′-UT) such as histone mRNA 3′-terminal stem-loop, AU-rich elements (AUREs), Iron-responsive element and Long-range stem loop of insulin-like growth factor II (IGF II), mRNA cap.

In some embodiments, the mRNA stabilizing sequence may be located 3′ to the nucleic acid sequence of interest.

In yet some further embodiments, the cassette provided by the systems and/or kits, compositions, methods and uses of the present disclosure, may comprise at least one stop codon. A stop codon (or termination codon) is a nucleotide triplet within messenger RNA that signals a termination of translation into proteins. Stop codons signal the termination of this process by binding release factors, which cause the ribosomal subunits to disassociate, releasing the amino acid chain. There are three different stop codons in RNA; UAG (“amber”), UAA (“ochre”), UGA (“opal”), in DNA; TAG (“amber”), TAA (“ochre”), TGA (“opal” or “umber”). It should be noted that in some embodiments, the stop codon may be located 3′ to the nucleic acid sequence of interest.

In yet some further embodiments, the cassette provided by the systems and/or kits, compositions, methods and uses of the present disclosure may comprise at least one 3-frame stop codon sequence. More specifically, the cassette may comprise protein translation stop codons in each frame of translation, so that translation from the transcripts of any nucleic acid sequence of interest is halted at the point of insertion. Each translation stop sequence (known henceforth as a “3 frame stop codon sequence”) carries stop codons in all 3 frames of translation. In some embodiments, the 3 frame stop codon sequence may be located 5′ to the nucleic acid sequence of interest.

Still further, in certain embodiments, the cassette provided by the present disclosure and by the systems and/or kits, compositions, methods and uses of the present disclosure, may comprise a nucleic acid sequence encoding at least one protein stabilizing sequence. A protein stabilizing sequence relates to an amino acid sequence useful for stabilization of otherwise unstable proteins, particularly proteolytically sensitive proteins. The stabilization sequence may include a limited number of amino acids ranging from about ten to about 50 residues. The amino acids are such that the secondary and tertiary structure assumes the form of an outwardly directed, properly aligned hydrophobic face and a positively charged polar face.

In some embodiments, the protein stabilizing sequence may be located 5′ to the nucleic acid sequence of interest.

Still further, in some embodiments, the cassette provided by the present disclosure and by the systems and/or kits, compositions, methods and uses of the present disclosure, may comprise at least one polyadenylation sequence. Polyadenylation is the addition of a poly(A) tail to a messenger RNA consisting of multiple adenosine monophosphates. In eukaryotes, polyadenylation is part of the process that produces mature messenger RNA (mRNA) for translation. The process of polyadenylation begins as the transcription of a gene terminates. The 3′-most segment of the newly made pre-mRNA is first cleaved off by a set of proteins; these proteins then synthesize the poly(A) tail at the RNA's 3′ end. The polyadenylation signal varies between groups of eukaryotes. Most human polyadenylation sites contain the AAUAAA sequence. In some embodiments, the polyadenylation sequence may be located 3′ to the nucleic acid sequence of interest.

In some embodiments, the polyA element applicable in the present disclosure and in the cassettes, the systems and/or kits, compositions, methods and uses of the present disclosure, may be the BGH polyA. More specifically, such an element may comprise the nucleic acid sequence as denoted by SEQ ID NO: 17, and any variants and homologs thereof. Still further, in some embodiments, the polyA element applicable in the present disclosure may be the sv40 polyA. More specifically, such element may comprise the nucleic acid sequence as denoted by SEQ ID NO: 18, and any variants and homologs thereof.

In certain embodiments, the cassette provided by the present disclosure and by the systems and/or kits, compositions, methods and uses of the present disclosure, may comprise at least one splice donor site. In yet some further embodiments, the cassette provided by the methods of the invention may comprise at least one splice acceptor site. More specifically, splicing is the editing of the nascent precursor messenger RNA (pre-mRNA) transcript. After splicing, introns are removed, and exons are joined together. Introns often reside within the sequence of eukaryotic protein-coding genes. Within the intron, a donor site (5′ end of the intron), a branch site (near the 3′ end of the intron) and an acceptor site (3′ end of the intron) are required for splicing. The splice donor site includes an almost invariant sequence GU at the 5′ end of the intron, within a larger, less highly conserved region. The splice acceptor site at the 3′ end of the intron terminates the intron with an almost invariant AG sequence. Upstream (5′-ward) from the AG there is a region high in pyrimidines (C and U), or polypyrimidine tract. Further upstream from the polypyrimidine tract is the branch point, which includes an adenine nucleotide involved in lariat formation.

Still further, in some embodiments, the cassette provided by the present disclosure and by the systems and/or kits, compositions, methods and uses of the present disclosure, may comprise at least one enhancer. A transcription enhancer is a short (50-1500 bp) region of DNA that can be bound by proteins (activators) to increase the likelihood that transcription of a particular gene will occur. These proteins are usually referred to as transcription factors. Enhancers are generally cis acting but can also be trans-acting (acting away from the gene) and can be located up to 1 million bp (1,000,000 bp) away from the gene and can be upstream or downstream from the start site, and either in the forward or backward direction. There are hundreds of thousands of enhancers in the human genome. The invention thus encompasses in some embodiments thereof the use of any suitable enhancer. In some embodiments, the enhancer sequence may be located 3′ to the nucleic acid sequence of interest.

It should be noted that in some embodiments, each of the indicated genetic elements may be located either 5′ or 3′, or both, at the 5′ and 3′ (or in other words upstream and/or downstream), to the nucleic acid sequence of interest in the cassette provided by the methods of the invention. It should be noted that the terms used herein “5” or “upstream” and “3′” or “downstream” both refer to a relative position in DNA or RNA. Each strand of DNA or RNA has a 5′ end and a 3′ end, so named for the carbon position on the deoxyribose (or ribose) ring. By convention, upstream and downstream relate to the 5′ to 3′ direction in which RNA transcription takes place. Upstream is toward the 5′ end of the DNA or RNA molecule and downstream is toward the 3′ end. When considering double-stranded DNA, upstream is toward the 5′ end of the protein coding strand for the gene in question and downstream is toward the 3′ end. Due to the anti-parallel nature of DNA, this means the 3′ end of the mRNA template strand is upstream of the gene and the 5′ end is downstream. As used herein, the term “5” refers to the part of the strand that is closer to the 5′ end or 5′ terminus, i.e. to the extremity of the DNA or RNA strand that has a phosphate group attached to the fifth carbon in the sugar-ring of the deoxyribose or ribose at its terminus. Furthermore, the term “3” refers to the part of the strand that is closer to the 3′ end or 3′ terminus, i.e. to the extremity of the DNA or RNA strand that has a hydroxyl group linked to the 3rd carbon in the sugar-ring of the deoxyribose or ribose at its terminus.

In certain embodiments, the cassette provided by the present disclosure and by the systems and/or kits, compositions, methods and uses of the present disclosure, may comprise two or more nucleic acid sequences of interest separated by at least one genetic element, specifically, any of the gene elements disclosed above. In more specific embodiments such genetic element may be at least one of: an IRES, a 2A peptide coding sequence and a promoter and any functional fragments thereof. Thus, in some embodiments the cassettes of the present disclosure may comprise at least two, three, four, five, six, seven, eight, nine, ten or more, fifteen, twenty, twenty-five, thirty, thirty-five, forty, forty five, fifty, fifty five, sixty, sixty five, seventy, seventy five, eighty, eighty five, ninety, ninety five, hundred or more nucleic acid sequences of interest separated by at least one genetic element.

A “coding sequence”, as used herein, is a nucleic acid sequence which is transcribed and translated into a polypeptide in vivo or in vitro when placed under the control of appropriate regulatory sequences. The boundaries of the coding sequence are determined by a start codon at the 5′ (amino) terminus and a translation stop codon at the 3′ (carboxyl) terminus.

All elements comprised within the cassette of the invention are operably linked together. The term “operably linked”, as used in reference to a regulatory sequence and a structural nucleotide sequence, means that the nucleic acid sequences are linked in a manner that enables regulated expression of the linked structural nucleotide sequence.

In some embodiments, the cassette/s and/or construct/s, and/or vector/s and/or delivery vehicle/s provided and used by the present disclosure may comprise at least one nucleic acid sequence of interest. In some embodiments, the nucleic acid sequence of interest encodes at least one therapeutic and/or modulatory molecule. In some embodiments, the nucleic acid sequence of interest encodes at least one therapeutic protein. In more specific embodiments, the therapeutic protein may be a receptor molecule. Still further in some embodiments, such receptor molecule may be a CAR molecule and/or a TCR molecule. Accordingly, in some embodiments the nucleic acid sequence may be flanked by at least one of the 5′ and/or 3′ ends thereof by a right homology arm and/or a left homology arm. The cassette/s and/or construct/s, and/or vector/s and/or delivery vehicle/s provided and used by the present disclosure further comprise at least one nucleic acid sequence encoding the ARCUS nuclease.

In some embodiments the present disclosure provides at least one nucleic acid cassette that allows an in vivo targeted insertion of a nucleic acid sequence into a target locus in a cell of the T lineage. In some embodiments, the disclosed nucleic acid cassette may comprise from the 5′ end thereof, a left homology arm followed by a splice acceptor site (SA), furin-2A, the nucleic acid sequence of interest that in some embodiments encode at least one therapeutic and/or modulatory molecule, followed by a polyA sequence and flanked by a right homology arm, the cassette further comprises an exogenous promoter, an NLS coding sequence and a nucleic acid sequence encoding at least one nuclease followed by a polyA sequence. A schematic presentation of such cassette is disclosed by FIG. 1A(i).

In some specific embodiments, the nucleic acid sequence of interest flanked by both homology arms, encodes at least one therapeutic and/or modulatory molecule. In some embodiments, the nucleic acid sequence of interest encodes at least one therapeutic protein. In more specific embodiments, the therapeutic protein may be a receptor molecule. Still further in some embodiments, such receptor molecule may be a CAR molecule and/or a TCR molecule. In some specific embodiments, the nucleic acid sequence of interest encodes at least one CAR molecule, for example, a CAR molecule directed at the CD19 antigen. Still further, in some embodiments the hCD19 CAR molecule may comprise the amino acid sequence as denoted by SEQ ID NO: 4. In yet some further embodiments, the CAR molecule of the cassettes disclosed in the present disclosure may comprise at least one nucleic acid sequence encoding the hCD19 CAR molecule. In some embodiments, such sequence may comprise the nucleic acid sequence as denoted by SEQ ID NO: 5, or any variants and homologs thereof. Still further, in some embodiments, the nuclease encoded by the cassette of the present disclosure may be the ARCUS nuclease. In some specific embodiments, the ARCUS nuclease may specifically recognize a target locus within the TRAC gene. Still further, in some embodiments the ARCUS disclosed by the present disclosure may comprise the amino acid sequence as denoted by SEQ ID NO: 7, or any derivatives and variants thereof. Still further, in some embodiments, the ARCUS may be encoded by the nucleic acid sequence as denoted by SEQ ID NO: 6, or any homologs or variants thereof. Thus, in some particular embodiments, the cassette of the present disclosure may further comprise the nucleic acid sequence as denoted by SEQ ID NO: 6. Still further, in some embodiments, the ARCUS provided in the disclosed cassette is under the exogeneous JeT promoter. In more specific embodiments, the cassette of the present disclosure may comprise the nucleic acid sequence as denoted by SEQ ID NO: 19, or any variants and derivatives thereof.

In yet some alternative embodiments, the nucleic acid cassette of the present disclosure may comprise from the 5′ end thereof, a left homology arm followed by a splice acceptor site (SA), followed by a polyA sequence, a JeT promoter and at least one nucleic acid sequence encoding the therapeutic molecule of interest (e.g., CAR molecule, specifically, the hCD19 CAR molecule, followed by a Furin-2A sequence, a splice donor site (SD), and a right homology arm, followed by a second SA, NLS sequence and a sequence encoding the nuclease (e.g., ARCUS) followed by a poly A sequence. A schematic presentation of such cassette is disclosed by FIG. 1A(ii).

In yet some other alternative embodiments, the nucleic acid cassette of the present disclosure may comprise from the 5′ end thereof, an exogenous promoter sequence (e.g. JeT promoter), followed by NLS and a sequence encoding at least one nuclease (e.g., the ARCUS), followed by a Furin-2A sequence, a left homology arm followed by a splice acceptor site (SA), followed by at least one nucleic acid sequence encoding the therapeutic molecule of interest (e.g., CAR molecule, specifically, the hCD19 CAR molecule, followed a poly A sequence and a right homology arm sequence. A schematic presentation of such cassette is disclosed by FIG. 1A(iii).

In some specific embodiments of the cassettes disclosed herein, the nucleic acid sequence of interest encodes at least one CAR molecule, for example, a CAR molecule directed at the CD19 antigen. Still further, in some embodiments the hCD19 CAR molecule may comprise the amino acid sequence as denoted by SEQ ID NO: 4. In yet some further embodiments, the CAR molecule of the cassettes disclosed in the present disclosure may comprise at least one nucleic acid sequence encoding the hCD19 CAR molecule. In some embodiments, such sequence may comprise the nucleic acid sequence as denoted by SEQ ID NO: 5, or any variants and homologs thereof. Still further, in some embodiments, the nuclease encoded by the cassette of the present disclosure may be the ARCUS nuclease. In some specific embodiments, the ARCUS nuclease may specifically recognize a target locus within the TRAC gene. Still further, in some embodiments the ARCUS disclosed by the present disclosure may comprise the amino acid sequence as denoted by SEQ ID NO: 7, or any derivatives and variants thereof. Still further, in some embodiments, the ARCUS may be encoded by the nucleic acid sequence as denoted by SEQ ID NO: 6, or any homologs or variants thereof. Thus, in some particular embodiments, the cassette of the present disclosure may further comprise the nucleic acid sequence as denoted by SEQ ID NO: 6.

In some specific and non-limiting embodiments, the cassette/s and/or construct/s, and/or vector/s and/or delivery vehicle/s of the present disclosure may comprise the nucleic acid sequence as denoted by SEQ ID NO: 19, or any variants, homologs or derivatives thereof. More specifically, in some specific and non-limiting embodiments, the cassette and/or vector disclosed by the present disclosure and by the systems and/or kits, compositions, methods and uses of the present disclosure, may comprise nucleic acid sequences encoding the ARCUS as denoted by the nucleic acid sequence of SEQ ID NO: 6, or any homologs or derivatives thereof, the nucleic acid sequence encoding the anti-CD19 CAR T molecule as denoted by the nucleic acid sequence of SEQ ID NO: 5, or any homologs or derivatives thereof, flanked by the right and left homology arms of SEQ ID NO: 10 (RHA), and SEQ ID NO: 9 (LHA), respectively, for example, as illustrated by FIG. 1A. In some specific embodiments, such cassette and/or vector comprises the nucleic acid sequence as denoted by SEQ ID NO: 19, or any variants, homologs and derivatives thereof.

The present disclosure provides nucleic acid cassette and methods using the cassette. The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. Preparation of nucleic acids is well known in the art.

Still further, it should be understood that the invention encompasses as additional aspects thereof any vector or vehicle that comprise any of the cassettes described by the invention. Still further, the invention also provides any cell, specifically a mammalian host cell that express the cassettes of the invention or any of the vectors or vehicles described by the invention. In some specific embodiments, such host cells may be immune-related cells, specifically lymphocytes, more specifically, lymphocytes either obtained in some embodiments from a healthy subject or in other embodiments, obtained from a subject suffering from an immune-related disorder. Specific embodiments relate to T and NK T lymphocytes that are transduced or transfected with the cassettes of the invention or any vector or vehicle thereof. It should be understood that any of the cells, vectors and subjects described by the invention also apply to these aspects as well.

A further aspect provided by the present disclosure relates to a therapeutically effective amount of: (a) at least one nucleic acid molecule comprising at least one exogenous nucleic acid sequence of interest, and/or at least one cassette/s and/or construct/s thereof. The sequence is flanked on at least one of the 5′ and 3′ thereof by at least one homology arm, for integration by homologous recombination to at least one target locus of a cell of the T lineage in the subject. It should be noted that the at least one nucleic acid molecule and/or at least one cassette/s and/or construct/s is comprised within at least one AAV vector and/or AAV-like vector; and (b), at least one site specific nuclease, or at least one nucleic acid sequence encoding the nuclease or cassette/s and/or construct/s, and/or vector/s and/or delivery vehicle/s comprising the at least one nuclease or the nucleic acid sequence encoding the nuclease. Alternatively (c), any system, delivery vehicle, matrix, nano- or micro-particle and/or composition comprising (a) and/or (b) or any cassette/s and/or construct/s, comprising (a) and (b) or (a) or (b); for use in a method for treating, preventing, ameliorating, inhibiting or delaying the onset of an immune-related disorder in a mammalian subject.

In some embodiments, the target locus targeted by the disclosed systems used herein, may be at least one of TCR α chain locus, TCRβ chain locus, TCRγ chain locus and the TCRS chain locus.

It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease.

The term “treatment or prevention” as used herein, refers to the complete range of therapeutically positive effects of administrating to a subject including inhibition, reduction of, alleviation of, and relief from, an immune-related condition and illness, immune-related symptoms or undesired side effects or immune-related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the compositions and methods according to the present disclosure, wherein the improvement may be manifested in the forms of inhibition of pathologic processes associated with the immune-related disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the disclosure described herein.

The terms “delay”, “delaying the onset”, “retard” and all variations thereof are intended to encompass the slowing of the progress and/or exacerbation of a disorder associated with the immune-related disorders and their symptoms slowing their progress, further exacerbation or development, so as to appear later than in the absence of the treatment according to the invention.

As indicated above, the systems and/or kits, compositions, methods and uses of the present disclosure may be used for the treatment of a “pathological disorder”, specifically, immune-related disorders as specified by the invention, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein.

It should be appreciated that any of the systems and/or kits, compositions, methods and uses of the present disclosure may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The present invention relates to the treatment of subjects or patients in need thereof. By “patient” or “subject in need” it is meant any organism who may be affected by the above-mentioned conditions, and to whom the therapeutic and prophylactic methods herein described are desired, including humans, domestic and non-domestic mammals such as canine and feline subjects, bovine, simian, equine and rodents, specifically, murine subjects. More specifically, the methods of the invention are intended for mammals. By “mammalian subject” means any mammal for which the proposed therapy is desired, including human, livestock, equine, canine, and feline subjects, most specifically humans.

It should be appreciated that any of the nucleic acid cassettes described herein as specific and non-limiting embodiments, are encompassed by the invention and may be used in any of the methods described herein before and in any of the compositions described herein after.

In a further aspect thereof, the present disclosure provides an effective amount of:

(a) at least one nucleic acid molecule comprising at least one exogenous nucleic acid sequence of interest, and/or at least one cassette/s and/or construct/s thereof. The sequence is flanked on at least one of the 5′ and 3′ thereof by at least one homology arm, for integration by homologous recombination to at least one target locus of a cell of the T lineage in the subject. It should be noted that the at least one nucleic acid molecule and/or at least one cassette/s and/or construct/s is comprised within at least one AAV vector and/or AAV-like vector; and (b), at least one site specific nuclease, or at least one nucleic acid sequence encoding the nuclease or cassette/s and/or construct/s, and/or vector/s and/or delivery vehicle/s comprising the at least one nuclease or the nucleic acid sequence encoding the nuclease. Alternatively (c), any system, delivery vehicle, matrix, nano- or micro-particle and/or composition comprising (a) and/or (b) or any cassette/s and/or construct/s, comprising (a) and (b) or (a) or (b); for use in a method for in vivo targeted insertion of the at least one exogenous nucleic acid sequence of interest into a target genomic locus of a cell of the T lineage, in a mammalian subject.

In some embodiments, the target locus targeted by the disclosed systems used herein, may be at least one of TCR α chain locus, TCRβ chain locus, TCRγ chain locus and the TCRS chain locus.

A further aspect of the present disclosure relates to an in vivo genetically engineered cell of the T cell lineage, any population of cells comprising at least one the genetically modified cell, or any composition comprising the cell or population of cells. The cell comprises at least one modified TRAC and/or TRBC loci comprising at least one exogenous nucleic acid sequence of interest. It should be noted that the cell was genetically modified by at least one nucleic acid cassette or vector comprising: (a) at least one nucleic acid molecule comprising the at least one exogenous nucleic acid sequence of interest, and/or at least one cassette/s and/or construct/s thereof. The sequence may be flanked on at least one of the 5′ and 3′ thereof by at least one homology arm, for integration to the TRAC and/or TRBC loci by homologous recombination. It should be noted that the at least one nucleic acid molecule and/or at least one cassette/s and/or construct/s is comprised within at least one AAV vector and/or AAV-like vector; and (b) at least one nucleic acid sequence encoding at least one site specific nuclease.

Still further, it must be appreciated that the invention further provides any host cell expressing the nucleic acid cassettes disclosed by the present disclosure. It should be understood that the cell is engineered in vivo, in the body of the mammalian subject.

In some specific embodiments, the cassette of the invention introduces the nucleic acid of interest in a target locus of a mammalian cell that is considered herein as a host cell. The term “host cell” includes a cell into which a heterologous (e.g., exogenous) nucleic acid or protein has been introduced. Persons of skill upon reading this disclosure will understand that such terms refer not only to the particular subject cell, but also is used to refer to the progeny of such a cell, e.g., progeny of the T cells divided in vivo in the subject. Because certain modifications may occur in succeeding generations due to either mutation or environmental influences, such progeny may not, in fact, be identical to the parent cell, but are still included within the scope of the term “host cell”.

A further aspect of the present disclosure relates to a composition comprising an effective amount of at least one system or nucleic acid cassette/s and/or construct/s, and/or vector/s and/or delivery vehicle/s, or any matrix, nano- or micro-particle thereof, for in vivo targeted insertion of at least one exogenous nucleic acid sequence of interest into a target genomic locus of a cell of the T lineage in a mammalian subject. The system and/or nucleic acid cassette/s and/or construct/s, and/or vector/s and/or delivery vehicle/s may comprise: (a) at least one nucleic acid molecule comprising the at least one exogenous nucleic acid sequence of interest, and/or at least one cassette/s and/or construct/s thereof. The sequence is flanked on at least one of the 5′ and 3′ thereof by at least one homology arm, for integration to the target locus by homologous recombination. It should be noted that the at least one nucleic acid molecule and/or at least one cassette/s and/or construct/s is comprised within at least one AAV vector and/or AAV-like vector; and (b) at least one site specific nuclease or at least one nucleic acid sequence encoding the site specific nuclease. The composition optionally further comprises at least one pharmaceutically acceptable carrier/s, excipient/s, auxiliaries, and/or diluent/s.

In some embodiments, the target locus is at least one of TCR α chain locus, TCRβ chain locus, TCRγ chain locus and the TCRδ chain locus.

In some embodiments, the system comprised in the disclosed composition is any of the systems described by the invention, and the nucleic acid cassette or vector used for the composition of the present disclosure is as defined by the present invention.

The compositions of the invention may comprise an effective amount of the system/kit, compositions and cassette of the invention or of any vector thereof or of any cell comprising the same. The term “effective amount” relates to the amount of an active agent present in a composition, specifically, the cassette of the present disclosure as described herein that is needed to provide a desired level of active agent in the bloodstream or at the site of action in an individual (e.g., the thymus or bone marrow) to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein. An “effective amount” of the in vivo targeting systems and/or kits, compositions, methods and uses of the present disclosure, compositions and cassette of the invention can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

The pharmaceutical compositions of the invention can be administered and dosed by the methods of the invention, in accordance with good medical practice, systemically, for example by parenteral, e.g. intrathymic, into the bone marrow and intravenous. It should be noted however that the invention may further encompass additional administration modes. In other examples, the pharmaceutical composition can be introduced to a site by any suitable route including intraperitoneal, subcutaneous, transcutaneous, topical, intramuscular, intraarticular, subconjunctival, or mucosal, e.g. oral, intranasal, or intraocular administration.

Local administration to the area in need of treatment may be achieved by, for example, by local infusion during surgery, topical application, direct injection into the specific organ (thymus, bone marrow, spleen, lymph nodes), etc. More specifically, the compositions used in any of the methods of the invention, described herein before, may be adapted for administration by intravenous, intrathymic, bone marrow, splenic administration and injection to lymph nodes, parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

In yet some further embodiments, the composition of the present disclosure may optionally further comprise at least one of pharmaceutically acceptable carrier/s, excipient/s, additive/s diluent/s and adjuvant/s.

Still further, pharmaceutical preparations are compositions that include one or more targeting cassettes present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols.

Still further, the composition/s of the present disclosure and any components thereof may be applied as a single daily dose or multiple daily doses, preferably, every 1 to 7 days. It is specifically contemplated that such application may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month. The application of the systems and/or kits, compositions, methods and uses of the present disclosure or of any component thereof may last up to a day, two days, three days, four days, five days, six days, a week, two weeks, three weeks, four weeks, a month, two months three months or even more. Specifically, application may last from one day to one month. Most specifically, application may last from one day to 7 days.

The term “effective amount” relates to the amount of an active agent present in a composition, specifically, the nucleic acid molecules, vectors and/or cassette of the invention as described herein that is needed to provide a desired level of active agent in the bloodstream or at the site of action in an individual (e.g., the thymus or bone marrow) to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein. An “effective amount” of the nucleic acid molecule/s of the invention or any cassette of the invention can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

The pharmaceutical compositions of the invention can be administered and dosed by the methods of the invention, in accordance with good medical practice, systemically, for example by parenteral, e.g., intrathymic, into the bone marrow and intravenous. It should be noted however that the invention may further encompass additional administration modes. In other examples, the pharmaceutical composition can be introduced to a site by any suitable route including intraperitoneal, subcutaneous, transcutaneous, topical, intramuscular, intraarticular, subconjunctival, or mucosal, e.g., oral, intranasal, or intraocular administration.

Local administration to the area in need of treatment may be achieved by, for example, by local infusion during surgery, topical application, direct injection into the specific organ (thymus, bone marrow, spleen, lymph nodes), etc. More specifically, the compositions used in any of the methods of the invention, described herein, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, the systems/kits, cassettes compositions of the invention with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations. It should be understood that in addition to the ingredients particularly mentioned above, the formulations may also include other agents conventional in the art having regard to the type of formulation in question. Still further, pharmaceutical preparations are compositions that include one or more nucleic acid molecules, vectors and/or cassette present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle”, when referred to the compositions in the present aspect, refers to a diluent, adjuvant, excipient, or carrier with which a compound of the invention is formulated for administration to a mammal. Such pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like.

In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the nucleic acid molecule/s encoding the CARs and/or TCRs of the invention, can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity, or it may be formulated for sustained release.

It should be understood that in some embodiments, where the nucleic acid sequence of interest encodes a receptor molecule such as CAR and/or TCR, the disclosed systems, viral vectors, methods, compositions and uses of the present disclosure, may lead to higher rate of in vivo engineered cells, for example, CAR T cells which harbor higher percentage of naive and central memory phenotype. In some embodiments, the central memory phenotype may comprise increased expression of at least one of the following markers, CD62L+/CCR7+/CD45RA+ or CD62L+/CCR7+/CD45RO+. Still further, in some embodiments, this phenotype may be associated with greater in vivo antitumor activity and less exhaustion markers such as PD1, LAG3 and TIM3. Thus, in yet some further embodiments, the cells of the T lineage that are in vivo genetically engineered by the systems, viral vectors, methods, compositions and uses of the present disclosure, may display reduced exhaustion. More specifically, T cell Exhaustion as used herein refers to a state of T cell dysfunction that arises during many chronic infections and cancer. It is defined by poor effector function, sustained expression of inhibitory receptors and a transcriptional state distinct from that of functional effector or memory T cells. By “dysfunction” here it is understood that some T cells, after activation and proliferation, do not fulfill the functions they are expected to perform as effector T cells typically, they fail to eliminate cancerous or infected cells and control the tumor or the virus respectfully. As originally described, antigen-specific T cells become “dysfunctional” during the chronic phase of high viral load infections, with progressive loss of interleukin (IL-2), then tumor necrosis factor alpha (TNFα), and, finally, interferon gamma (IFNγ). Thus, in some embodiments, the cells of the T lineage that are in vivo genetically engineered by the systems, viral vectors, methods, compositions and uses of the present disclosure display, or are characterized by reduced expression of exhaustion markers. More specifically, in some embodiments, the exhaustion markers may be at least one of Programmed Death-1 receptor (PD-1), Lymphocyte activation gene-3 (LAG-3, is also named CD223 or FDC protein), T-cell immunoglobulin and mucin-domain containing-3 (TIM3), T cell immunoreceptor with Ig and ITIM domains (TIGIT). Still further, in some additional and non-limiting embodiments, exhaustion markers applicable in the present disclosure include inducible T-cell co-stimulator (ICOS), cytotoxic T-lymphocyte-associated protein-4 (CTLA-4), CD244 (2B4), CD160, killer cell lectin-like receptor subfamily G member 1 (KLRG1), and the like.

Increase, as used herein, in connection with various improved properties of the cells of the T lineage that are in vivo genetically engineered by the systems, viral vectors, methods, compositions and uses of the present disclosure, is meant that such increase or enhancement may be an increase or elevation of the indicated activity (e.g., specificity, central memory phenotype, and/or expression of activation markers and the like), of between about 1% to 100%, specifically, 5% to 100% of the indicated parameter, more specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more. In yet some further embodiments, the terms “inhibition”, “moderation”, “reduction”, “decrease” or “attenuation” as referred to herein with respect to the various properties of cells of the T lineage that are in vivo genetically engineered by the systems, viral vectors, methods, compositions and uses of the present disclosure, (e.g., expression of exhaustion markers), relate to the retardation, restraining or reduction of the indicated parameter by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more. More specifically, the terms “increase”, “augmentation” and “enhancement” as used herein relate to the act of becoming progressively greater in size, amount, number, or intensity. Alternatively, “inhibition”, “moderation”, “reduction”, “decrease” or “attenuation” as used herein relate to the act of becoming progressively smaller in size, amount, number, or intensity. Particularly, an increase or alternatively, decrease of 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more.

Still further, it should be appreciated that in vivo, site-specific gene targeting in T cells is non-trivial and was not performed prior to the time of the present invention. To achieve in vivo site-specific gene targeting in T cells—enough T cells should be transduced by both a nuclease and a CAR/TCR homologous recombination donor.

Still further, in some embodiments, single vector with the small nuclease, ARCUS, may the best shot at this challenging goal (although in some embodiments, the present disclosure further encompasses the use of dual vector approaches with one vector coding for a CRISPR Cas9 system and the other vector coding for the CAR/TCR homologous recombination donor.

It should be further appreciated that the present disclosure provides transducing T cells in vivo with an AAV vector coding for a CAR/TCR homologous recombination donor and with an AAV coding for a nuclease. In some embodiments, this is accomplished using a single vector, which codes for both a nuclease and CAR/TCR homologous recombination donor. This is a major challenge given the small size of the AAV vector.

There are currently six approved FDA drugs which are AAV based, and dozens ongoing clinical trials. On the other hand, AAV has a limited coding capacity of 4.4 kb between the ITRs. Therefore, combining both a nuclease and a homologous recombination donor sequence in a single vector is firstly disclosed by the present disclosure.

In yet some further embodiments, the ARCUS cleavage rates may be optimized by modulating several parameters. The Arcus cleavage activity was higher when an SV40 NLS was added on the N terminus, although the Arcus is small enough to pass theoretically through the nucleus pores. The NLS used, its location, number of repeats, may further be modulated for optimizing the in vivo editing disclosed herein.

Sill further, the cassette construction strategies are also used herein for optimization, as discussed herein after.

Specifically, the first strategy holds an advantage of expressing the CAR under the TRAC endogenous promoter and regulation, therefore preventing T cell exhaustion and reduced potency. The dependency of CAR expression under the TRAC locus also adds another level of specificity. The second and third strategies provide episomal CAR expression which could mitigate tumor progression, allowing more T cells to express the CAR at earlier time points. These two strategies include sophisticated designs, in which one transcript allows the translation of both the CAR/TCR and the ARCUS enzyme from the episomal AAV, bot only the CAR/TCR is expressed upon integration (because the ARCUS enzyme is coded outside of the homology arms). In each version a carefully planned combination of several elements was used to save space between the ITRs (splice acceptor, splice donor, 2A peptide, Furin cleavage site, GSG linkers, etc.). Of note, in version 2, the CAR gene is followed by a 2A peptide, positioned to allow a continuous reading frame with the downstream TRAC exons, in order to prevent nonsense mediated decay (NMD). In version 3, the CAR/TCR gene is preceded by a 2A peptide, to allow a continuous reading frame with the preceding VJ exon, to allow endogenous regulation by the V promoter upon integration.

Taken together, the present disclosure provides ‘all in one AAV’ engineering that clearly improve in vivo T cell transduction and killing potency, while reducing T cell exhaustion. This could lead to higher survival rates and reduced relapse rates of treated patients. The combination with capsid targeting can further increase potency and reduce off target transduction.

All scientific and technical terms used herein have meanings commonly used in the art unless otherwise specified. The definitions provided herein are to facilitate understanding of certain terms used frequently herein and are not meant to limit the scope of the present disclosure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. Thus, as used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, and/or parts, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

### Materials and Experimental Procedures

### Cell Lines and PBMCs

HEK-293 (ATCC CRL-1573), cells were grown in DMEM supplemented with 10% FCS and 1% glutamine. NALM6 cells were cultivated in DMEM supplemented with 10% FCS and 1% glutamine and 0.25 ug/ml G418.

### Human PBMCs Purification

PBMCs purified from a buffy coat were stimulated using 50 U/ml of interleukin-2 and 1 ug/ml of anti-CD28 (clone CD28.2) and anti-CD3 (clone OKT3) (Biogem) antibodies for 2-3 days in MEM alpha media supplemented with 10% Heat Inactivated FBS, 1% glutamine and transplanted by intravenous tail vein injection (10-15 million cells/mouse).

### Animals

6-10 weeks old NOD.Cg-Prkdcscid Il2rgtm1Wjl/SzJ (further on termed ‘NSG’) (The Jackson Laboratory, Bar Harbor, USA) male mice were housed and kept at ambient temperature of 19-23° C., humidity of 45-65% and with a 12 h light/12 h dark cycle.

### Mice Experiments

Mice were engrafted intravenously with 180-200k Luc-GFP NALM6 stably expressing cell line in 200 ul PBS. Following imaging of initial tumors, mice were injected by 1E12 vg/vector/200 μl/mouse in PBS intravenously with were engrafted intravenously with 10-15 million activated T cells. Indicated AAV vector preparations 5-6 days after engraftment. For in vivo imaging of the activity of the GL4-luciferase (Gold-BIO), mice were intra-peritoneally injected with 150 mg D-luciferin per kg body weight and anaesthetized using 0.1 mg/g and 0.001 mg/g Ketamine and Xylazine, respectively. Imaging data were obtained 10 min after substrate injection using a noninvasive cooled charged-coupled device (IVIS Lumina III Spectrum; Caliper Life Sciences). Data were analyzed using the Living Image Software (Caliper Life Sciences).

Blood samples from mice were collected in heparin. Cells and serum were separated by centrifugation. Serum was collected from the supernatant. For spleens, whole spleens were extracted from mice and mechanically crushed in PBS to be filtered in a 70 μm Cell Strainer (Corning). For bone marrow, cells were flushed from posterior femur and tibia. For blood, spleen and bone marrow, cells were processed with Red Blood Cell Lysis (Biolegend) and stained for T cell and B cells markers and CAR expression.

The planned use of animals in this research is compatible with the standards for care and use of laboratory animals and has been approved and assigned the number 04-19-045 of institutional care and use committee of Tel-Aviv university.

### Western Blotting

1E10 vector genomes containing the DARPIN-VP1 fusion protein and w.t AAVDJ vector were analyzed on a 12% SDS-polyacrylamide gel. Proteins blotted on a nitrocellulose membrane (Invitrogen) were detected using either the capsid protein-specific antibody B1 recognizing VP1, VP2 and VP3 because of their identical C-terminal region (Progen, Heidelberg, Germany) Signals were visualized by enhanced chemiluminescence using the ECL Plus Western Blotting Detection System (biological industries)

### Cloning

ARCUS (comprising the nucleic acid sequence as denoted by SEQ ID NO: 6) and human CD19-CAR (comprising the nucleic acid sequence as denoted by SEQ ID NO: 5) blocks were ordered from IDT and cloned into a single strand expression vector pAB270 (Barzel, A. et al. Promoterless gene targeting without nucleases ameliorates haemophilia B in mice. Nature 517, 360-364 (2015).) harboring ITR using PacI and MluI enzymes (NEB). Homology arms (LHA and RHA, that comprise the nucleic acid sequence as denoted by SEQ ID Nos: 9 and 10, respectively) were amplified from human T cell genome by PCR using PrimeStar Max (Takara) and assembled by Gibson assembly, Hi-Fi DNA Assembly Mix, (NEB).

Capsid engineering was made upon AAVDJ backbone by genetically fusing the anti-CD8 DARPIN into the GH2-GH3 surface loop of the VP1 capsid gene of AAV-DJ. In order to disrupt heparin sulfate binding, Arginines R587 and R590 of VP1, were further mutated to Alanine residues in the plasmid encoding the VP1-DARPIN fusion, as denoted by SEQ ID NO: 37). The splice acceptor (SA) is inactivated to prevent incorporation of DARPIN into the VP2 and VP3 capsid proteins. Expression of unmodified VP2 and VP3 is provided by a second plasmid, in which the start codon (Met) of VP1 was inactivated to prevent incorporation of unmodified VP1 in the capsid.

### AAV Production

rAAV-DJ_DARPIN CD8 were produced in 293T at 80% confluency cells with quadruplet transient transfection of VP1 CD8 DARPIN: VP2/VP3, ITR plasmid, AD5 helper plasmid (6.3 ug: 6.3 ug: 4.4 ug: 25 ug) for a total of 42 ug DNA per plate and a ratio of 1:2.5 Polyethylenimine (PEI) (Polysciences Inc) and a total of 60 plates per vector. AAV is harvested three days post transfection and purified by cesium chloride gradient in an ultra-centrifugation. Quantification of the particles is made by qPCR with suitable primers and Syber green enzyme.

Titer quantification was performed by qPCR using SYBRGreen (PCRBiosystems) by StepOne qPCR machine (thermofisher).

### In Vitro T Cell Activation and Engineering

Human PBMC are activated with a coated plate with 1 ug/ml human CD3 (clone OKT3) and CD28 (clone CD28.2) supplemented with human IL-2 50 U/ml in MEM-alpha media for 48-72 h. The cells were washed from activation media and were subsequently subjected to transduction with 500k viral genomes/cell in a 96 well plate for 1 h at 37 degrees. Continuously, media was added and engineering efficiency rate is measured by flow cytometry 3 days post transduction (CD3-CAR+) using CD3 antibody (clone okt3, Biolegend) and CD19 antigen (AcroBiosytems). Furtherer analysis include genomic PCR for CAR integration, reverse transcription for CAR/ARCUS expression, ELISA for IFN-gamma secretion (R&D systems), activation (CD25, CD69, CD107) and killing assays (CFSE and PI) followed by co-culturing or expansion of target or control cells with engineered cells and analysis by flow cytometry.

### Example 1

### TCR Alpha Constant (TRAC) Locus Targeting

For targeting exogenous nucleic acid sequences into the T cell receptor (TCR) alpha constant (TRAC) locus, adeno associated viral vectors (AAV) were used. As proof of concept, nucleic acid sequence encoding a CD19 chimeric antigen receptor (CAR), was used as an exogenous nucleic acid sequence of interest. “ARCUS”, an engineered derivative of the I-CreI homing endonuclease, was used for gene editing [Daniel T. MacLeod. Molecular Therapy Vol. 25 No 4; pages 949-961 (2017) [3]]. An AAV vector coding for the CAR as well as for a nuclease targeting the CAR into the TCR alpha constant (TRAC) locus, was designed. FIG. 1A (i)-(iii), illustrates various optional engineering strategies. More specifically, FIG. 1A(i) is a schematic presentation of the AAV construct, and targeted integration strategy. As shown by the figure, the anti-human CD19 CAR (hCD19 CAR) is preceded by a sequence coding for a Furin and 2A peptide (2A) and followed by a polyadenylation signal (PolyA). The CAR cassette, flanked by homology arms (LHA and RHA), is followed by a cassette coding for the ARCUS nuclease under the Jet promoter. The ARCUS nuclease is episomally expressed, leading to a double strain break (DSB) at the TRAC locus. A CAR cassette flanked by homology arms (HAs), is integrated at the target site and expressed under the TRAC endogenous promoter, FIGS. 1A(ii) and 1A(iii) demonstrate alternative strategies for engineering, which support higher CAR expression levels, due to the episomal expression of the CAR.

By that, the CAR cassette could be further expressed independently of ARCUS activity and enhance cytotoxic activity and tumor rejection. FIG. 1B, schematically represents the genetic fusion of the anti-CD8 DARPIN into the GH2-GH3 surface loop of the VP1 capsid gene of AAV-DJ. In order to disrupt heparin sulfate binding, Arginine residues R587 and R590 were further mutated to Alanine residues in the plasmid encoding the VP1-DARPIN fusion. The splice acceptor (SA) is inactivated to prevent incorporation of the DARPIN into the VP2 and VP3 capsid proteins. Expression of unmodified VP2 and VP3 is provided by a second plasmid, in which the start codon (Met) of VP1 was inactivated to prevent incorporation of unmodified VP1 in the capsid. FIG. 1C shows a Western Blot of AAV_CD8_DARPIN preps and demonstrates the successful construction of the AAV vector. More specifically, as shown by the Figure, the expected size of the edited VP1-DARPIN compared to the w.t. VP1 of unmodified AAV. As expected, AAV_DARPIN CD8 prep demonstrated the 3 proteins: vp1-DARPIN CD8 size 100.6 kda, vp2: 66.6 kda, vp3: 60.1 kda.

### Example 2

### Specificity of Human Cell In Vitro Transduction by a GFP Coding CD8_DARPIN Targeted AAV

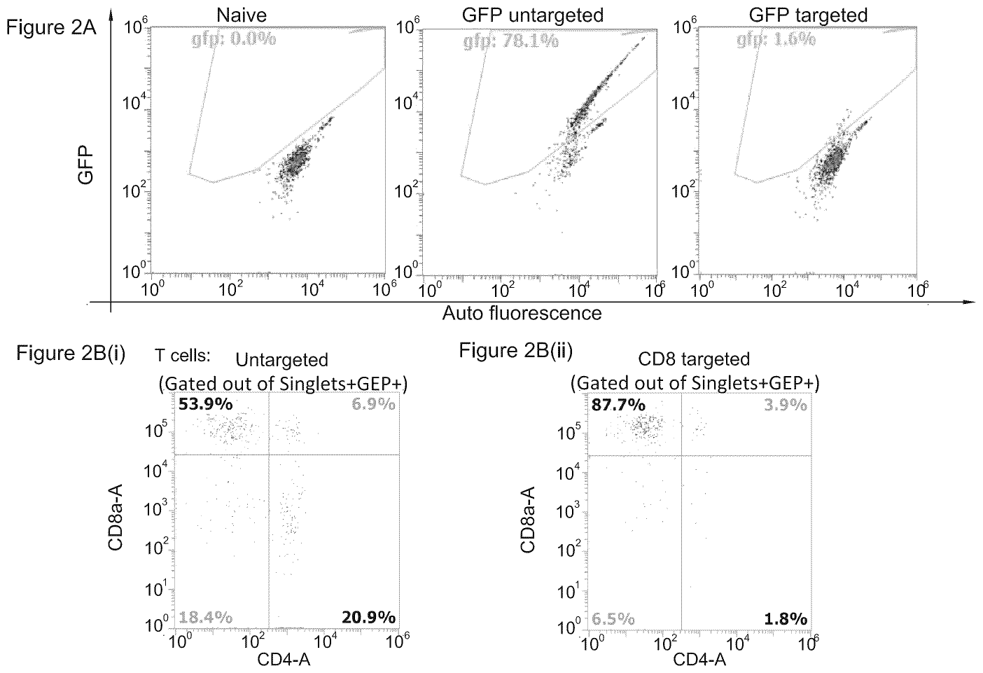

Transduction was initially performed using 293T cells. FIG. 2A shows 293T cells that were transduced with a GFP coding AAV-DJ vector with disrupted heparin sulfate binding and with an incorporated CD8_DARPIN at an MOI of 56K AAV genomes per cell. Fluorescence was measured 48 h later by flow cytometry. As demonstrated by the figure, disruption of the heparin sulfate resulted in almost complete abolishment of the transduction ability of AAV_DARPIN CD8 compared to the w.t vector. Next, Human primary T cells were transduced with a GFP coding AAV-DJ with an incorporated CD8_DARPIN (FIG. 2B(ii)) or untargeted AAV (FIG. 2B(i)), at an MOI of 56K AAV genomes per cell. The panel shows CD4+ and CD8+ cells gated out of (Singlets+GFP+). Florescence was measured 48 h later by flow cytometry. As shown, the targeted AAV lead to a transduction specificity towards CD8+ T cells.

### Example 3

### In Vitro Transduction of ARCUS-CAR AAV

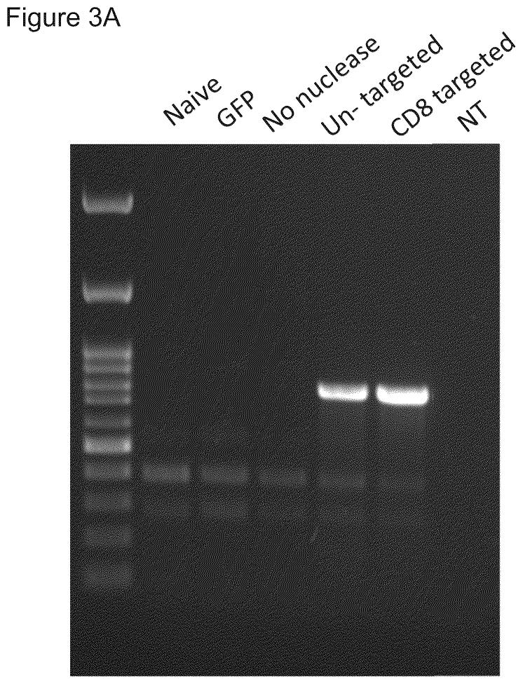

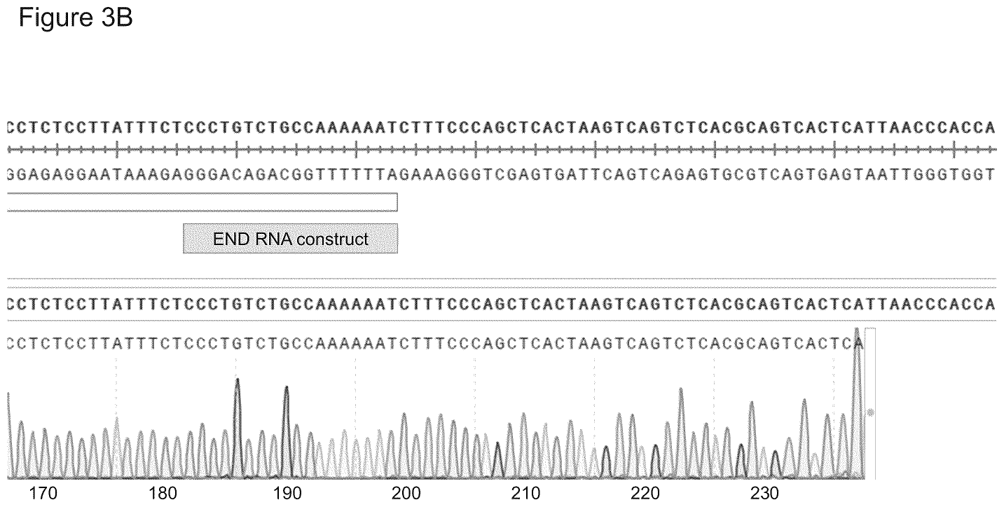

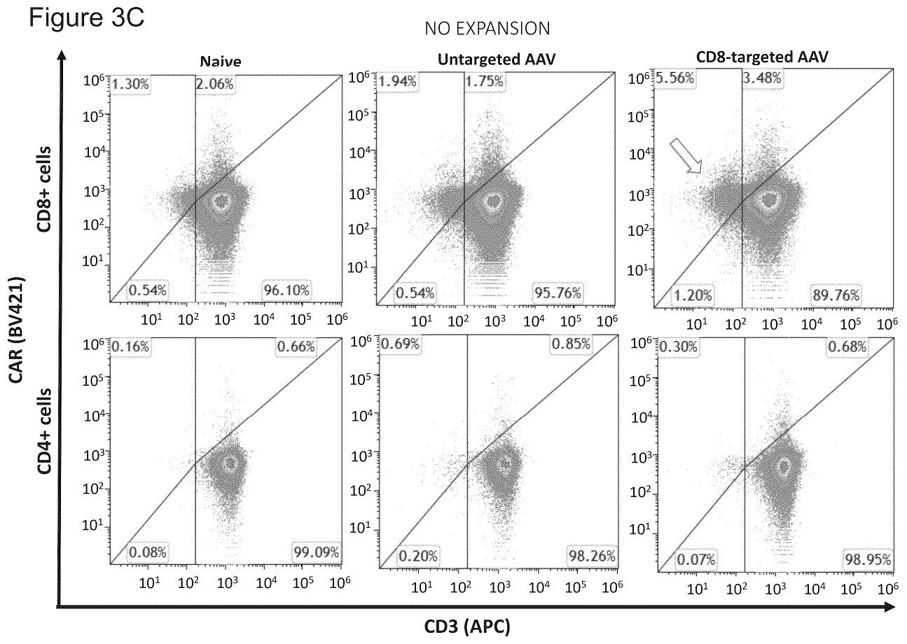

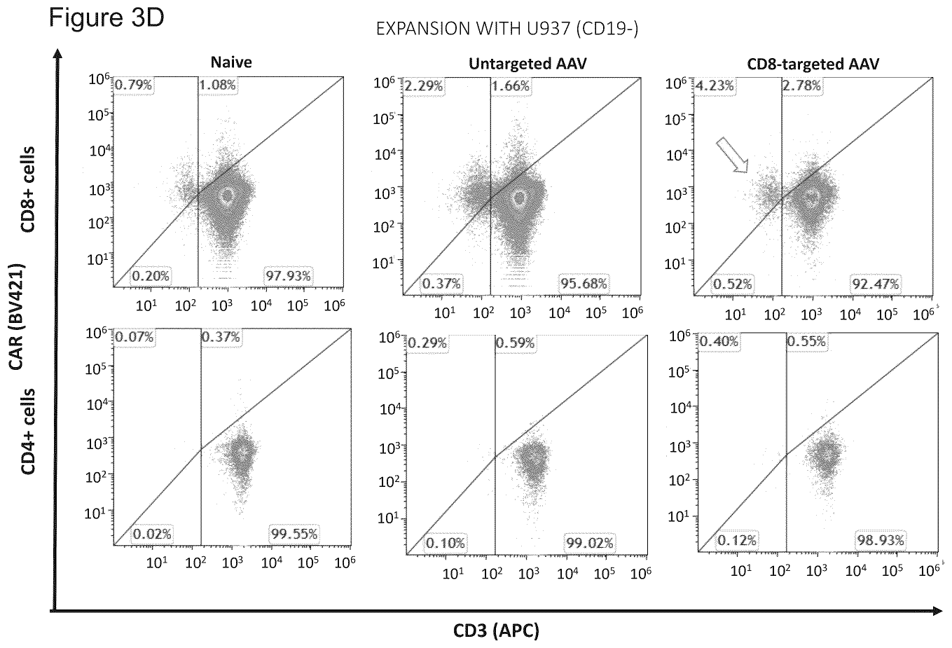

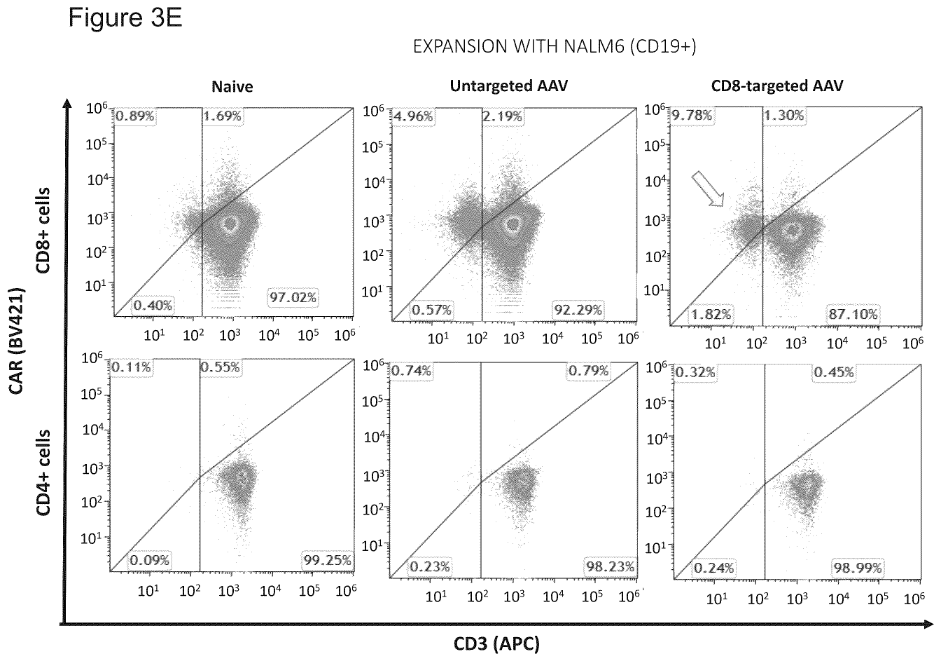

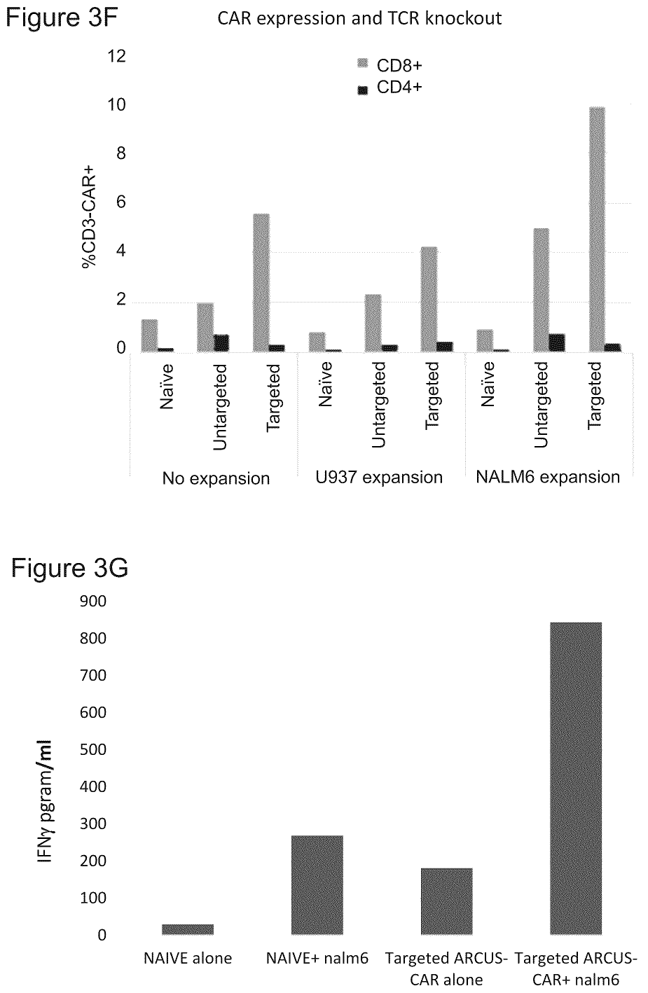

FIGS. 3A-3G show successful integration of the exogenous CAR into the TRAC locus with transduction of ARCUS-CAR AAV (MOI 500-750K) followed by CAR expression (2%-10%) under TRAC endogenous promoter. This design allows co-encapsulation of the CAR gene together with the nuclease gene in a single AAV to facilitate efficient in vivo T cell targeting. To control cell specific transduction of human T cells, DARPINs (designed ankyrin repeat proteins) which target the human CD8 receptor were used.

More specifically, FIG. 3A demonstrates successful genomic integration of the CAR into the TRAC locus. Genomic CAR integration is depicted in FIG. 3B by Sanger sequencing of the PCR product of ARCUS-CAR targeted AAV.

Next, cells were transduced with ARCUS-CAR coding AAV (targeted to CD8+ or untargeted, gated out of live, singlets, CD8+ or CD4+) and CAR expression was measured as shown by FIGS. 3C, 3D. and 3E.

As shown by the figure, CAR+CD3− cells were indeed expanded with NALM6 CD19+ compared to control, probably due to antigen binding, activation and proliferation.

Furthermore, as shown by FIG. 3G, IFN-gamma secretion was elevated when CAR T cells (but not un-transduced) were co-incubated with target cells NALM6 CD19⁺ compared to U937 CD19 minus cells.

### Example 4

### Site-Specific In Vivo T Cells Engineering to Express CAR

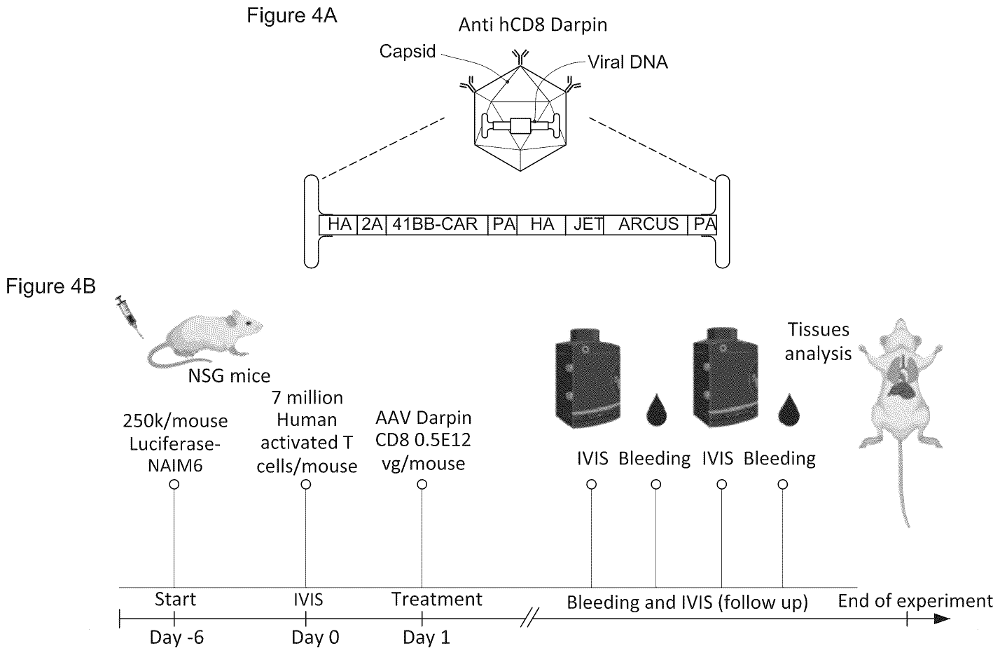

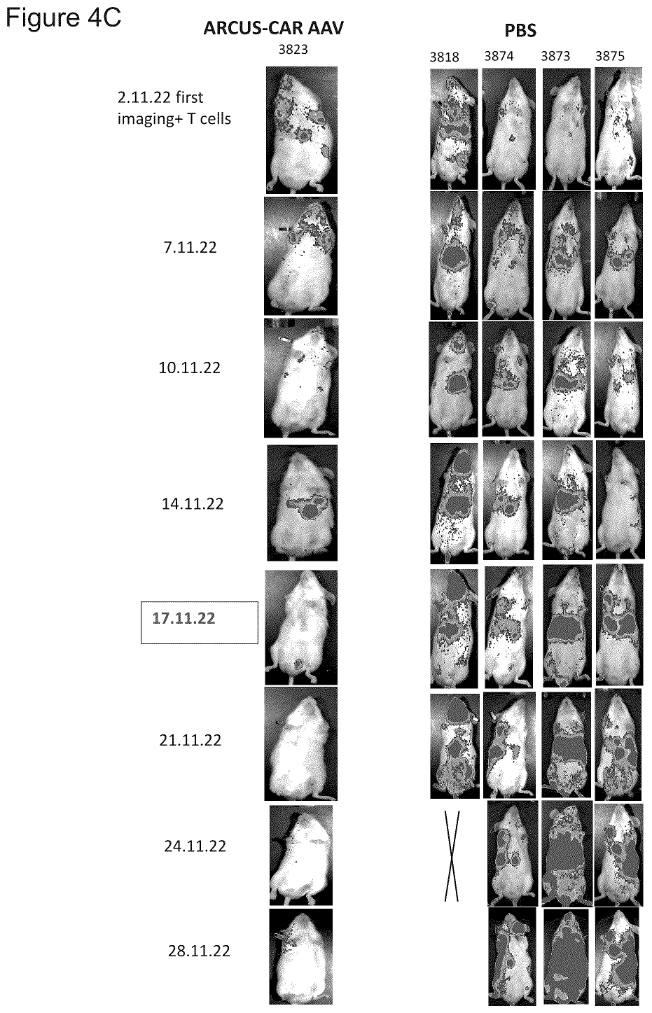

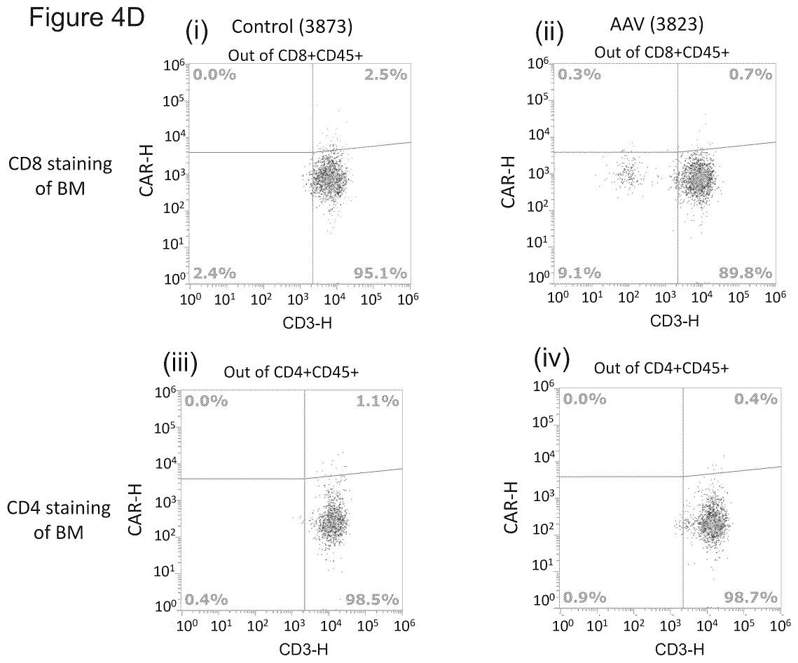

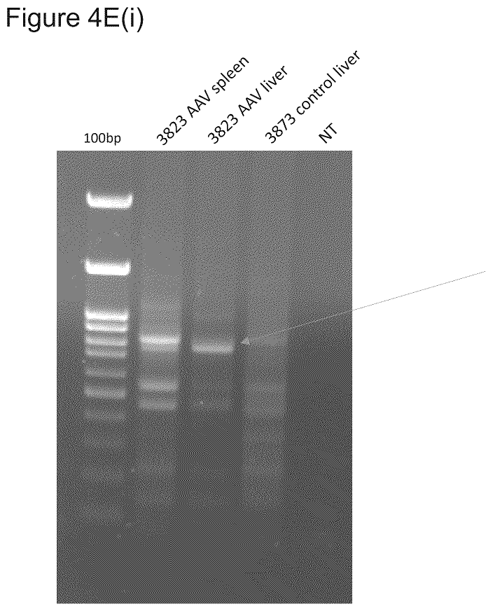

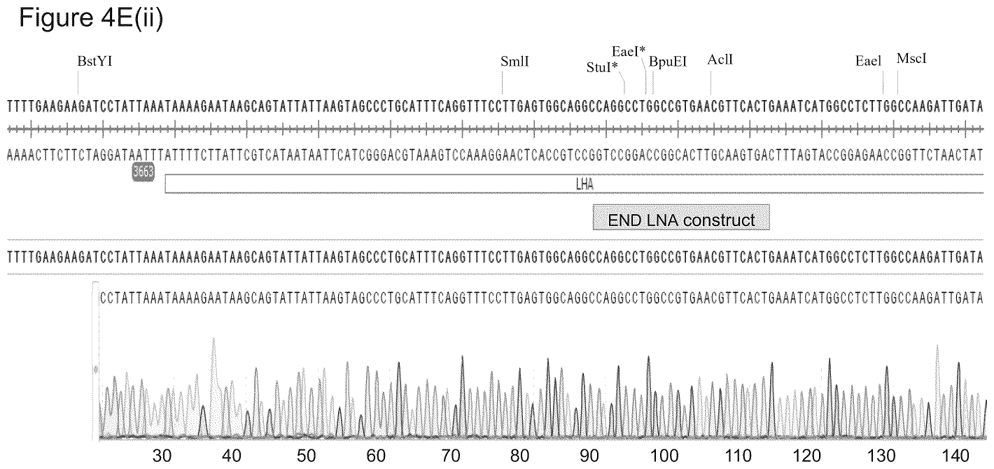

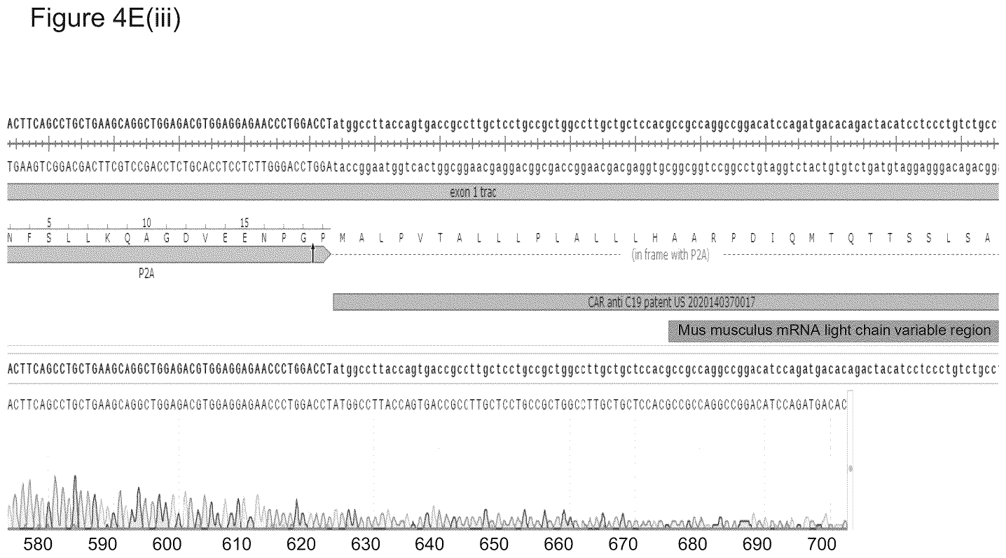

For in vivo transduction of ARCUS-CAR AAV (in vivo T cells engineering), human T cells are targeted in NSG mice that harbor leukemic B cells. CAR expression is led by episomal ARCUS expression, double strand break, and genomic CAR integration by homologous recombination. CAR expression is under the TRAC endogenous promoter led by 2A peptide for an ideal CAR expression. Anti-CD8 DARPIN is incorporated in the AAV capsid to target the AAV to CD8 T cells (FIG. 4A). Mice were injected with 250k NALM-LUC cells/mouse and 5 days later were injected with 7 million T cells/mouse. Subsequently, the mice received an injection of 0.5E12 vg/mouse ARCUS-CAR-DARPIN CD8 targeting AAV (FIG. 4B). Tumor size was measured routinely by IVIS. Treated AAV mouse number 3823, showed tumor reduction by IVIS (FIG. 4C). The mouse was sacrificed, and BM was extracted and stained for CD3. As shown, CD3 minus population is observed specifically only among the AAV-CD8-DARPIN treated mouse and only out of CD45+CD8+ cells, demonstrating specific T cell engineering and targeting by nuclease activity and CD8-DARPIN, respectively (FIG. 4D). Genomic CAR integration was shown in the liver of the AAV-CD8-DARPIN treated mouse and was verified by sequencing (FIG. 4E(i), and FIGS. 4E(ii), 4E(iii)). This data indicates an initial proof of concept for in vivo CAR T cell engineering.

1-58. (canceled)

59. A system for in vivo targeted insertion of at least one exogenous nucleic acid sequence of interest into a target genomic locus of a cell of the T lineage in a mammalian subject, said system comprising:
(a) at least one nucleic acid molecule comprising said at least one exogenous nucleic acid sequence of interest, said sequence is flanked on at least one of the 5′ and 3′ thereof by at least one homology arm, for integration to said target locus by homologous recombination, and/or at least one cassette/s and/or construct/s thereof, wherein said at least one nucleic acid molecule and/or at least one cassette/s and/or construct/s thereof, is comprised within at least one adeno associated virus (AAV) vector and/or AAV-like vector; and
(b) at least one site specific nuclease, or at least one nucleic acid sequence encoding said nuclease and/or at least one cassette/s and/or construct/s, and/or vector/s and/or delivery vehicle/s comprising said at least one nuclease or said nucleic acid sequence encoding said nuclease.

60. The system according to claim 59, wherein at least one of:
(a) said target locus is at least one of: T cell receptor (TCR) α chain locus, TCRδ chain locus, TCRγ chain locus and the TCRδ chain locus;
(b) said target locus is at least one of the TCR alpha constant (TRAC), and the TCR beta constant (TRBC) loci; and
(c) said target locus is the TRAC locus, and wherein said at least one homology arm enables the integration of said at least one exogenous nucleic acid sequence of interest into said TRAC.

61. The system according to claim 59, wherein at least one of:
(a) said at least one site specific nuclease is at least one of: at least one homing endonuclease, at least one zinc-finger nucleases (ZFNs), at least one transcription activator-like effector nuclease (TALEN), at least one clustered regulatory interspaced short palindromic repeat (CRISPR)/CRISPR associated (Cas) protein, and at least one Meganuclease-transcription activator-like (Mega-TAL);
(b) said homing nuclease is at least one member of the LAGLIDADG family of homing endonucleases; and
(c) said at least one member of the LAGLIDADG family of homing endonucleases is endonuclease I-CreI, or an engineered derivative thereof, optionally, said engineered derivative of endonuclease I-CreI is the ARCUS endonuclease that targets the TRAC locus.

62. The system according to claim 59, wherein said exogenous nucleic acid sequence of interest encodes at least one receptor molecule, optionally, said receptor molecule is at least one of: at least one chimeric antigen receptor (CAR) and/or at least one exogenous TCR, optionally, said nucleic acid sequence of interest encodes at least one CAR molecule.

63. The system according to claim 59, wherein at least one of:
(a) said at least one nucleic acid molecule comprising said at least one exogenous nucleic acid sequence of interest and said nucleic acid sequence encoding at least one site specific nuclease are comprised within the same cassette/s and/or construct/s, and/or vector/s, and wherein said vector is at least one AAV vector and/or AAV-like vector;
(b) said at least one nucleic acid molecule comprising said at least one exogenous nucleic acid sequence of interest and said nucleic acid sequence encoding at least one site specific nuclease are provided in separate cassette/s and/or construct/s, and/or vector/s and/or delivery vehicle/s, and wherein said at least one nucleic acid molecule and/or at least one cassette/s and/or construct/s thereof, is comprised within at least one AAV vector and/or AAV-like vector; and
(c) said cassette/s and/or construct/s, and/or vector/s and/or delivery vehicle/s further comprise at least one genetic element.

64. The system according to claim 59, wherein said at least one vector and/or delivery vehicle is any one of a viral vector, a non-viral vector and a naked DNA vector, optionally, said vector is a viral vector, said viral vector is any one of adeno associated virus (AAV) vector, AAV-like vector, recombinant adeno associated virus (rAAV), single stranded AAV (ssAAV), self-complementary rAAV (scAAV), Simian vacuolating virus 40 (SV40) vector, Adeno virus vector, helper-dependent Adeno viral vector, retroviral vector and lentiviral vector.

65. The system according to claim 59, wherein at least one of:
(a) said at least one vector and/or delivery vehicle further comprises at least one T cell targeting moiety; and
(b) said at least one exogenous nucleic acid sequence of interest further comprises an inducible suicide gene.

66. A viral vector for in vivo targeted insertion of at least one exogenous nucleic acid sequence of interest into a target genomic locus of a cell of the T lineage in a mammalian subject, said delivery vehicle comprising at least one of:
(a) at least one nucleic acid molecule comprising at least one exogenous nucleic acid sequence of interest, said sequence is flanked on at least one of the 5′ and 3′ thereof by at least one homology arm, for integration to said target locus by homologous recombination; and/or
(b) at least one site specific nuclease, or at least one nucleic acid sequence encoding said nuclease.

67. The viral vector according to claim 66, wherein at least one of:
(a) said viral vector is at least one AAV vector and/or AAV-like vector;
(b) said viral vector further comprises at least one targeting moiety; and
(c) viral vector comprises at least one targeting moiety incorporated in the capsid thereof, optionally, said at least one targeting moiety replaces at least one capsid protein of said viral vector or at least part thereof.

68. A method for in vivo targeted insertion of at least one exogenous nucleic acid sequence of interest into a target genomic locus of a cell of the T lineage, in a mammalian subject, said method comprising the step of administering to said subject an effective amount of:
(a) at least one nucleic acid molecule comprising said at least one exogenous nucleic acid sequence of interest, said sequence is flanked on at least one of the 5′ and 3′ thereof by at least one homology arm, for integration to said target locus by homologous recombination, and/or at least one cassette/s and/or construct/s thereof, wherein said at least one nucleic acid molecule and/or at least one cassette/s and/or construct/s thereof, is comprised within at least one AAV vector and/or AAV-like vector; and
(b) at least one site specific nuclease, or at least one nucleic acid sequence encoding said nuclease or any cassette, vector or vehicle comprising said at least one nuclease or said nucleic acid sequence encoding said nuclease; or
(c) any system, vector, delivery vehicle, matrix, nano- or micro-particle and/or composition comprising (a) and/or (b).

69. The method according to claim 68, wherein at least one of:
(a) said target locus is at least one of TCR α chain locus, TCRβ chain locus, TCRγ chain locus and the TCRδ chain locus;
(b) said target locus is at least one of the TRAC, and the TRBC loci; and
(c) said target locus is the TRAC locus, and wherein said at least one homology arm enables the integration of said at least one exogenous nucleic acid sequence of interest into said TRAC.

70. The method according to claim 68, wherein at least one of:
(a) said at least one site specific nuclease is at least one of: at least one homing endonuclease, at least one ZFN, at least one TALEN, at least one CRISPR/Cas protein, and at least one Mega-TAL;
(b) said homing nuclease is at least one member of the LAGLIDADG family of homing endonucleases; and
(c) said at least one member of the LAGLIDADG family of homing endonucleases is endonuclease I-CreI, or an engineered derivative thereof, optionally said engineered derivative of endonuclease I-CreI is the ARCUS endonuclease that targets the TRAC locus.

71. The method according to claim 68, wherein at least one of:
(a) said exogenous nucleic acid sequence of interest encodes at least one receptor molecule, optionally said receptor molecule is at least one of: at least one CAR and at least one exogenous TCR;
(b) said nucleic acid sequence of interest encodes at least one CAR molecule; and
(c) wherein said mammalian subject is a subject suffering from an immune-related disorder.

72. The method according to claim 68, wherein at least one of:
(a) said at least one nucleic acid molecule comprising said at least one exogenous nucleic acid sequence of interest and said at least one nucleic acid sequence encoding at least one site specific nuclease are comprised within the same cassette/s and/or construct/s, and/or vector/s, and wherein said vector is at least one AAV vector and/or AAV-like vector;
(b) said at least one nucleic acid molecule comprising said at least one exogenous nucleic acid sequence of interest and said at least one nucleic acid sequence encoding at least one site specific nuclease are provided in separate cassette/s and/or construct/s, and/or vector/s and/or delivery vehicle/s, and wherein said at least one nucleic acid molecule and/or at least one cassette/s and/or construct/s thereof, is comprised within at least one AAV vector and/or AAV-like vector;
(c) said cassette/s and/or construct/s, and/or vector/s and/or delivery vehicle/s further comprise at least one genetic element;
(d) wherein said vector is any one of a viral vector, a non-viral vector and a naked DNA vector, optionally, said viral vector is any one of AAV, AAV-like, rAAV, ssAAV, scAAV, SV40 vector, Adeno virus vector, helper-dependent Adeno viral vector, retroviral vector and lentiviral vector;
(e) said vector further comprises at least one T cell targeting moiety; and
(f) said at least one exogenous nucleic acid sequence of interest further comprises an inducible suicide gene.

73. The method according to claim 68, for treating, preventing, ameliorating, inhibiting or delaying the onset of an immune-related disorder in a mammalian subject, the method comprises the step of administering to said subject a therapeutically effective amount of:
(a) at least one nucleic acid molecule comprising at least one exogenous nucleic acid sequence of interest, said sequence is flanked on at least one of the 5′ and 3′ thereof by at least one homology arm, for integration by homologous recombination to at least one target locus of a cell of the T lineage in said subject, and/or at least one cassette/s and/or construct/s thereof, wherein said at least one nucleic acid molecule and/or at least one cassette/s and/or construct/s thereof, is comprised within at least one AAV vector and/or AAV-like vector; and
(b) at least one site specific nuclease, or at least one nucleic acid sequence encoding said nuclease and/or at least one cassette/s and/or construct/s, and/or vector/s and/or delivery vehicle/s comprising said at least one nuclease or said nucleic acid sequence encoding said nuclease; or
(c) any system, delivery vehicle, matrix, nano- or micro-particle and/or composition comprising (a) and/or (b).

74. The method according to claim 73, wherein at least one of:
(a) said target locus is at least one of TCR α chain locus, TCRβ chain locus, TCRγ chain locus and the TCRδ chain locus;
(b) said target locus is at least one of the TRAC, and the TRBC loci;
(c) said at least one site specific nuclease is at least one of: at least one homing endonuclease, at least one ZFN, at least one TALEN, at least one CRISPR/Cas protein, and at least one Mega-TAL;
(d) said homing nuclease is at least one member of the LAGLIDADG family of homing endonucleases, and wherein said at least one member of the LAGLIDADG family of homing endonucleases is endonuclease I-CreI, or an engineered derivative thereof, optionally, said engineered derivative of endonuclease I-CreI is the ARCUS endonuclease that targets the TRAC locus; and
(e) said exogenous nucleic acid sequence of interest encodes at least one of: at least one CAR and at least one exogenous TCR.

75. The method according to claim 73, wherein at least one of:
(a) said at least one nucleic acid molecule comprising said at least one exogenous nucleic acid sequence of interest and said nucleic acid sequence encoding at least one site specific nuclease are comprised within the same cassette/s and/or construct/s, and wherein said vector is at least one AAV vector and/or AAV-like vector;
(b) said at least one nucleic acid molecule comprising said at least one exogenous nucleic acid sequence of interest and said nucleic acid sequence encoding at least one site specific nuclease are provided in separate cassette/s and/or construct/s, and/or vector/s and/or delivery vehicle/s, and wherein said at least one nucleic acid molecule and/or at least one cassette/s and/or construct/s thereof, is comprised within at least one AAV vector and/or AAV-like vector; and
(c) said vector is any one of a viral vector, a non-viral vector and a naked DNA vector, optionally, said vector is a viral vector, said viral vector is any one of AAV, AAV-like, rAAV, ssAAV, scAAV, SV40 vector, Adeno virus vector, helper-dependent Adeno viral vector, retroviral vector and lentiviral vector.

76. The method according to claim 73, wherein said immune-related disorder comprise at least one of: a proliferative disorder, an inflammatory disorder, an infectious disease caused by a pathogen, an autoimmune disease, a neurodegenerative disease, a congenital disorder, an allergic condition, a cardiovascular disease, and a metabolic condition.

77. An in vivo genetically engineered cell of the T cell lineage, any population of cells comprising at least one said genetically modified cell, or any composition comprising said cell or population of cells, wherein said cell comprises at a modified TRAC and/or TRBC loci comprising at least one exogenous nucleic acid sequence of interest, and wherein said cell was genetically modified by at least one system is defined by claim 59.

78. A composition comprising an effective amount of at least one system or any cassette/s and/or construct/s, and/or vector/s and/or delivery vehicle/s, or any matrix, nano- or micro-particle thereof, for in vivo targeted insertion of at least one exogenous nucleic acid sequence of interest into a target genomic locus of a cell of the T lineage in a mammalian subject, wherein said system is as defined in claim 59, said composition optionally further comprises at least one pharmaceutically acceptable carrier/s, excipient/s, auxiliaries, and/or diluent/s.


SEQUENCE LISTING (52 sequences)

SEQ ID NO: 1 - protein, 9 aa, synthetic construct
  source 1..9: note = Meganucleases motif
LAGLIDADG

SEQ ID NO: 2 - DNA, 553 bp, synthetic construct
  source 1..553: mol_type = other DNA; note = ECMV IRES:
acgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataata

SEQ ID NO: 3 - DNA, 78 bp, synthetic construct
  source 1..78: mol_type = other DNA; note = Furin GSG P2A:
cgcgcgaaacgcggaagcggagctactaacttcagcctgctgaagcaggctggagacgtggaggagaaccctggacct

SEQ ID NO: 4 - protein, 500 aa, synthetic construct
  source 1..500: note = Anti-human CD19 CAR amino acids sequence: CD19 CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGRDYKDDDDKMHTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRY

SEQ ID NO: 5 - DNA, 1503 bp, synthetic construct
  source 1..1503: mol_type = other DNA; note = Anti-human CD19 CAR DNA sequence: CD19 CAR
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaagcggccgcgactacaaagacgatgacgacaagatgcataccaccacccctgcccctcgaccaccaactcctgctcctacaatagcttcacaacccctcagccttaggcccgaggcctgtaggcctgcagcagggggagctgtccacacccggggacttgatttcgcttgtgacatatatatctgggctcctctcgccggcacttgtggtgttcttctgctctctctcgtgatcacactctattgcaagcggggaaggaaaaaactgctctatatctttaagcaaccatttatgcggcccgtccaaacaacacaggaagaggatggctgttcttgtcgattccctgaggaggaggaaggcgggtgtgaattgagggttaagttcagtcgaagtgctgacgccccagcatataaacagggccagaaccaactctataatgagctcaatctcggacgccgagaagaatacgacgtactcgataagcgacgaggcagagaccctgaaatgggagggaagccacgacgcaaaaacccccaagagggactttacaacgagcttcagaaagacaagatggctgaagcttactccgagattggcatgaaaggggaacggcgcagaggcaagggtcacgacgggctctatcaggggctttccactgccacaaaggacacctatgatgcactgcacatgcaggctctgccaccacgatattga

SEQ ID NO: 6 - DNA, 1061 bp, synthetic construct
  source 1..1061: mol_type = other DNA; note = ARCUS DNA sequence:
acaccaagtacaacaaagagttcctgctgtacctggccggcttcgttgatggcgacggcagcatcttcgccagcatctaccctcaccagcgggccaagttcaagcacttcctgaagctgaccttcgccgtgtaccagaaaacccagcggagatggttcctggacaagctggtggatgagatcggcgtgggctacgtgtacgatagcggcagcgtgtccgagtacagactgagcgagatcaagcccctgcacaactttctgacacagctccagccctttctgaaactgaagcagaaacaggccaacctggtgctgaagatcatcgagcaacttccttctgcaaaagaaagccccgataagttcctggaagtgtgcacctgggtcgaccagatcgccgctctgaacgacagcagaaccagaaagaccaccagcgagacagtgcgggccgtgcttgattctctgcctggctctgttggcggcctgagtccaagccaggcttcttctgctgccagcagcgcctcttcttctcctggatctggaatcagcgaggccctgagagctggtgctggttctggcaccggctataacaaagaatttctgctctatctcgccggatttgtggacggcgacggctccatctatgcctgcattgccccacgacagggcagcaagtttaagcacagactgaagctgggctttgccgtgggacagaaaactcagaggcggtggtttctcgataagctggtcgacgaaattggagtcggctatgtgtatgaccggggctccgtgtctgaatacgtgctgtccgagattaagccgctccataacttcctgacccagctgcagccattcctcaagctgaaacaaaagcaggccaatctcgtcctcaagattattgaacagctgccctctgccaaagaatcccctgacaaatttctcgaagtctgtacgtgggttgaccaaatcgctgccctcaatgactccaagacacgcaagaccacctccgaaaccgtcagagccgttctggatagcctgtctgagaagaaaaagagcagcccctga

SEQ ID NO: 7 - protein, 353 aa, synthetic construct
  source 1..353: note = ARCUS
NTKYNKEFLLYLAGFVDGDGSIFASIYPHQRAKFKHFLKLTFAVYQKTQRRWFLDKLVDEIGVGYVYDSGSVSEYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIAPRQGSKFKHRLKLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 8 - DNA, 22 bp, synthetic construct
  source 1..22: mol_type = other DNA; note = ARCUS recognition sequence:
tggcctggagcaacaaatctga

SEQ ID NO: 9 - DNA, 453 bp, synthetic construct
  source 1..453: mol_type = other DNA; note = Left homology arm sequence:
gcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaagca

SEQ ID NO: 10 - DNA, 450 bp, synthetic construct
  source 1..450: mol_type = other DNA; note = Right homology arm sequence:
gcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaat

SEQ ID NO: 11 - DNA, 195 bp, synthetic construct
  source 1..195: mol_type = other DNA; note = The JeT promoter:
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgaca

SEQ ID NO: 12 - DNA, 500 bp, synthetic construct
  source 1..500: mol_type = other DNA; note = PGK PROMOTER:
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 13 - DNA, 492 bp, synthetic construct
  source 1..492: mol_type = other DNA; note = SFFV PROMOTER:
ccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 14 - DNA, 757 bp, synthetic construct
  source 1..757: mol_type = other DNA; note = CMV PROMOTER:
gatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatc

SEQ ID NO: 15 - DNA, 1334 bp, synthetic construct
  source 1..1334: mol_type = other DNA; note = EF1A PROMOTER
agtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 16 - DNA, 440 bp, synthetic construct
  source 1..440: mol_type = other DNA; note = CD3 PROMOTER:
ctgatcagaaacaagaggatctgctaatgtggcttgtgtaatagtttctctcagtggatctaaagaggtggcacttccttgtttgtgagtaaatgtagttgtattgttcataggaagcccaaaatcttctttgtttgttaccagtgactattgctatttgttttttgttaccaggaaaggctattctaaattcccttacacttcctagaattctgaacaaactttcctcatagtctctgctttgtatcaatcatcccaaaatctttgcttcagaaagagcagtttgcacctgtttgtgttttaccccacacctagcccactgttgctttgacagtcttacaccatcaaaggcagaaggtagagaggcagatttcttctagttcctcccccactcttcctttttctgaatacccatgagtctgttggcggaggatagtgct

SEQ ID NO: 17 - DNA, 225 bp, synthetic construct
  source 1..225: mol_type = other DNA; note = BGH POLY A:
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 18 - DNA, 122 bp, synthetic construct
  source 1..122: mol_type = other DNA; note = SV40 POLY A:
aacttgtttattgcagcttataatggttacaaataaagcaatagcatcacaaatttcacaaataaagcatttttttcactgcattctagttgtggtttgtccaaactcatcaatgtatctta

SEQ ID NO: 19 - DNA, 8098 bp, synthetic construct
  source 1..8098: mol_type = other DNA; note = Strategy I, Whole construct DNA sequence (including backbone):
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtaaagatctgatatcatcgatcgcgatgcattaattaataagcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaagcaccgcgcgaaacgcggaagcggagctactaacttcagcctgctgaagcaggctggagacgtggaggagaaccctggacctatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccaccacccctgcccctcgaccaccaactcctgctcctacaatagcttcacaacccctcagccttaggcccgaggcctgtaggcctgcagcagggggagctgtccacacccggggacttgatttcgcttgtgacatatatatctgggctcctctcgccggtacttgtggtgttcttctgctctctctcgtgatcacactctattgcaagcggggaaggaaaaaactgctctatatctttaagcaaccatttatgcggcccgtccaaacaacacaggaagaggatggctgttcttgtcgattccctgaggaggaggaaggtgggtgtgaattgagggttaagttcagtcgaagtgctgacgccccagcatataaacagggtcagaaccaactctataatgagctcaatctcggacgccgagaagaatacgacgtactcgataagcgacgaggcagagaccctgaaatgggagggaagccacgacgcaaaaacccccaagagggactttacaacgagcttcagaaagacaagatggctgaagcttactccgagattggcatgaaaggggaacggcgcagaggtaagggtcacgacgggctctatcaggggctttccactgccacaaaggacacctatgatgcactgcacatgcaggctctgccaccacgatattgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatgagctcccgcgggggcccgacgcgtaaaaatgagctcccgctccgctagcgctaccggaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacaggccaccatggatccaaaaaagaagagaaaggttgatcccaagaaaaagaggaaagtggatcctaaaaaaaagcggaaagtaaacaccaagtacaacaaagagttcctgctgtacctggccggcttcgttgatggcgacggcagcatcttcgccagcatctaccctcaccagcgggccaagttcaagcacttcctgaagctgaccttcgccgtgtaccagaaaacccagcggagatggttcctggacaagctggtggatgagatcggcgtgggctacgtgtacgatagcggcagcgtgtccgagtacagactgagcgagatcaagcccctgcacaactttctgacacagctccagccctttctgaaactgaagcagaaacaggccaacctggtgctgaagatcatcgagcaacttccttctgcaaaagaaagccccgataagttcctggaagtgtgcacctgggtcgaccagatcgccgctctgaacgacagcagaaccagaaagaccaccagcgagacagtgcgggccgtgcttgattctctgcctggctctgttggcggcctgagtccaagccaggcttcttctgctgccagcagcgcctcttcttctcctggatctggaatcagcgaggccctgagagctggtgctggttctggcaccggctataacaaagaatttctgctctatctcgccggatttgtggacggcgacggctccatctatgcctgcattgccccacgacagggcagcaagtttaagcacagactgaagctgggctttgccgtgggacagaaaactcagaggcggtggtttctcgataagctggtcgacgaaattggagtcggctatgtgtatgaccggggctccgtgtctgaatacgtgctgtccgagattaagccgctccataacttcctgacccagctgcagccattcctcaagctgaaacaaaagcaggccaatctcgtcctcaagattattgaacagctgccctctgccaaagaatcccctgacaaatttctcgaagtctgtacgtgggttgaccaaatcgctgccctcaatgactccaagacacgcaagaccacctccgaaaccgtcagagccgttctggatagcctgtctgagaagaaaaagagcagcccctgacccgggaccgataagcttagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtggtaccactagtacgcgtgatatcagatctgttacgtagataagtagcatggcgggttaatcattaactacaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaagcgcgcagctgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgcttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggcagctgcgcgctcgaacttcatgcctgccgaccttccccaggtcacgatccggacggcgggtgagttcacattttarcagccggacgtgcaractccgctggtggtctaacgtcggttaggtcccttgaatcacgggacatatgttggtgttggaggt

SEQ ID NO: 20 - DNA, 4469 bp, synthetic construct
  source 1..4469: mol_type = other DNA; note = Strategy I, cassette sequence (between ITRs):
ggaggggtggagtcgtgacgtaaagatctgatatcatcgatcgcgatgcattaattaataagcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaagcaccgcgcgaaacgcggaagcggagctactaacttcagcctgctgaagcaggctggagacgtggaggagaaccctggacctatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccaccacccctgcccctcgaccaccaactcctgctcctacaatagcttcacaacccctcagccttaggcccgaggcctgtaggcctgcagcagggggagctgtccacacccggggacttgatttcgcttgtgacatatatatctgggctcctctcgccggtacttgtggtgttcttctgctctctctcgtgatcacactctattgcaagcggggaaggaaaaaactgctctatatctttaagcaaccatttatgcggcccgtccaaacaacacaggaagaggatggctgttcttgtcgattccctgaggaggaggaaggtgggtgtgaattgagggttaagttcagtcgaagtgctgacgccccagcatataaacagggtcagaaccaactctataatgagctcaatctcggacgccgagaagaatacgacgtactcgataagcgacgaggcagagaccctgaaatgggagggaagccacgacgcaaaaacccccaagagggactttacaacgagcttcagaaagacaagatggctgaagcttactccgagattggcatgaaaggggaacggcgcagaggtaagggtcacgacgggctctatcaggggctttccactgccacaaaggacacctatgatgcactgcacatgcaggctctgccaccacgatattgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatgagctcccgcgggggcccgacgcgtaaaaatgagctcccgctccgctagcgctaccggaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacaggccaccatggatccaaaaaagaagagaaaggttgatcccaagaaaaagaggaaagtggatcctaaaaaaaagcggaaagtaaacaccaagtacaacaaagagttcctgctgtacctggccggcttcgttgatggcgacggcagcatcttcgccagcatctaccctcaccagcgggccaagttcaagcacttcctgaagctgaccttcgccgtgtaccagaaaacccagcggagatggttcctggacaagctggtggatgagatcggcgtgggctacgtgtacgatagcggcagcgtgtccgagtacagactgagcgagatcaagcccctgcacaactttctgacacagctccagccctttctgaaactgaagcagaaacaggccaacctggtgctgaagatcatcgagcaacttccttctgcaaaagaaagccccgataagttcctggaagtgtgcacctgggtcgaccagatcgccgctctgaacgacagcagaaccagaaagaccaccagcgagacagtgcgggccgtgcttgattctctgcctggctctgttggcggcctgagtccaagccaggcttcttctgctgccagcagcgcctcttcttctcctggatctggaatcagcgaggccctgagagctggtgctggttctggcaccggctataacaaagaatttctgctctatctcgccggatttgtggacggcgacggctccatctatgcctgcattgccccacgacagggcagcaagtttaagcacagactgaagctgggctttgccgtgggacagaaaactcagaggcggtggtttctcgataagctggtcgacgaaattggagtcggctatgtgtatgaccggggctccgtgtctgaatacgtgctgtccgagattaagccgctccataacttcctgacccagctgcagccattcctcaagctgaaacaaaagcaggccaatctcgtcctcaagattattgaacagctgccctctgccaaagaatcccctgacaaatttctcgaagtctgtacgtgggttgaccaaatcgctgccctcaatgactccaagacacgcaagaccacctccgaaaccgtcagagccgttctggatagcctgtctgagaagaaaaagagcagcccctgacccgggaccgataagcttagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtggtaccactagtacgcgtgatatcagatctgttacgtagataagtagcatggcgggttaatcattaactaca

SEQ ID NO: 21 - DNA, 3399 bp, synthetic construct
  source 1..3399: mol_type = other DNA; note = Strategy I, integrated cassette to target site in exon 1 of TRAC:
aaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaagcaccgcgcgaaacgcggaagcggagctactaacttcagcctgctgaagcaggctggagacgtggaggagaaccctggacctatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaagcggccgcgactacaaagacgatgacgacaagatgcataccaccacccctgcccctcgaccaccaactcctgctcctacaatagcttcacaacccctcagccttaggcccgaggcctgtaggcctgcagcagggggagctgtccacacccggggacttgatttcgcttgtgacatatatatctgggctcctctcgccggtacttgtggtgttcttctgctctctctcgtgatcacactctattgcaagcggggaaggaaaaaactgctctatatctttaagcaaccatttatgcggcccgtccaaacaacacaggaagaggatggctgttcttgtcgattccctgaggaggaggaaggtgggtgtgaattgagggttaagttcagtcgaagtgctgacgccccagcatataaacagggtcagaaccaactctataatgagctcaatctcggacgccgagaagaatacgacgtactcgataagcgacgaggcagagaccctgaaatgggagggaagccacgacgcaaaaacccccaagagggactttacaacgagcttcagaaagacaagatggctgaagcttactccgagattggcatgaaaggggaacggcgcagaggtaagggtcacgacgggctctatcaggggctttccactgccacaaaggacacctatgatgcactgcacatgcaggctctgccaccacgatattgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggacccta

SEQ ID NO: 22 - DNA, 4281 bp, synthetic construct
  source 1..4281: mol_type = other DNA; note = Strategy II, cassette sequence (between ITRs):
ggaggggtggagtcgtgacgtaaagatctgatatcatcgatcgcgatgcattaattaagcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaaaacttgtttattgcagcttataatggttacaaataaagcaatagcatcacaaatttcacaaataaagcatttttttcactgcattctagttgtggtttgtccaaactcatcaatgtatcttatccgctagcgctaccggaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacaggccaccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggcaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccaccacccctgcccctcgaccaccaactcctgctcctacaatagcttcacaacccctcagccttaggcccgaggcctgtaggcctgcagcagggggagctgtccacacccggggacttgatttcgcttgtgacatatatatctgggctcctctcgccggcacttgtggtgttcttctgctctctctcgtgatcacactctattgcaagcggggaaggaaaaaactgctctatatctttaagcaaccatttatgcggcccgtccaaacaacacaggaagaggatggctgttcttgtcgattccctgaggaggaggaaggcgggtgtgaattgagggttaagttcagtcgaagtgctgacgccccagcatataaacagggccagaaccaactctataatgagctcaatctcggacgccgagaagaatacgacgtactcgataagcgacgaggcagagaccctgaaatgggagggaagccacgacgcaaaaacccccaagagggactttacaacgagcttcagaaagacaagatggctgaagcttactccgagattggcatgaaaggggaacggcgcagaggcaagggtcacgacgggctctatcaggggctttccactgccacaaaggacacctatgatgcactgcacatgcaggctctgccaccacgatatcgcgcgaaacgcggaagcggagctactaacttcagcctgctgaagcaggctggagacgtggaggagaaccctggacctagcccaggtaagggcgcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatcctgatcctcttgtcccacagatatccagaaccctgaccctgccaccatggatccaaaaaagaagagaaaggttgatcccaagaaaaagaggaaagtggatcctaaaaaaaagcggaaagtaaacaccaagtacaacaaagagttcctgctgtacctggccggcttcgttgatggcgacggcagcatcttcgccagcatctaccctcaccagcgggccaagttcaagcacttcctgaagctgaccttcgccgtgtaccagaaaacccagcggagatggttcctggacaagctggtggatgagatcggcgtgggctacgtgtacgatagcggcagcgtgtccgagtacagactgagcgagatcaagcccctgcacaactttctgacacagctccagccctttctgaaactgaagcagaaacaggccaacctggtgctgaagatcatcgagcaacttccttctgcaaaagaaagccccgataagttcctggaagtgtgcacctgggtcgaccagatcgccgctctgaacgacagcagaaccagaaagaccaccagcgagacagtgcgggccgtgcttgattctctgcctggctctgttggcggcctgagtccaagccaggcttcttctgctgccagcagcgcctcttcttctcctggatctggaatcagcgaggccctgagagctggtgctggttctggcaccggctataacaaagaatttctgctctatctcgccggatttgtggacggcgacggctccatctatgcctgcattgccccacgacagggcagcaagtttaagcacagactgaagctgggctttgccgtgggacagaaaactcagaggcggtggtttctcgataagctggtcgacgaaattggagtcggctatgtgtatgaccggggctccgtgtctgaatacgtgctgtccgagattaagccgctccataacttcctgacccagctgcagccattcctcaagctgaaacaaaagcaggccaatctcgtcctcaagattattgaacagctgccctctgccaaagaatcccctgacaaatttctcgaagtctgtacgtgggttgaccaaatcgctgccctcaatgactccaagacacgcaagaccacctccgaaaccgtcagagccgttctggatagcctgtctgagaagaaaaagagcagcccctgacccgggaccgataagcttaacttgtttattgcagcttataatggttacaaataaagcaatagcatcacaaatttcacaaataaagcatttttttcactgcattctagttgtggtttgtccaaactcatcaatgtatcttaacgcgtgatatcagatctgttacgtagataagtagcatggcgggtacgcgtgatatcagatctgttacgtagataagtagcatggcgggttaatcattaactaca

SEQ ID NO: 23 - DNA, 3239 bp, synthetic construct
  source 1..3239: mol_type = other DNA; note = Strategy II, integrated cassette to target site in exon 1 of TRAC:
acctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaaaacttgtttattgcagcttataatggttacaaataaagcaatagcatcacaaatttcacaaataaagcatttttttcactgcattctagttgtggtttgtccaaactcatcaatgtatcttatccgctagcgctaccggaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacaggccaccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggcaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaagcggccgcgactacaaagacgatgacgacaagatgcataccaccacccctgcccctcgaccaccaactcctgctcctacaatagcttcacaacccctcagccttaggcccgaggcctgtaggcctgcagcagggggagctgtccacacccggggacttgatttcgcttgtgacatatatatctgggctcctctcgccggcacttgtggtgttcttctgctctctctcgtgatcacactctattgcaagcggggaaggaaaaaactgctctatatctttaagcaaccatttatgcggcccgtccaaacaacacaggaagaggatggctgttcttgtcgattccctgaggaggaggaaggcgggtgtgaattgagggttaagttcagtcgaagtgctgacgccccagcatataaacagggccagaaccaactctataatgagctcaatctcggacgccgagaagaatacgacgtactcgataagcgacgaggcagagaccctgaaatgggagggaagccacgacgcaaaaacccccaagagggactttacaacgagcttcagaaagacaagatggctgaagcttactccgagattggcatgaaaggggaacggcgcagaggcaagggtcacgacgggctctatcaggggctttccactgccacaaaggacacctatgatgcactgcacatgcaggctctgccaccacgatatcgcgcgaaacgcggaagcggagctactaacttcagcctgctgaagcaggctggagacgtggaggagaaccctggacctagcccaggtaagggcgcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagatt

SEQ ID NO: 24 - DNA, 4107 bp, synthetic construct
  source 1..4107: mol_type = other DNA; note = Strategy III, cassette sequence (between ITRs):
ggaggggtggagtcgtgacgtaaagatctgatatcatcgatcgcgatgcattaattaatccgctagcgctaccggaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacaggccaccatggatccaaaaaagaagagaaaggttgatcccaagaaaaagaggaaagtggatcctaaaaaaaagcggaaagtaaacaccaagtacaacaaagagttcctgctgtacctggccggcttcgttgatggcgacggcagcatcttcgccagcatctaccctcaccagcgggccaagttcaagcacttcctgaagctgaccttcgccgtgtaccagaaaacccagcggagatggttcctggacaagctggtggatgagatcggcgtgggctacgtgtacgatagcggcagcgtgtccgagtacagactgagcgagatcaagcccctgcacaactttctgacacagctccagccctttctgaaactgaagcagaaacaggccaacctggtgctgaagatcatcgagcaacttccttctgcaaaagaaagccccgataagttcctggaagtgtgcacctgggtcgaccagatcgccgctctgaacgacagcagaaccagaaagaccaccagcgagacagtgcgggccgtgcttgattctctgcctggctctgttggcggcctgagtccaagccaggcttcttctgctgccagcagcgcctcttcttctcctggatctggaatcagcgaggccctgagagctggtgctggttctggcaccggctataacaaagaatttctgctctatctcgccggatttgtggacggcgacggctccatctatgcctgcattgccccacgacagggcagcaagtttaagcacagactgaagctgggctttgccgtgggacagaaaactcagaggcggtggtttctcgataagctggtcgacgaaattggagtcggctatgtgtatgaccggggctccgtgtctgaatacgtgctgtccgagattaagccgctccataacttcctgacccagctgcagccattcctcaagctgaaacaaaagcaggccaatctcgtcctcaagattattgaacagctgccctctgccaaagaatcccctgacaaatttctcgaagtctgtacgtgggttgaccaaatcgctgccctcaatgactccaagacacgcaagaccacctccgaaaccgtcagagccgttctggatagcctgtctgagaagaaaaagagcagcccccgcgcgaaacgcggaagcggaagcccaggtaagggcgcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaaagctactaacttcagcctgctgaagcaggctggagacgtggaggagaaccctggacctatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaagcggccgcgactacaaagacgatgacgacaagatgcataccaccacccctgcccctcgaccaccaactcctgctcctacaatagcttcacaacccctcagccttaggcccgaggcctgtaggcctgcagcagggggagctgtccacacccggggacttgatttcgcttgtgacatatatatctgggctcctctcgccggcacttgtggtgttcttctgctctctctcgtgatcacactctattgcaagcggggaaggaaaaaactgctctatatctttaagcaaccatttatgcggcccgtccaaacaacacaggaagaggatggctgttcttgtcgattccctgaggaggaggaaggcgggtgtgaattgagggttaagttcagtcgaagtgctgacgccccagcatataaacagggccagaaccaactctataatgagctcaatctcggacgccgagaagaatacgacgtactcgataagcgacgaggcagagaccctgaaatgggagggaagccacgacgcaaaaacccccaagagggactttacaacgagcttcagaaagacaagatggctgaagcttactccgagattggcatgaaaggggaacggcgcagaggcaagggtcacgacgggctctatcaggggctttccactgccacaaaggacacctatgatgcactgcacatgcaggctctgccaccacgatattgacccgggaccgataagcttaacttgtttattgcagcttataatggttacaaataaagcaatagcatcacaaatttcacaaataaagcatttttttcactgcattctagttgtggtttgtccaaactcatcaatgtatcttagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatacgcgtgatatcagatctgttacgtagataagtagcatggcgggttaatcattaactaca

SEQ ID NO: 25 - DNA, 3079 bp, synthetic construct
  source 1..3079: mol_type = other DNA; note = Strategy III, integrated cassette to target site in exon 1 of TRAC:
acctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaaagctactaacttcagcctgctgaagcaggctggagacgtggaggagaaccctggacctatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggcaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaagcggccgcgactacaaagacgatgacgacaagatgcataccaccacccctgcccctcgaccaccaactcctgctcctacaatagcttcacaacccctcagccttaggcccgaggcctgtaggcctgcagcagggggagctgtccacacccggggacttgatttcgcttgtgacatatatatctgggctcctctcgccggcacttgtggtgttcttctgctctctctcgtgatcacactctattgcaagcggggaaggaaaaaactgctctatatctttaagcaaccatttatgcggcccgtccaaacaacacaggaagaggatggctgttcttgtcgattccctgaggaggaggaaggcgggtgtgaattgagggttaagttcagtcgaagtgctgacgccccagcatataaacagggccagaaccaactctataatgagctcaatctcggacgccgagaagaatacgacgtactcgataagcgacgaggcagagaccctgaaatgggagggaagccacgacgcaaaaacccccaagagggactttacaacgagcttcagaaagacaagatggctgaagcttactccgagattggcatgaaaggggaacggcgcagaggcaagggtcacgacgggctctatcaggggctttccactgccacaaaggacacctatgatgcactgcacatgcaggctctgccaccacgatattgacccgggaccgataagcttaacttgtttattgcagcttataatggttacaaataaagcaatagcatcacaaatttcacaaataaagcatttttttcactgcattctagttgtggtttgtccaaactcatcaatgtatcttagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaag

SEQ ID NO: 26 - DNA, 21 bp, synthetic construct
  source 1..21: mol_type = other DNA; note = Furin GSG
cgcgcgaaacgcggaagcgga

SEQ ID NO: 27 - DNA, 57 bp, synthetic construct
  source 1..57: mol_type = other DNA; note = P2A
gctactaacttcagcctgctgaagcaggctggagacgtggaggagaaccctggacct

SEQ ID NO: 28 - protein, 7 aa, synthetic construct
  source 1..7: note = Furin GSG
RAKRGSG

SEQ ID NO: 29 - protein, 19 aa, synthetic construct
  source 1..19: note = p2A amino acids sequence:
ATNFSLLKQAGDVEENPGP

SEQ ID NO: 30 - DNA, 2230 bp, synthetic construct
  source 1..2230: mol_type = other DNA; note = AAV VP1 sequence:
atgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 31 - DNA, 561 bp, synthetic construct
  source 1..561: mol_type = other DNA; note = Anti human CD8 DARPIN sequence:
ggaggcggagggtctggcggaggtggaagtggcggaggcggttctggtgggggtggctctggtgggggcggatctgatctgggaaagaagctgctggaagccagcagagccggccaggatgatgaagtgcggatcctgatggccaacggcgccgatgtgaatgcccaggacagatacggcaccacacctctgcatctggccgcctggcatggacacctggaaatcgtggaagtgctgctgaagcacggggccgacgtgaacgccaatgacgtgaagggaaacaccccactgcacctggctgccaatgtgggccatctggaaattgtcgaggtcctgctgaagtatggcgcagacgtcaacgccgccgacaactggggacatactccactgcatctcgcagccttttggggccacctcgagatcgttgaggtgctcctgaaatacggcgctgatgtcaacgctcaggataagttcggcaagacccctttcgacctggccatcgacaacggcaacgaggatattgccgaggtgctgcagaaagctgcaggcggtggcggagct

SEQ ID NO: 32 - DNA, 2791 bp, synthetic construct
  source 1..2791: mol_type = other DNA; note = AAV VP1-CD8 DARPIN fused sequence:
atgatttaaatcaggtatggctgccgatggttatcttcccgattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgggaggcggagggtctggcggaggtggaagtggcggaggcggttctggtgggggtggctctggtgggggcggatctgatctgggaaagaagctgctggaagccagcagagccggccaggatgatgaagtgcggatcctgatggccaacggcgccgatgtgaatgcccaggacagatacggcaccacacctctgcatctggccgcctggcatggacacctggaaatcgtggaagtgctgctgaagcacggggccgacgtgaacgccaatgacgtgaagggaaacaccccactgcacctggctgccaatgtgggccatctggaaattgtcgaggtcctgctgaagtatggcgcagacgtcaacgccgccgacaactggggacatactccactgcatctcgcagccttttggggccacctcgagatcgttgaggtgctcctgaaatacggcgctgatgtcaacgctcaggataagttcggcaagacccctttcgacctggccatcgacaacggcaacgaggatattgccgaggtgctgcagaaagctgcaggcggtggcggagctacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaagccggcaacgcccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 33 - DNA, 1803 bp, synthetic construct
  source 1..1803: mol_type = other DNA; note = AAV VP2/3 sequence:
acggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 34 - DNA, 2230 bp, synthetic construct
  source 1..2230: mol_type = other DNA; note = AAV VP2/3 MET deletion R mutation sequence:
atgatttaaatcaggtaaggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaagccggcaacgcccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 35 - DNA, 7333 bp, synthetic construct
  source 1..7333: mol_type = other DNA; note = The cassette used for the AAV-DARPIN/Vp2-Vp3 MET deletion R mutation construction:
ccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcag

SEQ ID NO: 36 - protein, 737 aa, synthetic construct
  source 1..737: note = VP1/VP2/VP3 WT
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 37 - protein, 924 aa, synthetic construct
  source 1..924: note = VP1 CD8 DARPIN
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTGGGGSGGGGSGGGGSGGGGSGGGGSDLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGHTPLHLAAFWGHLEIVEVLLKYGADVNAQDKFGKTPFDLAIDNGNEDIAEVLQKAAGGGGATNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 38 - protein, 187 aa, synthetic construct
  source 1..187: note = CD8 DARPIN
GGGGSGGGGSGGGGSGGGGSGGGGSDLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGHTPLHLAAFWGHLEIVEVLLKYGADVNAQDKFGKTPFDLAIDNGNEDIAEVLQKAAGGGGA

SEQ ID NO: 39 - DNA, 273 bp, synthetic construct
  source 1..273: mol_type = other DNA; note = TRAC exon 1 The first exon of hTRAC locus
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccag

SEQ ID NO: 40 - protein, 90 aa, synthetic construct
  source 1..90: note = TRAC exon 1 The first exon of hTRAC locus
IQNPDPAVYQLRDSKSSDKSVCLFTDFDSQTNVSQSKDSDVYITDKTVLDMRSMDFKSNSAVAWSNKSDFACANAFNNSIIPEDTFFPSP

SEQ ID NO: 41 - DNA, 76 bp, synthetic construct
  source 1..76: mol_type = other DNA; note = SV40 NLS
atggatccaaaaaagaagagaaaggttgatcccaagaaaaagaggaaagtggatcctaaaaaaaagcggaaagtaa

SEQ ID NO: 42 - protein, 25 aa, synthetic construct
  source 1..25: note = SV40 NLS
MDPKKKRKVDPKKKRKVDPKKKRKV

SEQ ID NO: 43 - DNA, 115 bp, synthetic construct
  source 1..115: mol_type = other DNA; note = Locus area sequence Figure 3B :
cctctccttatttctccctgtctgccaaaaaatctttcccagctcactaagtcagtctcacgcagtcactcattaacccaccaatcactgattgtgccggcacatgaatgcacca

SEQ ID NO: 44 - DNA, 115 bp, synthetic construct
  source 1..115: mol_type = other DNA; note = Locus area complementary sequence Figure 3B
ggagaggaataaagagggacagacggttttttagaaagggtcgagtgattcagtcagagtgcgtcagtgagtaattgggtggttagtgactaacacggccgtgtacttacgtggt

SEQ ID NO: 45 - DNA, 72 bp, synthetic construct
  source 1..72: mol_type = other DNA; note = Sanger sequencing:
cctctccttatttctccctgtctgccaaaaaatctttcccagctcactaagtcagtctcacgcagtcactca

SEQ ID NO: 46 - DNA, 137 bp, synthetic construct
  source 1..137: mol_type = other DNA; note = Locus area sequence Figure 4Ei:
ttttgaagaagatcctattaaataaaagaataagcagtattattaagtagccctgcatttcaggtttccttgagtggcaggccaggcctggccgtgaacgttcactgaaatcatggcctcttggccaagattgatag

SEQ ID NO: 47 - DNA, 137 bp, synthetic construct
  source 1..137: mol_type = other DNA; note = Locus area complementary sequence Figure 4Ei:
aaaacttcttctaggataatttattttcttattcgtcataataattcatcgggacgtaaagtccaaaggaactcaccgtccggtccggaccggcacttgcaagtgactttagtaccggagaaccggttctaactatc

SEQ ID NO: 48 - DNA, 124 bp, synthetic construct
  source 1..124: mol_type = other DNA; note = Sanger sequencing upper panel (single strand) Figure 4Ei:
cctattaaataaaagaataagcagtattattaagtagccctgcatttcaggtttccttgagtggcaggccaggcctggccgtgaacgttcactgaaatcatggcctcttggccaagattgatag

SEQ ID NO: 49 - DNA, 219 bp, synthetic construct
  source 1..219: mol_type = other DNA; note = Locus area sequence Figure 4Eii:
acttcaagagcaacagtgctgtggcctggagcaagcaccgcgcgaaacgcggaagcggagctactaacttcagcctgctgaagcaggctggagacgtggaggagaaccctggacctatggccttaccagtgaccgccttgctcctgccgctggccttgctgctccacgccgccaggccggacatccagatgacacagactacatcctccctgtctgcct

SEQ ID NO: 50 - DNA, 219 bp, synthetic construct
  source 1..219: mol_type = other DNA; note = Locus area complementary sequence Figure 4Eii:
tgaagttctcgttgtcacgacaccggacctcgttcgtggcgcgctttgcgccttcgcctcgatgattgaagtcggacgacttcgtccgacctctgcacctcctcttgggacctggataccggaatggtcactggcggaacgaggacggcgaccggaacgacgaggtgcggcggtccggcctgtaggtctactgtgtctgatgtaggagggacagacgga

SEQ ID NO: 51 - protein, 51 aa, synthetic construct
  source 1..51: note = Predicted residues
NFSLLKQAGDVEENPGPMALPVTALLLPLALLLHAARPDIQMTQTTSSLSA

SEQ ID NO: 52 - DNA, 195 bp, synthetic construct
  source 1..195: mol_type = other DNA; note = Sanger sequencing upper panel (single strand) Figure 4Eii:
acttcaagagcaacagtgctgtggcctggagcaagcaccgcgcgaaacgcggaagcggagctactaacttcagcctgctgaagcaggctggagacgtggaggagaaccctggacctatggccttaccagtgaccgccttgctcctgccgctggccttgctgctccacgccgccaggccggacatccagatgacac